SEQUENCE LISTING (1 sequence)

SEQ ID NO: 1 - protein, 131 aa, Canis lupus
  misc_feature (1)..(131): Canis lupus interleukin-4 precursor
MGLTSQLIPTLVCLLALTSTFVHGHNFNITIKEIIKMLNILTARNDSCMELTVDVFTAPKNTSDKEIFCRAATVLRQIYTHNCSNRYLRGLYRNLSSMANKTCSMNEIKKSTLKDFLERLKVIMQKKYYRH

(19) United States
(12) Patent Application Publication
(10) Pub. No.: US 2024/0270819 A1
(43) Pub. Date: Aug. 15, 2024

(54) IL4/IL13 RECEPTOR MOLECULES FOR VETERINARY USE

(71) Applicant: Elanco US Inc., Greenfield, IN (US)

(72) Inventors: Shyr Jiann LI, Burlingame, CA (US); Lam NGUYEN, Burlingame, CA (US); Qingyi CHU, Burlingame, CA (US); Fawn QIAN, Burlingame, CA (US); Richard CHIN, Burlingame, CA (US); Hangjun ZHAN, Burlingame, CA (US)

(21) Appl. No.: 17/996,549

(22) PCT Filed: Apr. 22, 2021
(86) PCT No.: PCT/US2021/028679

§ 371 Date: Oct. 19, 2022

Related U.S. Application Data
(60) Provisional application No. 63/014,573, filed on Apr. 23, 2020.
(60) Provisional application No. 63/014,090, filed on Apr. 22, 2020.

(51) Int. Cl. C07K 14/715; A61K 38/00; G01N 33/68

(52) U.S. Cl. CPC C07K 14/7155; G01N 33/6869; A61K 38/00; C07K 2319/21; C07K 2319/30; G01N 2333/5406; G01N 2333/5437

(57) ABSTRACT

Provided are various embodiments relating to IL13R/IL4R contiguous polypeptides and IL13R/IL4R heterodimeric proteins from companion animal species and that bind to IL13 and/or IL4, including long-acting contiguous polypeptides and heterodimeric proteins. Such heterodimeric proteins can be used in methods to treat IL13 and/or IL4-induced conditions in companion animals, such as canines, felines, and equines.

### CROSS-REFERENCE TO RELATED APPLICATIONS

This application claims the benefit of U.S. Provisional Application No. 63/014,090, filed Apr. 22, 2020, and U.S. Provisional Application No. 63/014,573, filed Apr. 23, 2020, each of which is incorporated by reference herein in its entirety for any purpose.

### SEQUENCE LISTING

The present application is being filed along with a Sequence Listing in electronic format. The Sequence Listing is provided as a file entitled 2021-04-22_01157-0033-00PCT_ST25.txt created Apr. 22, 2021, which is 614,620 bytes in size. The information in the electronic format of the sequence listing is incorporated herein by reference in its entirety.

### FIELD

This present disclosure relates to contiguous polypeptides and heterodimeric proteins comprising interleukin 4 receptor and interleukin 13 receptor fragments from companion animal species that bind to IL4 and/or IL13 of a companion animal species, for example, canine IL4 and canine IL13, including long-acting molecules with increased serum half-life. The present disclosure also relates to methods of using the contiguous polypeptides and heterodimeric proteins, for example, for treating IL4 and/or IL13-induced conditions or reducing IL4 and/or IL13 signaling activity in cells, for instance in companion animals, such as canines, felines, and equines.

### BACKGROUND

Interleukin 4 (IL4) is a cytokine promoting differentiation of naïve helper T cells to Th2 cells. Interleukin 13 (IL13) has similar effects on immune cells. Both IL4 and IL13 play important roles in T cell-mediated immune responses that are directly associated with allergy, for example, atopic dermatitis and asthma. It is generally understood that IL4 can form a signaling complex either with heterodimeric receptors IL4 receptor subunit alpha (IL4R) and yc or IL4R and IL13 receptor subunit alpha-1 (IL13R). IL13 can form a signaling complex with heterodimeric receptors IL4Ra and IL13Ra1. Extracellular domains of IL4Ra or IL13Ra1 may bind to IL4 and/or IL13 and reduce the free concentrations of the cytokines, thus diminishing the clinical signs and symptoms associated with dermatitis, asthma and other disorders.

Companion species animals, such as cats, dogs, and horses, suffer from many allergic diseases similar to human allergic diseases, including atopic dermatitis and asthma. There remains a need, therefore, for methods and compounds that can be used specifically to bind companion animal IL4 and/or IL13 for treating IL4/IL13-induced conditions and for reducing IL4/IL13 signaling activity.

### SUMMARY

Embodiment 1. A contiguous polypeptide comprising an extracellular domain of an IL13R decoy polypeptide and an extracellular domain of an IL4R polypeptide, wherein the IL13R decoy and/or IL4R polypeptides are from a companion animal species.

Embodiment 2. The contiguous polypeptide of embodiment 1, comprising formula (I) IL13Rd-L1-IL4R-L2-FP, formula (II) IL4R-L1-IL13Rd-L2-FP, formula (III) IL13Rd-L1-FP-L2-IL4R, formula (IV) IL4R-L1-FP-L2-IL13Rd, (V) FP-L1-IL13Rd-L2-IL4R, or formula (VI) FP-L1-IL4R-L2-IL13Rd, wherein:
a) IL13Rd is an extracellular domain of an IL13R decoy polypeptide from the companion animal species,
b) IL4R is an extracellular domain of an IL4R polypeptide from the companion animal species,
c) L1 is a first optional linker,
d) L2 is a second optional linker, and
e) FP is a fusion partner, such as an IgG Fc polypeptide.

Embodiment 3. A contiguous polypeptide comprising an extracellular domain of an IL13R polypeptide and an extracellular domain of an IL4R polypeptide, wherein the IL13R and IL4R polypeptides are from a companion animal species, wherein the contiguous polypeptide comprises the formula (III) IL13R-L1-FP-L2-IL4R, formula (IV) IL4R-L1-FP-L2-IL13R, (V) FP-L1-IL13R-L2-IL4R, or formula (VI) FP-L1-IL4R-L2-IL13R, wherein:
a) IL13R is an extracellular domain of an IL13R polypeptide from the companion animal species,
b) IL4R is an extracellular domain of an IL4R polypeptide from the companion animal species,
c) L1 is a first optional linker,
d) L2 is a second optional linker, and
e) FP is a fusion partner, such as an IgG Fc polypeptide.

Embodiment 4. The contiguous polypeptide of any one of embodiments 1 to 3, wherein the contiguous polypeptide comprises a variant IgG Fc polypeptide from a companion animal species capable of binding to neonatal Fc receptor (FcRn) with an increased affinity relative to the wild-type Fc polypeptide, such as at a low pH.

Embodiment 5. A contiguous polypeptide comprising an extracellular domain of an IL13R polypeptide and an extracellular domain of an IL4R polypeptide, wherein the IL13R and IL4R polypeptides are from a companion animal species, wherein the contiguous polypeptide comprises a variant IgG Fc polypeptide from a companion animal species capable of binding to neonatal Fc receptor (FcRn) with an increased affinity relative to the wild-type Fc polypeptide, such as at a low pH.

Embodiment 6. The contiguous polypeptide of embodiment 5, comprising formula (I) IL13R-L1-IL4R-L2-Fc, formula (II) IL4R-L1-IL13R-L2-Fc, formula (III) IL13R-L1-Fc-L2-IL4R, formula (IV) IL4R-L1-Fc-L2-IL13R, (V) Fc-L1-IL13R-L2-IL4R, or formula (VI) Fc-L1-IL4R-L2-IL13R, wherein:
a) IL13R is an extracellular domain of an IL13R polypeptide from the companion animal species,
b) IL4R is an extracellular domain of an IL4R polypeptide from the companion animal species,
c) L1 is a first optional linker,
d) L2 is a second optional linker, and
e) Fc is a IgG Fc polypeptide.

Embodiment 7. The contiguous polypeptide of any one of the preceding embodiments, wherein the contiguous polypeptide binds to IL13 of the companion animal species with a dissociation constant (Kd) of less than 5×10⁻⁶ M, less than 1×10⁻⁷ M, less than 5×10⁻⁷ M, less than 1×10⁻⁷ M, less than 5×10⁻⁸ M, less than 1×10⁻⁸ M, less than 5×10⁻⁹ M, less than 1×10⁻⁹ M, less than 5×10⁻¹⁰ M, less than 1×10⁻¹⁰ M, less than 5×10⁻¹¹ M, less than 1×10⁻¹¹ M, less than 5×10⁻¹² M, or less than 1×10⁻¹² M, as measured by biolayer interferometry.

Embodiment 8. The contiguous polypeptide of any one of the preceding embodiments, wherein the contiguous polypeptide binds to IL4 of the companion animal species with a dissociation constant (Kd) of less than 5×10⁻⁶ M, less than 1×10⁻⁶ M, less than 5×10⁻⁷ M, less than 1×10⁻⁷ M, less than 5×10⁻⁸ M, less than 1×10⁻⁸ M, less than 5×10⁻⁹ M, less than 1×10⁻⁹ M, less than 5×10⁻¹⁰ M, less than 1×10⁻¹⁰ M, less than 5×10⁻¹¹ M, less than 1×10⁻¹¹ M, less than 5×10⁻¹² M, or less than 1×10⁻¹² M, as measured by biolayer interferometry.

Embodiment 9. The contiguous polypeptide of any one of the preceding embodiments, wherein the contiguous polypeptide reduces IL13 and/or IL4 signaling in the companion animal species.

Embodiment 10. The contiguous polypeptide of any one of the preceding embodiments, wherein the companion animal species is canine, feline, or equine.

Embodiment 11. The contiguous polypeptide of any one of embodiments 3 to 10, wherein the extracellular domain of the IL13R polypeptide is at least 85% identical to the amino acid sequence of SEQ ID NO: 22, SEQ ID NO: 24, SEQ ID NO: 26, SEQ ID NO: 32, SEQ ID NO: 34, or SEQ ID NO: 36.

Embodiment 12. The contiguous polypeptide of any one of embodiments 3 to 11, wherein the extracellular domain of the IL13R polypeptide is at least 90% identical to the amino acid sequence of SEQ ID NO: 22, SEQ ID NO: 24, SEQ ID NO: 26, SEQ ID NO: 32, SEQ ID NO: 34, or SEQ ID NO: 36.

Embodiment 13. The contiguous polypeptide of any one of embodiments 3 to 12, wherein the extracellular domain of the IL13R polypeptide is at least 95% identical to the amino acid sequence of SEQ ID NO: 22, SEQ ID NO: 24, SEQ ID NO: 26, SEQ ID NO: 32, SEQ ID NO: 34, or SEQ ID NO: 36.

Embodiment 14. The contiguous polypeptide of any one of embodiments 3 to 13, wherein the extracellular domain of the IL13R polypeptide is at least 98% identical or at least 99% identical to the amino acid sequence of SEQ ID NO: 22, SEQ ID NO: 24, SEQ ID NO: 26, SEQ ID NO: 32, SEQ ID NO: 34, or SEQ ID NO: 36.

Embodiment 15. The contiguous polypeptide of any one of embodiments 3 to 14, wherein the extracellular domain of the IL13R polypeptide comprises a cysteine at a position corresponding to position 18 of SEQ ID NO: 22, corresponding to position 18 of SEQ ID NO: 24, or corresponding to position 18 of SEQ ID NO: 26.

Embodiment 16. The contiguous polypeptide of any one of embodiments 3 to 15, wherein the extracellular domain of the IL13R polypeptide comprises a cysteine at position 18 of SEQ ID NO: 22, at position 18 of SEQ ID NO: 24, at position 18 of SEQ ID NO: 26, at position 15 of SEQ ID NO: 32, at position 15 of SEQ ID NO: 34, or at position 15 of SEQ ID NO: 36.

Embodiment 17. The contiguous polypeptide of any one of embodiments 3 to 16, wherein the extracellular domain of the IL13R polypeptide comprises an amino acid sequence selected from SEQ ID NO: 32, SEQ ID NO: 34, and SEQ ID NO: 36.

Embodiment 18. The contiguous polypeptide of any one of embodiments 3 to 17, wherein the extracellular domain of the IL13R polypeptide comprises an amino acid sequence selected from SEQ ID NO: 22, SEQ ID NO: 24, and SEQ ID NO: 26.

Embodiment 19. The contiguous polypeptide of any one of embodiments 1, 2, 4, or 7 to 18, wherein the extracellular domain of the IL13R decoy polypeptide is at least 85% identical, at least 90% identical, at least 95% identical, at least 98% identical, or at least 99% identical to the amino acid sequence of SEQ ID NO: 167, SEQ ID NO: 168, or SEQ ID NO: 169.

Embodiment 20. The contiguous polypeptide of any one of the preceding embodiments, wherein the extracellular domain of the IL4R polypeptide is at least 85% identical to the amino acid sequence of SEQ ID NO: 23, SEQ ID NO: 163, SEQ ID NO: 25, SEQ ID NO: 27, SEQ ID NO: 33, SEQ ID NO: 35, or SEQ ID NO: 37.

Embodiment 21. The contiguous polypeptide of any one of the preceding embodiments, wherein the extracellular domain of the IL4R polypeptide is at least 90% identical to the amino acid sequence of SEQ ID NO: 23, SEQ ID NO: 163, SEQ ID NO: 25, SEQ ID NO: 27, SEQ ID NO: 33, SEQ ID NO: 35, or SEQ ID NO: 37.

Embodiment 22. The contiguous polypeptide of any one of the preceding embodiments, wherein the extracellular domain of the IL4R polypeptide is at least 95% identical to the amino acid sequence of SEQ ID NO: 23, SEQ ID NO: 163, SEQ ID NO: 25, SEQ ID NO: 27, SEQ ID NO: 33, SEQ ID NO: 35, or SEQ ID NO: 37.

Embodiment 23. The contiguous polypeptide of any one of the preceding embodiments, wherein the extracellular domain of the IL4R polypeptide is at least 98% identical or at least 99% identical to the amino acid sequence of SEQ ID NO: 23, SEQ ID NO: 163, SEQ ID NO: 25, SEQ ID NO: 27, SEQ ID NO: 33, SEQ ID NO: 35, or SEQ ID NO: 37.

Embodiment 24. The contiguous polypeptide of any one of the preceding embodiments, wherein the extracellular domain of the IL4R polypeptide comprises an amino acid sequence selected from SEQ ID NO: 33, SEQ ID NO: 35, and SEQ ID NO: 37.

Embodiment 25. The contiguous polypeptide of any one of the preceding embodiments, wherein the extracellular domain of the IL4R polypeptide comprises an amino acid sequence selected from SEQ ID NO: 23, SEQ ID NO: 163, SEQ ID NO: 25, and SEQ ID NO: 27.

Embodiment 26. The contiguous polypeptide of any one of embodiments 2 to 4, or 6 to 25, wherein L1 and L2, if present, each independently comprises an amino acid sequence selected from G, GG, GGG, S, SS, SSS, GS, GSGS (SEQ ID NO: 151), GSGSGS (SEQ ID NO: 152), GGS, GGSGGS (SEQ ID NO: 153), GGSGGSGGS (SEQ ID NO: 154), GGGS (SEQ ID NO: 155), GGGSGGGS (SEQ ID NO: 156), GGGSGGGSGGGS (SEQ ID NO: 157), GSS, GSSGSS (SEQ ID NO: 158), GSSGSSGSS (SEQ ID NO: 159), GGSS (SEQ ID NO: 160), GGSSGGSS (SEQ ID NO: 161), and GGSSGGSSGGSS (SEQ ID NO: 162).

Embodiment 27. The contiguous polypeptide of any one of embodiments 2, 3, or 5 to 25, wherein the contiguous polypeptide comprises the sequence selected from SEQ ID NO: 13, SEQ ID NO: 14, SEQ ID NO: 15, SEQ ID NO: 16, SEQ ID NO: 17, SEQ ID NO 18, SEQ ID NO: 19, SEQ ID NO: 20, SEQ ID NO: 21, SEQ ID NO: 28, SEQ ID NO: 29, SEQ ID NO: 30, and SEQ ID NO: 31.

Embodiment 28. A heterodimeric protein comprising:
a) a first contiguous polypeptide comprising at least one IL13R decoy extracellular domain (ECD) and a first Fc polypeptide, and
b) a second contiguous polypeptide comprising at least one IL4R ECD and a second Fc polypeptide, wherein the IL13R decoy ECD and/or the IL4R ECD are from a companion animal species.

Embodiment 29. The heterodimeric protein of embodiment 28, wherein the first Fc polypeptide and/or the second Fc polypeptide is a variant IgG Fc polypeptide from a companion animal species capable of binding to neonatal Fc receptor (FcRn) with an increased affinity relative to the wild-type Fc polypeptide, such as at a low pH.

Embodiment 30. A heterodimeric protein comprising:
a) a first contiguous polypeptide comprising at least one IL13R extracellular domain (ECD) and a first Fc polypeptide, and
b) a second contiguous polypeptide comprising at least one IL4R ECD and a second Fc polypeptide wherein the IL13R ECD and/or the IL4R ECD are from a companion animal species, and wherein the first Fc polypeptide and/or the second Fc polypeptide is a variant IgG Fc polypeptide from a companion animal species capable of binding to neonatal Fc receptor (FcRn) with an increased affinity relative to the wild-type Fc polypeptide, such as at a low pH.

Embodiment 31. The heterodimeric protein of any one of embodiments 28 to 30, wherein the first contiguous polypeptide and/or the second contiguous polypeptide comprises one, two, three, or four IL4R ECDs and/or one, two, three, or four IL13R ECDs or IL13R decoy ECDs.

Embodiment 32. The heterodimeric protein of any one of embodiments 28 to 31, wherein the first contiguous polypeptide and/or the second contiguous polypeptide further comprises at least one binding partner other than IL4R ECD, IL13R ECD, or IL13R decoy ECD.

Embodiment 33. The heterodimeric protein of embodiment 32, wherein the at least one binding partner comprises IL5, IL6, IL17, IL22, IL31, LFA-1, TNF-α, TSLP, and/or IgE.

Embodiment 34. The heterodimeric protein of any one embodiments 28 to 33, wherein the heterodimeric protein binds to IL13 and/or IL4 with a dissociation constant (Kd) of less than 5×10⁻⁶ M, less than 1×10⁻⁶ M, less than 5×10⁻⁷ M, less than 1×10⁻⁷ M, less than 5×10⁻⁸ M, less than 1×10⁻⁸ M, less than 5×10⁻⁹ M, less than 1×10⁻⁹ M, less than 5×10⁻¹⁰ M, less than 1×10⁻¹⁰ M, less than 5×10₋₁₁ M, less than 1×10⁻¹¹ M, less than 5×10⁻¹² M, or less than 1×10⁻¹² M, as measured by biolayer interferometry.

Embodiment 35. The heterodimeric protein of any one of embodiments 28 to 34, wherein the heterodimeric protein reduces IL13 and/or IL4 signaling in a companion animal species.

Embodiment 36. The heterodimeric protein of any one embodiments 28 to 35, wherein the companion animal species is canine, feline, or equine.

Embodiment 37. The heterodimeric protein of any one of embodiments 30 to 36, wherein the amino acid sequence of the at least one IL13R ECD is at least 85% identical, at least 90% identical, at least 95% identical, or at least 98% identical to the amino acid sequence of SEQ ID NO: 22, SEQ ID NO: 24, SEQ ID NO: 26, SEQ ID NO: 32, SEQ ID NO: 34, or SEQ ID NO: 36.

Embodiment 38. The heterodimeric protein of any one of the embodiments 30 to 37, wherein the amino acid sequence of the at least one IL13R ECD comprises a cysteine at a position corresponding to position 18 of SEQ ID NO: 22, corresponding to position 18 of SEQ ID NO: 24, or corresponding to position 18 of SEQ ID NO: 26.

Embodiment 39. The heterodimeric protein of any one of embodiments 30 to 38, wherein the amino acid sequence of the at least one IL13R ECD comprises a cysteine at position 18 of SEQ ID NO: 22, at position 18 of SEQ ID NO: 24, at position 18 of SEQ ID NO: 26, at position 15 of SEQ ID NO: 32, at position 15 of SEQ ID NO: 34, or at position 15 of SEQ ID NO: 36.

Embodiment 40. The heterodimeric protein of any one of embodiments 30 to 39, wherein the at least one IL13R ECD comprises an amino acid sequence selected from SEQ ID NO: 22, SEQ ID NO: 24, SEQ ID NO: 26, SEQ ID NO: 32, SEQ ID NO: 34, and SEQ ID NO: 36.

Embodiment 41. The heterodimeric protein of any one of embodiments 28, 29, or 31 to 36, wherein the extracellular domain of the IL13R decoy polypeptide is at least 85% identical, at least 90% identical, at least 95% identical, at least 98% identical, or at least 99% identical to the amino acid sequence of SEQ ID NO: 167, SEQ ID NO: 168, or SEQ ID NO: 169.

Embodiment 42. The heterodimeric protein of any one of embodiments 28 to 41, wherein the amino acid sequence of the at least one IL4R ECD is at least 85% identical, at least 90% identical, at least 95% identical, or at least 98% identical to the amino acid sequence of SEQ ID NO: 23, SEQ ID NO: 163, SEQ ID NO: 25, SEQ ID NO: 27, SEQ ID NO: 33, SEQ ID NO: 35, or SEQ ID NO: 37.

Embodiment 43. The heterodimeric protein of any one of embodiments 28 to 42, wherein the at least one IL4R ECD comprises an amino acid sequence selected from SEQ ID NO: 23, SEQ ID NO: 163, SEQ ID NO: 25, SEQ ID NO: 27, SEQ ID NO: 33, SEQ ID NO: 35, and SEQ ID NO: 37.

Embodiment 44. The heterodimeric protein of any one of embodiments 28 to 43, wherein the first Fc polypeptide or the second Fc polypeptide comprises a knob mutation.

Embodiment 45. The heterodimeric protein of any one of embodiments 28 to 44, wherein the first Fc polypeptide or the second Fc polypeptide comprises a hole mutation.

Embodiment 46. The heterodimeric protein of any one of embodiments 28 to 45, wherein the first Fc polypeptide or the second Fc polypeptide comprises:
a) an amino acid substitution at a position corresponding to position 138 of SEQ ID NO: 38, position 137 of SEQ ID NO: 39, position 137 of SEQ ID NO: 40, or position 138 of SEQ ID NO: 41; and/or
b) an amino acid substitution at a position corresponding to position 154 of SEQ ID NO: 42, SEQ ID NO: 43, SEQ ID NO: 44, SEQ ID NO: 45, or SEQ ID NO: 46; and/or
c) an amino acid substitution at a position corresponding to position 130 of SEQ ID NO: 47, SEQ ID NO: 48, SEQ ID NO: 49, SEQ ID NO: 50, SEQ ID NO: 51, SEQ ID NO: 52, or SEQ ID NO: 53.

Embodiment 47. The heterodimeric protein of any one of embodiments 28 to 46, wherein the first Fc polypeptide or the second Fc polypeptide comprises:
a) a tryptophan at a position corresponding to position 138 of SEQ ID NO: 38, position 137 of SEQ ID NO: 39, position 137 of SEQ ID NO: 40, or position 138 of SEQ ID NO: 41; and/or
b) a tryptophan at a position corresponding to position 154 of SEQ ID NO: 42, SEQ ID NO: 43, SEQ ID NO: 44, SEQ ID NO: 45, or SEQ ID NO: 46; and/or
c) a tryptophan at a position corresponding to position 130 of SEQ ID NO: 47, SEQ ID NO: 48, SEQ ID NO: 49, SEQ ID NO: 50, SEQ ID NO: 51, SEQ ID NO: 52, or SEQ ID NO: 53.

Embodiment 48. The heterodimeric protein of any one of embodiments 28 to 47, wherein the first Fc polypeptide or the second Fc polypeptide comprises:
a) an amino acid substitution at position 138 of SEQ ID NO: 38, position 137 of SEQ ID NO: 39, position 137 of SEQ ID NO: 40, or position 138 of SEQ ID NO: 41; and/or
b) an amino acid substitution at position 154 of SEQ ID NO: 42, SEQ ID NO: 43, SEQ ID NO: 44, SEQ ID NO: 45, or SEQ ID NO: 46; and/or
c) an amino acid substitution at position 130 of SEQ ID NO: 47, SEQ ID NO: 48, SEQ ID NO: 49, SEQ ID NO: 50, SEQ ID NO: 51, SEQ ID NO: 52, or SEQ ID NO: 53.

Embodiment 49. The heterodimeric protein of any one embodiments 28 to 48, wherein the first Fc polypeptide or the second Fc polypeptide comprises:
a) a tryptophan at position 138 of SEQ ID NO: 38, position 137 of SEQ ID NO: 39, position 137 of SEQ ID NO: 40, or position 138 of SEQ ID NO: 41; and/or
b) a tryptophan at position 154 of SEQ ID NO: 42, SEQ ID NO: 43, SEQ ID NO: 44, SEQ ID NO: 45, or SEQ ID NO: 46; and/or
c) a tryptophan at position 130 of SEQ ID NO: 47, SEQ ID NO: 48, SEQ ID NO: 49, SEQ ID NO: 50, SEQ ID NO: 51, SEQ ID NO: 52, or SEQ ID NO: 53.

Embodiment 50. The heterodimeric protein of any one of embodiments 28 to 49, wherein the first Fc polypeptide or the second Fc polypeptide comprises:
a) an amino acid substitution at a position corresponding to position 138 and/or position 140 and/or position 181 of SEQ ID NO: 38, position 137 and/or position 139 and/or position 180 of SEQ ID NO: 39, position 137 and/or position 139 and/or position 180 of SEQ ID NO: 40, or position 138 and/or position 140 and/or position 181 of SEQ ID NO: 41; and/or
b) an amino acid substitution at a position corresponding to position 154 and/or position 156 and/or position 197 of SEQ ID NO: 42, SEQ ID NO: 43, SEQ ID NO: 44, SEQ ID NO: 45, or SEQ ID NO: 46; and/or
c) an amino acid substitution at a position corresponding to position 130 and/or position 132 and/or position 173 of SEQ ID NO: 47, SEQ ID NO: 48, SEQ ID NO: 49, SEQ ID NO: 50, SEQ ID NO: 51, SEQ ID NO: 52, or SEQ ID NO: 53.

Embodiment 51. The heterodimeric protein of any one of embodiments 28 to 50, wherein the first Fc polypeptide or the second Fc polypeptide comprises:
a) a serine at a position corresponding to position 138 and/or an alanine at a position corresponding to position 140 and/or a threonine at a position corresponding to position 181 of SEQ ID NO: 38, a serine at a position corresponding to position 137 and/or an alanine at a position corresponding to position 139 and/or a threonine at a position corresponding to position 180 of SEQ ID NO: 39, a serine at a position corresponding to position 137 and/or an alanine at a position corresponding to position 139 and/or a threonine at a position corresponding to position 180 of SEQ ID NO: 40, or a serine at a position corresponding to position 138 and/or an alanine at a position corresponding to position 140 and/or a threonine at a position corresponding to position 181 of SEQ ID NO: 41; and/or
b) a serine at a position corresponding to position 154 and/or an alanine at a position corresponding to position 156 and/or a threonine at a position corresponding to position 197 of SEQ ID NO: 42, SEQ ID NO: 43, SEQ ID NO: 44, SEQ ID NO: 45, or SEQ ID NO: 46; and/or
c) a serine at a position corresponding to position 130 and/or an alanine at a position corresponding to position 132 and/or a threonine at a position corresponding to position 173 of SEQ ID NO: 47, SEQ ID NO: 48, SEQ ID NO: 49, SEQ ID NO: 50, SEQ ID NO: 51, SEQ ID NO: 52, or SEQ ID NO: 53.

Embodiment 52. The heterodimeric protein of any one of embodiments 28 to 51, wherein the first Fc polypeptide or the second Fc polypeptide comprises:
a) an amino acid substitution at position 138 and/or position 140 and/or position 181 of SEQ ID NO: 38, position 137 and/or position 139 and/or position 180 of SEQ ID NO: 39, position 137 and/or position 139 and/or position 180 of SEQ ID NO: 40, or position 138 and/or position 140 and/or position 181 of SEQ ID NO: 41; and/or
b) an amino acid substitution at position 154 and/or position 156 and/or position 197 of SEQ ID NO: 42, SEQ ID NO: 43, SEQ ID NO: 44, SEQ ID NO: 45, or SEQ ID NO: 46; and/or
c) an amino acid substitution at position 130 and/or position 132 and/or position 173 of SEQ ID NO: 47, SEQ ID NO: 48, SEQ ID NO: 49, SEQ ID NO: 50, SEQ ID NO: 51, SEQ ID NO: 52, or SEQ ID NO: 53.

Embodiment 53. The heterodimeric protein of any one of embodiments 28 to 52, wherein the first Fc polypeptide or the second Fc polypeptide comprises:
a) a serine at position 138 and/or an alanine at position 140 and/or a threonine at position 181 of SEQ ID NO: 38, a serine at position 137 and/or an alanine at position 139 and/or a threonine at position 180 of SEQ ID NO: 39, a serine at position 137 and/or an alanine at position 139 and/or a threonine at position 180 of SEQ ID NO: 40, or a serine at position 138 and/or an alanine at position 140 and/or a threonine at position 181 of SEQ ID NO: 41; and/or
b) a serine at position 154 and/or an alanine at position 156 and/or a threonine at position 197 of SEQ ID NO: 42, SEQ ID NO: 43, SEQ ID NO: 44, SEQ ID NO: 45, or SEQ ID NO: 46; and/or
c) a serine at position 130 and/or an alanine at position 132 and/or a threonine at position 173 of SEQ ID NO: 47, SEQ ID NO: 48, SEQ ID NO: 49, SEQ ID NO: 50, SEQ ID NO: 51, SEQ ID NO: 52, or SEQ ID NO: 53.

Embodiment 54. The heterodimeric protein of any one of embodiments 28 to 53, wherein the first Fc polypeptide or the second Fc polypeptide comprises the amino acid sequence of SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, or SEQ ID NO: 101.

Embodiment 55. The heterodimeric protein of any one of embodiments 28 to 54, wherein the first contiguous polypeptide comprises the amino acid sequence of SEQ ID NO: 103, SEQ ID NO: 105, SEQ ID NO: 107, SEQ ID NO: 109, SEQ ID NO: 111, or SEQ ID NO: 113.

Embodiment 56. The heterodimeric protein of any one of embodiments 28 to 55, wherein the second contiguous polypeptide comprises the amino acid sequence of SEQ ID NO: 102, SEQ ID NO: 104, SEQ ID NO: 106, SEQ ID NO: 108, SEQ ID NO: 110, or SEQ ID NO: 112.

Embodiment 57. The contiguous polypeptide or heterodimeric protein of any one of the preceding embodiments, wherein the variant IgG Fc polypeptide binds to FcRn with an affinity greater than the wild-type IgG Fc polypeptide, as measured by biolayer interferometry, surface plasmon resonance, or any protein-protein interaction tool at a pH in the range of from about 5.0 to about 6.5, such as at a pH of about 5.0, a pH of about 5.2, a pH of about 5.5, a pH of about 6.0, a pH of about 6.2, or a pH of about 6.5.

Embodiment 58. The contiguous polypeptide or heterodimeric protein of any one of the preceding embodiments, wherein the variant IgG Fc polypeptide binds to FcRn with a dissociation constant (Kd) of less than 5×10⁻⁶ M, less than 1×10⁻⁶ M, less than 5×10⁻⁷ M, less than 1×10⁻⁷ M, less than 5×10⁻⁸ M, less than 1×10⁻⁸ M, less than 5×10⁻⁹ M, less than 1×10⁻⁹ M, less than 5×10⁻¹⁰ M, less than 1×10⁻¹⁰ M, less than 5×10⁻¹¹ M, less than 1×10⁻¹¹ M, less than 5×10⁻¹² M, or less than 1×10⁻¹² M, as measured by biolayer interferometry, surface plasmon resonance, or any protein-protein interaction tool at a pH in the range of from about 5.0 to about 6.5, such as at a pH of about 5.0, a pH of about 5.5, a pH of about 6.0, or a pH of about 6.5.

Embodiment 59. The contiguous polypeptide or heterodimeric protein of any one of the preceding embodiments, wherein the contiguous polypeptide or heterodimeric protein has increased serum half-life relative to a contiguous polypeptide or heterodimeric protein comprising a wild-type Fc polypeptide.

Embodiment 60. The contiguous polypeptide or heterodimeric protein of any one of the preceding embodiments, wherein the variant IgG Fc polypeptide binds to FcRn with an increased affinity relative to the wild-type Fc polypeptide, and wherein the contiguous polypeptide or heterodimeric protein has increased serum half-life relative to a contiguous polypeptide or heterodimeric protein comprising a wild-type Fc polypeptide.

Embodiment 61. The contiguous polypeptide or heterodimeric protein of any one of the preceding embodiments, wherein the variant IgG Fc polypeptide comprises:
a) a tyrosine or a phenylalanine at a position corresponding to position 23 of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53;
b) a tyrosine at a position corresponding to position 82 of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53;
c) a tyrosine at a position corresponding to position 82 and a histidine at a position corresponding to position 207 of SEQ ID NO: 39, SEQ ID NO: 40, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53;
d) a tyrosine at a position corresponding to position 82 and a tyrosine at a position corresponding to position 207 of SEQ ID NO: 39, SEQ ID NO: 40, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53;
e) a tyrosine at a position corresponding to position 207 of SEQ ID NO: 39, SEQ ID NO: 40, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53;
f) a tyrosine at a position corresponding to position 82 and a histidine at a position corresponding to position 208 of SEQ ID NO: 38 or SEQ ID NO: 41;
g) a tyrosine at a position corresponding to position 82 and a tyrosine at a position corresponding to position 208 of SEQ ID NO: 38 or SEQ ID NO: 41; or
h) a tyrosine at a position corresponding to position 208 of SEQ ID NO: or SEQ ID NO: 41.

Embodiment 62. The contiguous polypeptide or heterodimeric protein of any one of the preceding embodiments, wherein the variant IgG Fc polypeptide comprises:
a) a tyrosine or a phenylalanine at position 23 of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53;
b) a tyrosine at position 82 of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53;
c) a tyrosine at position 82 and a histidine at position 207 of SEQ ID NO: 39, SEQ ID NO: 40, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53;
d) a tyrosine at position 82 and a tyrosine at position 207 SEQ ID NO: 39, SEQ ID NO: 40, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53;
e) a tyrosine at position 207 of SEQ ID NO: 39, SEQ ID NO: 40, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53;
f) a tyrosine at position 82 and a histidine at position 208 of SEQ ID NO: 38 or SEQ ID NO: 41;
g) a tyrosine at position 82 and a tyrosine at position 208 of SEQ ID NO: 38 or SEQ ID NO: 41; or
h) a tyrosine at position 208 of SEQ ID NO: 38 or SEQ ID NO: 41.

Embodiment 63. The contiguous polypeptide or heterodimeric protein of any one of the preceding embodiments, wherein the variant IgG Fc polypeptide comprises an amino acid sequence of SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, or SEQ ID NO: 129.

Embodiment 64. The contiguous polypeptide or heterodimeric protein of any one of the preceding embodiments comprising the amino acid sequence of SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, or SEQ ID NO: 183.

Embodiment 65. An isolated polypeptide comprising the amino acid sequence of SEQ ID NO: SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, or SEQ ID NO: 183.

Embodiment 66. The contiguous polypeptide, heterodimeric protein, or polypeptide of any one of the preceding embodiments wherein the contiguous polypeptide, heterodimeric protein, or polypeptide is sialyated.

Embodiment 67. An isolated nucleic acid encoding the contiguous polypeptide, heterodimeric protein, or polypeptide of any one of the preceding embodiments.

Embodiment 68. A host cell comprising the nucleic acid of embodiment 67.

Embodiment 69. A host cell expressing the contiguous polypeptide, heterodimeric protein, or polypeptide of any one of embodiments 1 to 66.

Embodiment 70. A method comprising culturing the host cell of embodiment 68 or 69 and isolating the polypeptide, contiguous polypeptide, the first contiguous polypeptide, the second contiguous polypeptide, or the first contiguous polypeptide and the second contiguous polypeptide.

Embodiment 71. A pharmaceutical composition comprising the contiguous polypeptide, heterodimeric protein, or polypeptide of any one of embodiments 1 to 66 and a pharmaceutically acceptable carrier.

Embodiment 72. A method of treating a companion animal species having an IL13 and/or IL4-induced condition, the method comprising administering to the companion animal species a therapeutically effective amount of the contiguous polypeptide, heterodimeric protein, or polypeptide of any one of embodiments 1 to 66 or the pharmaceutical composition of embodiment 71.

Embodiment 73. The method of embodiment 72, wherein the companion animal species is canine, feline, or equine.

Embodiment 74. The method of embodiment 72 or 73, wherein the IL13 and/or IL4-induced condition is a pruritic or allergic condition, such as atopic dermatitis, pruritus, asthma, psoriasis, scleroderma, or eczema.

Embodiment 75. The method of any one of embodiments 72 to 74, wherein the contiguous polypeptide, the heterodimeric protein, the polypeptide, or the pharmaceutical composition is administered parenterally.

Embodiment 76. The method of any one of embodiments 72 to 75, wherein the heterodimeric protein or the pharmaceutical composition is administered by an intramuscular route, an intraperitoneal route, an intracerebrospinal route, a subcutaneous route, an intra-arterial route, an intrasynovial route, an intrathecal route, or an inhalation route.

Embodiment 77. The method of any one of embodiments 72 to 76, wherein the method further comprises administering a Jak inhibitor, a PI3K inhibitor, an AKT inhibitor, or a MAPK inhibitor.

Embodiment 78. The method of any one of embodiments 62 to 77, wherein the method further comprises administering one or more antibodies selected from an anti-IL17 antibody, an anti-IL31 antibody, an anti-TNFα antibody, an anti-CD20 antibody, an anti-CD19 antibody, an anti-CD25 antibody, an anti-IL4 antibody, an anti-IL13 antibody, an anti-IL23 antibody, an anti-IgE antibody, an anti-CD11α antibody, anti-IL6R antibody, anti-α4-Intergrin antibody, an anti-IL12 antibody, an anti-IL10 antibody, an anti-IL5 antibody, an anti-IL5R antibody, an anti-IL22 antibody, an anti-IL22R antibody, an anti-IL33 antibody, an anti-IL33R antibody, an anti-TSLP antibody, an anti-TSLPR antibody, and an anti-BlyS antibody.

Embodiment 79. A method of reducing IL13 and/or IL4 signaling activity in a cell, the method comprising exposing the cell to the contiguous polypeptide, heterodimeric protein, or polypeptide of any one of embodiments 1 to 66 or the pharmaceutical composition of embodiment 71 under conditions permissive for binding of the heterodimeric protein to IL13 and/or IL4, thereby (a) reducing binding of IL/4 and/or IL-13 to native IL13 receptor and/or native IL-4 receptor and reducing IL13- and/or IL-4-mediated signaling.

Embodiment 80. The method of embodiment 79, wherein the cell is exposed to the heterodimeric protein or the pharmaceutical composition ex vivo.

Embodiment 81. The method of embodiment 79, wherein the cell is exposed to the heterodimeric protein or the pharmaceutical composition in vivo.

Embodiment 82. The method of any one of embodiments 79 to 81, wherein the cell is a canine cell, a feline cell, or an equine cell.

Embodiment 83. A method for detecting IL13 or IL4 in a sample from a companion animal species comprising contacting the sample with the contiguous polypeptide, heterodimeric protein, or polypeptide of any one of embodiments 1 to 66 or the pharmaceutical composition of embodiment 71 under conditions permissive for binding of the heterodimeric protein to IL13 and/or IL4, and detecting whether a complex is formed between the heterodimeric protein and IL13 and/or IL4 in the sample.

Embodiment 84. The method of embodiment 83, wherein the sample is a biological sample obtained from a canine, a feline, or an equine.

### BRIEF DESCRIPTION OF THE DRAWINGS

FIG. 1 is a graph of canine IL4RECD-IL13RECD-Fc sequential binding to canine IL4 and IL13 or canine IL13 and IL4 using concentrations of 30 μg/mL of IL4 and IL13 in PBS.

FIG. 2 is a graph of canine IL13RECD-IL4RECD-Fc sequential binding to canine IL4 and IL13 or canine IL13 and IL4 using concentrations of 30 μg/mL of IL4 and IL13 in PBS.

FIG. 3 is a graph of canine IL4RECD-IL13RECD-Fc neutralizing canine IL4 activity in a TF1 cell proliferation assay. Canine IL4 (50 ng/mL or 3.85 nM) was used in the assay.

FIG. 4 shows a Biacore sensorgram of various concentrations of canine FcRn (12.5, 25, 50, 100, and 200 nM) binding to wild-type canine IgG-B Fc polypeptide.

FIG. 5 shows a Biacore sensorgram of various concentrations of canine FcRn (12.5, 25, 50, 100, and 200 nM) binding to variant canine IgG-B Fc polypeptide L(23)Y.

FIG. 6 shows a Biacore sensorgram of various concentrations of canine FcRn (12.5, 25, 50, 100, and 200 nM) binding to variant canine IgG-B Fc polypeptide L(23)F.

FIG. 7 shows a Biacore sensorgram of various concentrations of canine FcRn (12.5, 25, 50, 100, and 200 nM) binding to variant canine IgG-B Fc polypeptide L(23)M.

FIG. 8 shows a Biacore sensorgram of various concentrations of canine FcRn (12.5, 25, 50, 100, and 200 nM) binding to variant canine IgG-B Fc polypeptide YTE.

FIG. 9 is an OctetRed sensorgram of chimeric variant canine IgG-A Fc F00 antibody (A) and IgG-D Fc F00 antibody (B) binding to canine FcRn compared to that of chimeric variant canine IgG-A Fc without the Phe mutation (C) and IgG-D Fc without the Phe mutation (D).

FIG. 10 shows the serum pharmacokinetics profiles for chimeric variant canine IgG-A F00 antibody (“IgG-A F00”; n=2) and chimeric variant canine IgG-A without the Phe mutation (“IgG-A”; n=2) after subcutaneous administration to rats at 2 mg/kg.

FIG. 11 is an OctetRed sensorgram of chimeric antibodies with variant canine IgG-B Fcs (0Y0, 0YH, 0YY, or 00Y) binding to canine FcRn compared to that of chimeric antibody with a wild-type canine IgG-B.

FIG. 12 is a chart showing percent antibody normalized over time resulting from the in vivo pharmacokinetic study in dog as described in Example 13.

### DESCRIPTION OF CERTAIN SEQUENCES

Table 1 provides a listing of certain sequences referenced herein.

TABLE 1
Description of Certain Sequences

| SEQ ID | | |
|---|---|---|
| NO: | SEQUENCE | DESCRIPTION |
| 1 | MGLTSQLIPTLVCLLALTSTFVHGHNENITIKEII | Canis lupus |
| | KMLNILTARNDSCMELTVDVFTAPKNTSDKEIFCR | interleukin-4 precursor |
| | AATVLRQIYTHNCSNRYLRGLYRNLSSMANKTCSM | |
| | NEIKKSTLKDFLERLKVIMQKKYYRH | |
| 2 | MDLTSQLIPALVCLLAFTSTFVHGQNENNTLKEII | Felis catus |
| | KTLNILTARNDSCMELTMDVLAAPKNTSDKEIFCR | interleukin-4 precursor |
| | ATTVLRQIYTHHNCSTKFLKGLDRNLSSMANRTCS | |
| | VNEVKKCTLKDFLERLKAIMQKKYSKH | |
| 3 | MGLTYQLIPALVCLLACTSNFIQGCKYDITLQEII | Equus caballus interleukin-4 |
| | KTLNNLTDGKGKNSCMELTVADAFAGPKNTDGKEI | precursor |
| | CRAAKVLQQLYKRHDRSLIKECLSGLDRNLKGMAN | |
| | GTCCTVNEAKKSTLKDFLERLKTIMKEKYSKC | |
| 4 | MALWLTVVIALTCLGGLASPSPVTPSPTLKELIEE | Canis lupus |
| | LVNITQNQASLCNGSMVWSVNLTAGMYCAALESLI | interleukin-13 precursor |
| | NVSDCSAIQRTQRMLKALCSQKPAAGQISSERSRD | |
| | TKIEVIQLVKNLLTYVRGVYRHGNER | |
| 5 | MWFLDSTRQSGDQGGRRHTWPIKATARGQGHKPLS | Felis catus |
| | LGQPTCPLLAPPVLALGSMALWLTVVIALTCLGGL | interleukin-13 precursor |
| | ASPGPHSRRELKELIEELVNITQNQVSLCNGSMVW | |
| | SVNLTTGMYCAALESLINVSDCTAIQRTQRMLKAL | |
| | CTQKPSAGQTASERSRDTKIEVIQLVKNLLNHLRR | |
| | NFRHGNFK | |
| 6 | MALWLTAVIALACLGGLASPAPLPSSMALKELIKE | Equus caballus interleukin- |
| | LVNITQNQAPLCNGSMVWSVNLTADTYCRALESLS | 13 precursor |
| | NVSTCSAIQNTRKMLTKLCPHQLSAGQVSSERARD | |
| | TKIEVIVLVKDLLKNLRKIFHGGKHVDA | |
| 7 | MGRLCSGLTFPVSCLVLVWVASSGSVKVLHEPSCF | Canis lupus |
| | SDYISTSVCQWKMDHPTNCSAELRLSYQLDEMGSE | interleukin-4 receptor |
| | NHTCVPENREDSVCVCSMPIDDAVEADVYQLDLWA | subunit alpha |
| | GQQLLWSGSFQPSKHVKPRTPGNLTVHPNISHTWL | |
| | LMWTNPYPTENHLHSELTYMVNVSNDNDPEDFKVY | |
| | NVTYMGPTLRLAASTLKSGASYSARVRAWAQTYNS | |
| | TWSDWSPSTTWLNYYEPWEQHLPLGVSISCLVILA | |
| | ICLSCYFSIIKIKKGWWDQIPNPAHSPLVAIVIQD | |
| | SQVSLWGKRSRGQEPAKCPHWKTCLTKLLPCLLEH | |
| | GLGREEESPKTAKNGPLQGPGKPAWCPVEVSKTIL | |
| | WPESISVVQCVELSEAPVDNEEEEEVEEDKRSLCP | |
| | SLEGSGGSFQEGREGIVARLTESLFLDLLGGENGG | |
| | FCPQGLEESCLPPPSGSVGAQMPWAQFPRAGPRAA | |
| | PEGPEQPRRPESALQASPTQSAGSSAFPEPPPVVT | |
| | DNPAYRSFGSFLGQSSDPGDGDSDPELADRPGEAD | |
| | PGIPSAPQPPEPPAALQPEPESWEQILRQSVLQHR | |
| | AAPAPGPGPGSGYREFTCAVKQGSAPDAGGPGFGP | |
| | SGEAGYKAFCSLLPGGATCPGTSGGEAGSGEGGYK | |
| | PFQSLTPGCPGAPTPVPVPLFTFGLDTEPPGSPQD | |
| | SLGAGSSPEHLGVEPAGKEEDSRKTLLAPEQATDP | |
| | LRDDLASSIVYSALTCHLCGHLKQWHDQEERGKAH | |
| | IVPSPCCGCCCGDRSSLLLSPLRAPNVLPGGVLLE | |
| | ASLSPASLVPSGVSKEGKSSPFSQPASSSAQSSSQ | |
| | TPKKLAVLSTEPTCMSAS | |
| 8 | MGRLCSGLTFPVSCLILMWAAGSGSVKVLRAPTCF | Felis catus |
| | SDYFSTSVCQWNMDAPTNCSAELRLSYQLNEMGSE | interleukin-4 receptor |
| | NRTCVPENGEGAACACSMLMDDFVEADVYQLHLWA | subunit alpha |
| | GTQLLWSGSFKPSSHVKPRAPGNLTVHPNVSHTWL | |
| | LRWSNPYPPENHLHAELTYMVNISSEDDPTDVSVC | |
| | ASGFLCHLLGLRRVETGAPGARLPPWLCAPRPRRV | |
| | PGSQCAVISCCRWVLIALTSRGGRWRLTPGLRSQT | |
| | RYVSVAEGLFGATPRVLCPGTQAGLASAAREQMSP | |
| | DPSAFHSIDYEPWEQHLPLGVSISCLVILAVCLSC | |
| | YLSVIKIKKEWWDQIPNPAHSHLVAIVIQDPQVSL | |
| | WGKRSRGQEPAKCPHWKTCLRKLLPCLLEHGMERK | |
| | EDPSKIARNGPSQCSGKSAWCPVEVSKTILWPESI | |
| | SVVRCVELLEAPVESEEEEEEEEDKGSFCPSPVNL | |
| | EDSFQEGREGIAARLTESLFMDLLGVEKGGFGPQG | |
| | SLESWFPPPSGSAGAQMPWAEFPGPGPQEASPQGK | |
| | EQPFDPRSDPLATLPQSPASPTFPETPPVVTDNPA | |
| | YRSFGTFQGRSSGPGECDSGPELAGRLGEADPGIP | |
| | AAPQPSEPPSALQPEAETWEQILRQRVLQHRGAPA | |
| | PAPGSGYREFVCAVRQGSTQDSGVGDFGPSEEAGY | |
| | KAFSSLLTSGAVCPESGGEAGSGDGGYKPFQSLTP | |
| | GCPGAPAPVPVPLFTFGLDAEPPHCPQDSPLPGSS | |
| | PEPAGKAQDSHKTPPAPEQAADPLRDDLASGIVYS | |
| | ALTCHLCGHLKQCHGQEEGGEAHPVASPCCGCCCG | |
| | DRSSPLVSPLRAPDPLPGGVPLEASLSPASPAPLA | |
| | VSEEGPPSLCFQPALSHAHSSSQTPKKVAMLSPEP | |
| | TCTMAS | |
| 9 | MGCLCPGLTLPVSCLILVWAAGSGSVKVLHLTACF | Equus caballus |
| | SDYISASTCEWKMDRPTNCSAQLRLSYQLNDEFSD | interleukin-4 receptor |
| | NLTCIPENREDEVCVCRMLMDNIVSEDVYELDLWA | subunit alpha |
| | GNQLLWNSSFKPSRHVKPRAPQNLTVHAISHTWLL | |
| | TWSNPYPLKNHLWSELTYLVNISKEDDPTDFKIYN | |
| | VTYMDPTLRVTASTLKSRATYSARVKARAQNYNST | |
| | WSEWSPSTTWHNYYEQPLEQRLPLGVSISCVVILA | |
| | ICLSCYFSIIKIKKEWWDQIPNPAHSPLVAIVLQD | |
| | SQVSLWGKQSRGQEPAKCPRWKTCLTKLLPCLLEH | |
| | GLQKEEDSSKTVRNGPFQSPGKSAWHTVEVNHTIL | |
| | RPEIISVVPCVELCEAQVESEEEEVEEDRGSFCPS | |
| | PESSGSGFQEGREGVAARLTESLFLGLLGAENGAL | |
| | GESCLLPPLGSAHMPWARISSAGPQEAASQGEEQP | |
| | LNPESNPLATLTQSPGSLAFTEAPAVVADNPAYRS | |
| | FSNSLSQPRGPGELDSDPQLAEHLGQVDPSIPSAP | |
| | QPSEPPTALQPEPETWEQMLRQSVLQQGAAPAPAS | |
| | APTGGYREFAQAVKQGGGAAGSGPSGEAGYKAFSS | |
| | LLAGSAVCPGQSGVEASSGEGGYRPYESPDPGAPA | |
| | PVPVPLFTFGLDVEPPHSPQNSLLPGGSPELPGPE | |
| | PTVKGEDPRKPLLSAQQATDSLRDDLGSGIVYSAL | |
| | TCHLCGHLKQCHGQEEHGEAHTVASPCCGCCCGDR | |
| | SSPPVSPVRALDPPPGGVPLEAGLSLASLGSLGLS | |
| | EERKPSLFFQPAPGNAQSSSQTPLTVAMLSTGPTC | |
| | TSAS | |
| 10 | MERPARLCGLWALLLCAAGGRGGGVAAPTETQPPV | Canis lupus |
| | TNLSVSVENLCTVIWTWDPPEGASPNCTLRYFSHF | interleukin-13 receptor |
| | DNKQDKKIAPETHRSKEVPLNERICLQVGSQCSTN | subunit alpha-1 |
| | ESDNPSILVEKCTPPPEGDPESAVTELQCVWHNLS | |
| | YMKCTWLPGRNTSPDTNYTLYYWHSSLGKILQCED | |
| | IYREGQHIGCSFALTNLKDSSFEQHSVQIVVKDNA | |
| | GKIRPSFNIVPLTSHVKPDPPHIKRLFFQNGNLYV | |
| | QWKNPQNFYSRCLSYQVEVNNSQTETNDIFYVEEA | |
| | KCQNSEFEGNLEGTICFMVPGVLPDTLNTVRIRVR | |
| | TNKLCYEDDKLWSNWSQAMSIGENTDPTFYITMLL | |
| | ATPVIVAGAIIVLLLYLKRLKIIIFPPIPDPGKIF | |
| | KEMFGDQNDDTLHWRKYDIYEKQTKEETDSVVLIE | |
| | NLKKASQ | |
| 11 | MMTKCSSDRNVFKRKWFLFPASQYTERPIHQARPC | Felis catus |
| | EVPAVHLEPSPPWEVGLGLLNLESEFRKLGLRGRR | interleukin-13 receptor |
| | LAAAPPDSRAEAASQTQPPVTNLSVSVENLCTVIW | subunit alpha-1 |
| | TWDPPEGASPNCTLRYFSHFDNKQDKKIAPETHRS | |
| | KEVPLNERICLQVGSQCSTNESDNPSILVEKCTPP | |
| | PEGDPESAVTELQCVWHNLSYMKCTWLPGRNTSPD | |
| | TNYTLYYWHSSLGKILQCENIYREGQHIGCSFALT | |
| | NLKDSSFEQHSVQIVVKDNAGKIRPSFNIVPLTSH | |
| | VKPDPPHIKRLFFQNGNLYVQWKNPQNFYSRCLSY | |
| | QVEVNNSQTETHDIFYVEEAKCQNSEFEGNLEGTI | |
| | CFMVPGILPDTLNTVRIRVRTNKLCYEDDRLWSNW | |
| | SQAMSIGENTDPTFYITMLLATPVIVAGAIIVLLL | |
| | YLKRLKIIIFPPIPDPGKIFKEMFGDQNDDSLHWK | |
| | KYDIYEKQTKEETDSVVLIENASQ | |
| 12 | MYFLCLIWTESQPPVTNLSVSVENLCTVIWTWNPP | Equus caballus |
| | EGVSPNCSLWYFSHFGNKQDKKIAPETHRSKEVPL | interleukin-13 receptor |
| | NERICLQVGSQCSTNESDNPSILVEKCISPPEGDP | subunit alpha-1 |
| | ESAVTELQCVWHNLSYMKCTWLPGKNASPDTNYTL | |
| | YYWHSSLGKILQCEDIYREGQHIGCSFALTEVKDS | |
| | IFEQHSVQIMVKDNAGKIRPFFNIVPLTSHVKPDP | |
| | PHIKKLFFQNGDLYVQWKNPQNFYSRCLSYQVEVN | |
| | NSQTETRDIFSVEEAKCQNPEFEGDLEGTICFMVP | |
| | GVLPDTVNTVRIRVKTNKLCYEDDKLWSNWSQAMS | |
| | IGKKADPTFYIAMLLIIPVIVAGAIIVLLLYLKRL | |
| | KIIMFPPIPDPGKIFKEMFGDQNDDTLHWKKYDIY | |
| | EKQTKEETDSVVLIENLKRASQ | |
| 13 | TETQPPVTNLSVSVENLCTVIWTWDPPEGASPNCT | Exemplary Canis lupus |
| | LRYFSHFDNKQDKKIAPETHRSKEVPLNERICLQV | IL13RECD-IL4RECD-IgGA |
| | GSQCSTNESDNPSILVEKCTPPPEGDPESAVTELQ | Fc |
| | CVWHNLSYMKCTWLPGRNTSPDTNYTLYYWHSSLG | (without signal sequence) |
| | KILQCEDIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETND | |
| | IFYVEEAKCQNSEFEGNLEGTICFMVPGVLPDTLN | |
| | TVRIRVRTNKLCYEDDKLWSNWSQAMSIGENTDPT | |
| | GGGSGSGSVKVLHEPSCFSDYISTSVCQWKMDHPT | |
| | NCSAELRLSYQLDEMGSENHTCVPENREDSVCVCS | |
| | MPIDDAVEADVYQLDLWAGQQLLWSGSFQPSKHVK | |
| | PRTPGNLTVHPNISHTWLLMWTNPYPTENHLHSEL | |
| | TYMVNVSNDNDPEDFKVYNVTYMGPTLRLAASTLK | |
| | SGASYSARVRAWAQTYNSTWSDWSPSTTWLNYYEP | |
| | KRENGRVPRPPDCPKCPAPEMLGGPSVFIFPPKPK | |
| | DTLLIARTPEVTCVVVDLDPEDPEVQISWFVDGKQ | |
| | MQTAKTQPREEQFNGTYRVVSVLPIGHQDWLKGKQ | |
| | FTCKVNNKALPSPIERTISKARGQAHQPSVYVLPP | |
| | SREELSKNTVSLTCLIKDFFPPDIDVEWQSNGQQE | |
| | PESKYRTTPPQLDEDGSYFLYSKLSVDKSRWQRGD | |
| | TFICAVMHEALHNHYTQESLSHSPGK | |
| 14 | SGSVKVLHEPSCFSDYISTSVCQWKMDHPTNCSAE | Exemplary canine |
| | LRLSYQLDEMGSENHTCVPENREDSVCVCSMPIDD | IL4RECD-IL13RECD-IgGA |
| | AVEADVYQLDLWAGQQLLWSGSFQPSKHVKPRTPG | Fc (without signal sequence) |
| | NLTVHPNISHTWLLMWTNPYPTENHLHSELTYMVN | |
| | VSNDNDPEDFKVYNVTYMGPTLRLAASTLKSGASY | |
| | SARVRAWAQTYNSTWSDWSPSTTWLNYYEPGGGSG | |
| | TETQPPVTNLSVSVENLCTVIWTWDPPEGASPNCT | |
| | LRYFSHFDNKQDKKIAPETHRSKEVPLNERICLQV | |
| | GSQCSTNESDNPSILVEKCTPPPEGDPESAVTELQ | |
| | CVWHNLSYMKCTWLPGRNTSPDTNYTLYYWHSSLG | |
| | KILQCEDIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETND | |
| | IFYVEEAKCQNSEFEGNLEGTICFMVPGVLPDTLN | |
| | TVRIRVRTNKLCYEDDKLWSNWSQAMSIGENTDPT | |
| | FNECRCTDTPPCPVPEPLGGPSVLIFPPKPKDILR | |
| | ITRTPEVTCVVLDLGREDPEVQISWFVDGKEVHTA | |
| | KTQSREQQFNGTYRVVSVLPIEHQDWLTGKEFKCR | |
| | VNHIDLPSPIERTISKARGRAHKPSVYVLPPSPKE | |
| | LSSSDTVSITCLIKDFYPPDIDVEWQSNGQQEPER | |
| | KHRMTPPQLDEDGSYFLYSKLSVDKSRWQQGDPFT | |
| | CAVMHETLQNHYTDLSLSHSPGK | |
| 15 | SGSVKVLHEPSCFSDYISTSVCQWKMDHPTNCSAE | Exemplary canine |
| | LRLSYQLDFMGSENHTCVPENREDSVCVCSMPIDD | IL4RECD-IL13RECD-IgGB |
| | AVEADVYQLDLWAGQQLLWSGSFQPSKHVKPRTPG | Fc |
| | NLTVHPNISHTWLLMWTNPYPTENHLHSELTYMVN | (without signal sequence) |
| | VSNDNDPEDFKVYNVTYMGPTLRLAASTLKSGASY | |
| | SARVRAWAQTYNSTWSDWSPSTTWLNYYEPGGGSG | |
| | TETQPPVTNLSVSVENLCTVIWTWDPPEGASPNCT | |
| | LRYFSHFDNKQDKKIAPETHRSKEVPLNERICLQV | |
| | GSQCSTNESDNPSILVEKCTPPPEGDPESAVTELQ | |
| | CVWHNLSYMKCTWLPGRNTSPDTNYTLYYWHSSLG | |
| | KILQCEDIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETND | |
| | IFYVEEAKCQNSEFEGNLEGTICFMVPGVLPDTLN | |
| | TVRIRVRTNKLCYEDDKLWSNWSQAMSIGENTDPT | |
| | PKRENGRVPRPPDCPKCPAPEMLGGPSVFIFPPKP | |
| | KDTLLIARTPEVTCVVVDLDPEDPEVQISWFVDGK | |
| | QMQTAKTQPREEQFNGTYRVVSVLPIGHQDWLKGK | |
| | QFTCKVNNKALPSPIERTISKARGQAHQPSVYVLP | |
| | PSREELSKNTVSLTCLIKDFFPPDIDVEWQSNGQQ | |
| | EPESKYRTTPPQLDEDGSYFLYSKLSVDKSRWQRG | |
| | DTFICAVMHEALHNHYTQESLSHSPGK | |
| 16 | SGSVKVLHEPSCFSDYISTSVCQWKMDHPTNCSAE | Exemplary canine |
| | LRLSYQLDEMGSENHTCVPENREDSVCVCSMPIDD | IL4RECD-IL13RECD-IgGC |
| | AVEADVYQLDLWAGQQLLWSGSFQPSKHVKPRTPG | (without signal sequence) |
| | NLTVHPNISHTWLLMWTNPYPTENHLHSELTYMVN | |
| | VSNDNDPEDFKVYNVTYMGPTLRLAASTLKSGASY | |
| | SARVRAWAQTYNSTWSDWSPSTTWLNYYEPGGGSG | |
| | TETQPPVTNLSVSVENLCTVIWTWDPPEGASPNCT | |
| | LRYFSHFDNKQDKKIAPETHRSKEVPLNERICLQV | |
| | GSQCSTNESDNPSILVEKCTPPPEGDPESAVTELQ | |
| | CVWHNLSYMKCTWLPGRNTSPDTNYTLYYWHSSLG | |
| | KILQCEDIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETND | |
| | IFYVEEAKCQNSEFEGNLEGTICFMVPGVLPDTLN | |
| | TVRIRVRTNKLCYEDDKLWSNWSQAMSIGENTDPT | |
| | AKECECKCNCNNCPCPGCGLLGGPSVFIFPPKPKD | |
| | ILVTARTPTVTCVVVDLDPENPEVQISWFVDSKQV | |
| | QTANTQPREEQSNGTYRVVSVLPIGHQDWLSGKQF | |
| | KCKVNNKALPSPIEEIISKTPGQAHQPNVYVLPPS | |
| | RDEMSKNTVTLTCLVKDFFPPEIDVEWQSNGQQEP | |
| | ESKYRMTPPQLDEDGSYFLYSKLSVDKSRWQRGDT | |
| | FICAVMHEALHNHYTQISLSHSPGK | |
| 17 | SGSVKVLHEPSCFSDYISTSVCQWKMDHPTNCSAE | Exemplary canine |
| | LRLSYQLDFMGSENHTCVPENREDSVCVCSMPIDD | IL4RECD-IL13RECD-IgGD |
| | AVEADVYQLDLWAGQQLLWSGSFQPSKHVKPRTPG | Fc (without signal sequence) |
| | NLTVHPNISHTWLLMWTNPYPTENHLHSELTYMVN | |
| | VSNDNDPEDFKVYNVTYMGPTLRLAASTLKSGASY | |
| | SARVRAWAQTYNSTWSDWSPSTTWLNYYEPGGGSG | |
| | TETQPPVTNLSVSVENLCTVIWTWDPPEGASPNCT | |
| | LRYFSHFDNKQDKKIAPETHRSKEVPLNERICLQV | |
| | GSQCSTNESDNPSILVEKCTPPPEGDPESAVTELQ | |
| | CVWHNLSYMKCTWLPGRNTSPDTNYTLYYWHSSLG | |
| | KILQCEDIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETND | |
| | IFYVEEAKCQNSEFEGNLEGTICFMVPGVLPDTLN | |
| | TVRIRVRTNKLCYEDDKLWSNWSQAMSIGENTDPT | |
| | PKESTCKCISPCPVPESLGGPSVFIFPPKPKDILR | |
| | ITRTPEITCVVLDLGREDPEVQISWFVDGKEVHTA | |
| | KTQPREQQFNSTYRVVSVLPIEHQDWLTGKEFKCR | |
| | VNHIGLPSPIERTISKARGQAHQPSVYVLPPSPKE | |
| | LSSSDTVTLTCLIKDFFPPEIDVEWQSNGQPEPES | |
| | KYHTTAPQLDEDGSYFLYSKLSVDKSRWQQGDTFT | |
| | CAVMHEALQNHYTDLSLSHSPGK | |
| 18 | SGSVKVLRAPTCESDYFSTSVCQWNMDAPTNCSAE | Exemplary Feline |
| | LRLSYQLNEMGSENRTCVPENGEGAACACSMLMDD | IL4RECD-IL13RECD |
| | FVEADVYQLHLWAGTQLLWSGSFKPSSHVKPRAPG | (without signal sequence) |
| | NLTVHPNVSHTWLLRWSNPYPPENHLHAELTYMVN | |
| | ISSEDDPTDVSVCASGFLCHLLGLRRVETGAPGAR | |
| | LPPWLCAPRPRRVPGSQCAVISCCRWVLIALTSRG | |
| | GRWRLTPGLRSQTRYVSVAEGLFGATPRVLCPGTQ | |
| | AGLASAAREQMSPDPSAFHSIDYEPGGGSGSQTQP | |
| | PVTNLSVSVENLCTVIWTWDPPEGASPNCTLRYFS | |
| | HFDNKQDKKIAPETHRSKEVPLNERICLQVGSQCS | |
| | TNESDNPSILVEKCTPPPEGDPESAVTELQCVWHN | |
| | LSYMKCTWLPGRNTSPDTNYTLYYWHSSLGKILQC | |
| | ENIYREGQHIGCSFALTNLKDSSFEQHSVQIVVKD | |
| | NAGKIRPSFNIVPLTSHVKPDPPHIKRLFFQNGNL | |
| | YVQWKNPQNFYSRCLSYQVEVNNSQTETHDIFYVE | |
| | EAKCQNSEFEGNLEGTICFMVPGILPDTLNTVRIR | |
| | VRTNKLCYEDDRLWSNWSQAMSIGENTDPT | |
| 19 | SGSVKVLHLTACFSDYISASTCEWKMDRPTNCSAQ | Exemplary equine |
| | LRLSYQLNDEFSDNLTCIPENREDEVCVCRMLMDN | IL4RECD-IL13RECD |
| | IVSEDVYELDLWAGNQLLWNSSFKPSRHVKPRAPQ | (without signal sequence) |
| | NLTVHAISHTWLLTWSNPYPLKNHLWSELTYLVNI | |
| | SKEDDPTDFKIYNVTYMDPTLRVTASTLKSRATYS | |
| | ARVKARAQNYNSTWSEWSPSTTWHNYYEQPGGGSG | |
| | TESQPPVTNLSVSVENLCTVIWTWNPPEGVSPNCS | |
| | LWYFSHFGNKQDKKIAPETHRSKEVPLNERICLQV | |
| | GSQCSTNESDNPSILVEKCISPPEGDPESAVTELQ | |
| | CVWHNLSYMKCTWLPGKNASPDTNYTLYYWHSSLG | |
| | KILQCEDIYREGQHIGCSFALTEVKDSIFEQHSVQ | |
| | IMVKDNAGKIRPFFNIVPLTSHVKPDPPHIKKLFF | |
| | QNGDLYVQWKNPQNFYSRCLSYQVEVNNSQTETRD | |
| | IFSVEEAKCQNPEFEGDLEGTICFMVPGVLPDTVN | |
| | TVRIRVKTNKLCYEDDKLWSNWSQAMSIGKKADPT | |
| 20 | MAVLGLLFCLVTFPSCVLSTETQPPVTNLSVSVEN | Exemplary canine |
| | LCTVIWTWDPPEGASPNCTLRYFSHFDNKQDKKIA | IL13RECD-IL4RECD-IgGB |
| | PETHRSKEVPLNERICLQVGSQCSTNESDNPSILV | Fc |
| | EKCTPPPEGDPESAVTELQCVWHNLSYMKCTWLPG | (with signal sequence) |
| | RNTSPDTNYTLYYWHSSLGKILQCEDIYREGQHIG | |
| | CSFALTNLKDSSFEQHSVQIVVKDNAGKIRPSFNI | |
| | VPLTSHVKPDPPHIKRLFFQNGNLYVQWKNPQNFY | |
| | SRCLSYQVEVNNSQTETNDIFYVEEAKCQNSEFEG | |
| | NLEGTICFMVPGVLPDTLNTVRIRVRTNKLCYEDD | |
| | KLWSNWSQAMSIGENTDPTGGGSGSGSVKVLHEPS | |
| | CFSDYISTSVCQWKMDHPTNCSAELRLSYQLDEMG | |
| | SENHTCVPENREDSVCVCSMPIDDAVEADVYQLDL | |
| | WAGQQLLWSGSFQPSKHVKPRTPGNLTVHPNISHT | |
| | WLLMWTNPYPTENHLHSELTYMVNVSNDNDPEDFK | |
| | VYNVTYMGPTLRLAASTLKSGASYSARVRAWAQTY | |
| | NSTWSDWSPSTTWLNYYEPKRENGRVPRPPDCPKC | |
| | PAPEMLGGPSVFIFPPKPKDTLLIARTPEVTCVVV | |
| | DLDPEDPEVQISWFVDGKQMQTAKTQPREEQENGT | |
| | YRVVSVLPIGHQDWLKGKQFTCKVNNKALPSPIER | |
| | TISKARGQAHQPSVYVLPPSREELSKNTVSLTCLI | |
| | KDFFPPDIDVEWQSNGQQEPESKYRTTPPQLDEDG | |
| | SYFLYSKLSVDKSRWQRGDTFICAVMHEALHNHYT | |
| | QESLSHSPGK | |
| 21 | MAVLGLLFCLVTFPSCVLSSGSVKVLHEPSCFSDY | Exemplary canine |
| | ISTSVCQWKMDHPTNCSAELRLSYQLDEMGSENHT | IL4RECD-IL13RECD-IgGB |
| | CVPENREDSVCVCSMPIDDAVEADVYQLDLWAGQQ | Fc |
| | LLWSGSFQPSKHVKPRTPGNLTVHPNISHTWLLMW | (with signal sequence) |
| | TNPYPTENHLHSELTYMVNVSNDNDPEDFKVYNVT | |
| | YMGPTLRLAASTLKSGASYSARVRAWAQTYNSTWS | |
| | DWSPSTTWLNYYEPGGGSGTETQPPVTNLSVSVEN | |
| | LCTVIWTWDPPEGASPNCTLRYFSHFDNKQDKKIA | |
| | PETHRSKEVPLNERICLQVGSQCSTNESDNPSILV | |
| | EKCTPPPEGDPESAVTELQCVWHNLSYMKCTWLPG | |
| | RNTSPDTNYTLYYWHSSLGKILQCEDIYREGQHIG | |
| | CSFALTNLKDSSFEQHSVQIVVKDNAGKIRPSFNI | |
| | VPLTSHVKPDPPHIKRLFFQNGNLYVQWKNPQNFY | |
| | SRCLSYQVEVNNSQTETNDIFYVEEAKCQNSEFEG | |
| | NLEGTICFMVPGVLPDTLNTVRIRVRTNKLCYEDD | |
| | KLWSNWSQAMSIGENTDPTPKRENGRVPRPPDCPK | |
| | CPAPEMLGGPSVFIFPPKPKDTLLIARTPEVTCVV | |
| | VDLDPEDPEVQISWFVDGKQMQTAKTQPREEQENG | |
| | TYRVVSVLPIGHQDWLKGKQFTCKVNNKALPSPIE | |
| | RTISKARGQAHQPSVYVLPPSREELSKNTVSLTCL | |
| | IKDFFPPDIDVEWQSNGQQEPESKYRTTPPQLDED | |
| | GSYFLYSKLSVDKSRWQRGDTFICAVMHEALHNHY | |
| | TQESLSHSPGK | |
| 22 | TETQPPVTNLSVSVENLCTVIWTWDPPEGASPNCT | Exemplary canine IL13R |
| | LRYFSHFDNKQDKKIAPETHRSKEVPLNERICLQV | extracellular domain |
| | GSQCSTNESDNPSILVEKCTPPPEGDPESAVTELQ | (without signal sequence) |
| | CVWHNLSYMKCTWLPGRNTSPDTNYTLYYWHSSLG | |
| | KILQCEDIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETND | |
| | IFYVEEAKCQNSEFEGNLEGTICFMVPGVLPDTLN | |
| | TVRIRVRTNKLCYEDDKLWSNWSQAMSIGENTDPT | |
| 23 | SGSVKVLHEPSCFSDYISTSVCQWKMDHPTNCSAE | Exemplary canine IL4R |
| | LRLSYQLDEMGSENHTCVPENREDSVCVCSMPIDD | extracellular domain (ECD; |
| | AVEADVYQLDLWAGQQLLWSGSFQPSKHVKPRTPG | without signal sequence) |
| | NLTVHPNISHTWLLMWTNPYPTENHLHSELTYMVN | |
| | VSNDNDPEDFKVYNVTYMGPTLRLAASTLKSGASY | |
| | SARVRAWAQTYNSTWSDWSPSTTWLNYYEP | |
| 163 | SGSVKVLHEPSCFSDYISTSVCQWKMDHPTNCSAE | Exemplary canine IL4R |
| | LRLSYQLDFMGSENHTCVPENREDSVCVCSMPIDD | extracellular domain (ECD; |
| | AVEADVYQLDLWAGQQLLWSGSFQPSKHVKPRTPG | without signal sequence) |
| | NLTVHPNISHTWLLMWTNPYPTENHLHSELTYMVN | |
| | VSNDNDPEDFKVYNVTYMGPTLRLAASTLKSGASY | |
| | SARVRAWAQTYNSTWSDWSPSTTWLNYYE | |
| 24 | SQTQPPVTNLSVSVENLCTVIWTWDPPEGASPNCT | Exemplary feline IL13R |
| | LRYFSHFDNKQDKKIAPETHRSKEVPLNERICLQV | extracellular domain (ECD; |
| | GSQCSTNESDNPSILVEKCTPPPEGDPESAVTELQ | without signal sequence) |
| | CVWHNLSYMKCTWLPGRNTSPDTNYTLYYWHSSLG | |
| | KILQCENIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETHD | |
| | IFYVEEAKCQNSEFEGNLEGTICFMVPGILPDTLN | |
| | TVRIRVRTNKLCYEDDRLWSNWSQAMSIGENTDPT | |
| 25 | SGSVKVLRAPTCESDYFSTSVCQWNMDAPTNCSAE | Exemplary feline IL4R |
| | LRLSYQLNFMGSENRTCVPENGEGAACACSMLMDD | extracellular domain (ECD; |
| | FVEADVYQLHLWAGTQLLWSGSFKPSSHVKPRAPG | without signal sequence) |
| | NLTVHPNVSHTWLLRWSNPYPPENHLHAELTYMVN | |
| | ISSEDDPTDVSVCASGFLCHLLGLRRVETGAPGAR | |
| | LPPWLCAPRPRRVPGSQCAVISCCRWVLIALTSRG | |
| | GRWRLTPGLRSQTRYVSVAEGLEGATPRVLCPGTQ | |
| | AGLASAAREQMSPDPSAFHSIDYEP | |
| 26 | TESQPPVTNLSVSVENLCTVIWTWNPPEGVSPNCS | Exemplary equine IL13R |
| | LWYFSHFGNKQDKKIAPETHRSKEVPLNERICLQV | extracellular domain (ECD; |
| | GSQCSTNESDNPSILVEKCISPPEGDPESAVTELQ | without signal sequence) |
| | CVWHNLSYMKCTWLPGKNASPDTNYTLYYWHSSLG | |
| | KILQCEDIYREGQHIGCSFALTEVKDSIFEQHSVQ | |
| | IMVKDNAGKIRPFFNIVPLTSHVKPDPPHIKKLFF | |
| | QNGDLYVQWKNPQNFYSRCLSYQVEVNNSQTETRD | |
| | IFSVEEAKCQNPEFEGDLEGTICFMVPGVLPDTVN | |
| | TVRIRVKTNKLCYEDDKLWSNWSQAMSIGKKADPT | |
| 27 | SGSVKVLHLTACFSDYISASTCEWKMDRPTNCSAQ | Exemplary equine IL4R |
| | LRLSYQLNDEFSDNLTCIPENREDEVCVCRMLMDN | extracellular domain (ECD; |
| | IVSEDVYELDLWAGNQLLWNSSFKPSRHVKPRAPQ | without signal sequence) |
| | NLTVHAISHTWLLTWSNPYPLKNHLWSELTYLVNI | |
| | SKEDDPTDFKIYNVTYMDPTLRVTASTLKSRATYS | |
| | ARVKARAQNYNSTWSEWSPSTTWHNYYEQP | |
| 28 | SQTQPPVTNLSVSVENLCTVIWTWDPPEGASPNCT | Exemplary feline |
| | LRYFSHFDNKQDKKIAPETHRSKEVPLNERICLQV | IL13RECD-IL4RECD-IgG2 |
| | GSQCSTNESDNPSILVEKCTPPPEGDPESAVTELQ | Fc (without signal sequence) |
| | CVWHNLSYMKCTWLPGRNTSPDTNYTLYYWHSSLG | |
| | KILQCENIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETHD | |
| | IFYVEEAKCQNSEFEGNLEGTICFMVPGILPDTLN | |
| | TVRIRVRTNKLCYEDDRLWSNWSQAMSIGENTDPT | |
| | GGGSGSSGSVKVLRAPTCFSDYFSTSVCQWNMDAP | |
| | TNCSAELRLSYQLNEMGSENRTCVPENGEGAACAC | |
| | SMLMDDFVEADVYQLHLWAGTQLLWSGSFKPSSHV | |
| | KPRAPGNLTVHPNVSHTWLLRWSNPYPPENHLHAE | |
| | LTYMVNISSEDDPTDVSVCASGFLCHLLGLRRVET | |
| | GAPGARLPPWLCAPRPRRVPGSQCAVISCCRWVLI | |
| | ALTSRGGRWRLTPGLRSQTRYVSVAEGLFGATPRV | |
| | LCPGTQAGLASAAREQMSPDPSAFHSIDYEPSPKT | |
| | ASTIESKTGECPKCPVPEIPGAPSVFIFPPKPKDT | |
| | LSISRTPEVTCLVVDLGPDDSNVQITWFVDNTEMH | |
| | TAKTRPREEQFNSTYRVVSVLPILHQDWLKGKEFK | |
| | CKVNSKSLPSAMERTISKAKGQPHEPQVYVLPPTQ | |
| | EELSENKVSVTCLIKGFHPPDIAVEWEITGQPEPE | |
| | NNYQTTPPQLDSDGTYFLYSRLSVDRSHWQRGNTY | |
| | TCSVSHEALHSHHTQKSLTQSPGK | |
| 29 | SGSVKVLRAPTCFSDYFSTSVCQWNMDAPTNCSAE | Exemplary feline |
| | LRLSYQLNEMGSENRTCVPENGEGAACACSMLMDD | IL4RECD-IL13RECD-IgG2 |
| | FVEADVYQLHLWAGTQLLWSGSFKPSSHVKPRAPG | (without signal sequence) |
| | NLTVHPNVSHTWLLRWSNPYPPENHLHAELTYMVN | |
| | ISSEDDPTDVSVCASGFLCHLLGLRRVETGAPGAR | |
| | LPPWLCAPRPRRVPGSQCAVISCCRWVLIALTSRG | |
| | GRWRLTPGLRSQTRYVSVAEGLFGATPRVLCPGTQ | |
| | AGLASAAREQMSPDPSAFHSIDYEPGGGSGSSQTQ | |
| | PPVTNLSVSVENLCTVIWTWDPPEGASPNCTLRYF | |
| | SHFDNKQDKKIAPETHRSKEVPLNERICLQVGSQC | |
| | STNESDNPSILVEKCTPPPEGDPESAVTELQCVWH | |
| | NLSYMKCTWLPGRNTSPDTNYTLYYWHSSLGKILQ | |
| | CENIYREGQHIGCSFALTNLKDSSFEQHSVQIVVK | |
| | DNAGKIRPSFNIVPLTSHVKPDPPHIKRLFFQNGN | |
| | LYVQWKNPQNFYSRCLSYQVEVNNSQTETHDIFYV | |
| | EEAKCQNSEFEGNLEGTICFMVPGILPDTLNTVRI | |
| | RVRTNKLCYEDDRLWSNWSQAMSIGENTDPTSPKT | |
| | ASTIESKTGECPKCPVPEIPGAPSVFIFPPKPKDT | |
| | LSISRTPEVTCLVVDLGPDDSNVQITWFVDNTEMH | |
| | TAKTRPREEQFNSTYRVVSVLPILHQDWLKGKEFK | |
| | CKVNSKSLPSAMERTISKAKGQPHEPQVYVLPPTQ | |
| | EELSENKVSVTCLIKGFHPPDIAVEWEITGQPEPE | |
| | NNYQTTPPQLDSDGTYFLYSRLSVDRSHWQRGNTY | |
| | TCSVSHEALHSHHTQKSLTQSPGK | |
| 30 | TESQPPVTNLSVSVENLCTVIWTWNPPEGVSPNCS | Exemplary equine |
| | LWYFSHFGNKQDKKIAPETHRSKEVPLNERICLQV | IL13RECD-IL4RECD-IgG2 |
| | GSQCSTNESDNPSILVEKCISPPEGDPESAVTELQ | Fc (without signal sequence) |
| | CVWHNLSYMKCTWLPGKNASPDTNYTLYYWHSSLG | |
| | KILQCEDIYREGQHIGCSFALTEVKDSIFEQHSVQ | |
| | IMVKDNAGKIRPFFNIVPLTSHVKPDPPHIKKLFF | |
| | QNGDLYVQWKNPQNFYSRCLSYQVEVNNSQTETRD | |
| | IFSVEEAKCQNPEFEGDLEGTICFMVPGVLPDTVN | |
| | TVRIRVKTNKLCYEDDKLWSNWSQAMSIGKKADPT | |
| | GGGSGSSGSVKVLHLTACFSDYISASTCEWKMDRP | |
| | TNCSAQLRLSYQLNDEFSDNLTCIPENREDEVCVC | |
| | RMLMDNIVSEDVYELDLWAGNQLLWNSSFKPSRHV | |
| | KPRAPQNLTVHAISHTWLLTWSNPYPLKNHLWSEL | |
| | TYLVNISKEDDPTDFKIYNVTYMDPTLRVTASTLK | |
| | SRATYSARVKARAQNYNSTWSEWSPSTTWHNYYEQ | |
| | PDMSKCPKCPAPELLGGPSVFIFPPNPKDTLMISR | |
| | TPVVTCVVVNLSDQYPDVQFSWYVDNTEVHSAITK | |
| | QREAQFNSTYRVVSVLPIQHQDWLSGKEFKCSVTN | |
| | VGVPQPISRAISRGKGPSRVPQVYVLPPHPDELAK | |
| | SKVSVTCLVKDFYPPDISVEWQSNRWPELEGKYST | |
| | TPAQLDGDGSYFLYSKLSLETSRWQQVESFTCAVM | |
| | HEALHNHYTKTDISESLGK | |
| 31 | SGSVKVLHLTACFSDYISASTCEWKMDRPTNCSAQ | Exemplary equine |
| | LRLSYQLNDEFSDNLTCIPENREDEVCVCRMLMDN | IL4RECD-IL13RECD-IgG2 |
| | IVSEDVYELDLWAGNQLLWNSSFKPSRHVKPRAPQ | Fc (without signal sequence) |
| | NLTVHAISHTWLLTWSNPYPLKNHLWSELTYLVNI | |
| | SKEDDPTDEKIYNVTYMDPTLRVTASTLKSRATYS | |
| | ARVKARAQNYNSTWSEWSPSTTWHNYYEQPGGGSG | |
| | STESQPPVTNLSVSVENLCTVIWTWNPPEGVSPNC | |
| | SLWYFSHFGNKQDKKIAPETHRSKEVPLNERICLQ | |
| | VGSQCSTNESDNPSILVEKCISPPEGDPESAVTEL | |
| | QCVWHNLSYMKCTWLPGKNASPDTNYTLYYWHSSL | |
| | GKILQCEDIYREGQHIGCSFALTEVKDSIFEQHSV | |
| | QIMVKDNAGKIRPFFNIVPLTSHVKPDPPHIKKLF | |
| | FQNGDLYVQWKNPQNFYSRCLSYQVEVNNSQTETR | |
| | DIFSVEEAKCQNPEFEGDLEGTICFMVPGVLPDTV | |
| | NTVRIRVKTNKLCYEDDKLWSNWSQAMSIGKKADP | |
| | TDMSKCPKCPAPELLGGPSVFIFPPNPKDTLMISR | |
| | TPVVTCVVVNLSDQYPDVQFSWYVDNTEVHSAITK | |
| | QREAQFNSTYRVVSVLPIQHQDWLSGKEFKCSVIN | |
| | VGVPQPISRAISRGKGPSRVPQVYVLPPHPDELAK | |
| | SKVSVTCLVKDFYPPDISVEWQSNRWPELEGKYST | |
| | TPAQLDGDGSYFLYSKLSLETSRWQQVESFTCAVM | |
| | HEALHNHYTKTDISESLGK | |
| 32 | QPPVTNLSVSVENLCTVIWTWDPPEGASPNCTLRY | Exemplary canine mini- |
| | FSHFDNKQDKKIAPETHRSKEVPLNERICLQVGSQ | IL13R ECD |
| | CSTNESDNPSILVEKCTPPPEGDPESAVTELQCVW | |
| | HNLSYMKCTWLPGRNTSPDTNYTLYYWHSSLGKIL | |
| | QCEDIYREGQHIGCSFALTNLKDSSFEQHSVQIVV | |
| | KDNAGKIRPSFNIVPLTSHVKPDPPHIKRLFFQNG | |
| | NLYVQWKNPQNFYSRCLSYQVEVNNSQTETNDIFY | |
| | VEEAKCQNSEFEGNLEGTICFMVPGVLPDTLNTVR | |
| | IRVRTNKLCYEDDKLWSNWSQAMSI | |
| 33 | KVLHEPSCFSDYISTSVCQWKMDHPTNCSAELRLS | Exemplary canine mini-IL4R |
| | YQLDEMGSENHTCVPENREDSVCVCSMPIDDAVEA | ECD |
| | DVYQLDLWAGQQLLWSGSFQPSKHVKPRTPGNLTV | |
| | HPNISHTWLLMWTNPYPTENHLHSELTYMVNVSND | |
| | NDPEDFKVYNVTYMGPTLRLAASTLKSGASYSARV | |
| | RAWAQTYNS | |
| 34 | QPPVTNLSVSVENLCTVIWTWDPPEGASPNCTLRY | Exemplary feline mini- |
| | FSHFDNKQDKKIAPETHRSKEVPLNERICLQVGSQ | IL 13R ECD |
| | CSTNESDNPSILVEKCTPPPEGDPESAVTELQCVW | |
| | HNLSYMKCTWLPGRNTSPDTNYTLYYWHSSLGKIL | |
| | QCENIYREGQHIGCSFALTNLKDSSFEQHSVQIVV | |
| | KDNAGKIRPSFNIVPLTSHVKPDPPHIKRLFFQNG | |
| | NLYVQWKNPQNFYSRCLSYQVEVNNSQTETHDIFY | |
| | VEEAKCQNSEFEGNLEGTICFMVPGILPDTLNTVR | |
| | IRVRTNKLCYEDDRLWSNWSQAMSI | |
| 35 | KVLRAPTCFSDYFSTSVCQWNMDAPTNCSAELRLS | Exemplary feline mini-IL4R |
| | YQLNFMGSENRTCVPENGEGAACACSMLMDDFVEA | ECD |
| | DVYQLHLWAGTQLLWSGSFKPSSHVKPRAPGNLTV | |
| | HPNVSHTWLLRWSNPYPPENHLHAELTYMVNISSE | |
| | DDPTDVSVCASGFLCHLLGLRRVETGAPGARLPPW | |
| | LCAPRPRRVPGSQCAVISCCRWVLIALTSRGGRWR | |
| | LTPGLRSQTRYVSVAEGLFGATPRVLCPGTQAGLA | |
| | SAAREQMSPDPSAFHSIDYEP | |
| 36 | QPPVTNLSVSVENLCTVIWTWNPPEGVSPNCSLWY | Exemplary equine mini- |
| | FSHFGNKQDKKIAPETHRSKEVPLNERICLQVGSQ | IL 13R ECD |
| | CSTNESDNPSILVEKCISPPEGDPESAVTELQCVW | |
| | HNLSYMKCTWLPGKNASPDTNYTLYYWHSSLGKIL | |
| | QCEDIYREGQHIGCSFALTEVKDSIFEQHSVQIMV | |
| | KDNAGKIRPFFNIVPLTSHVKPDPPHIKKLFFQNG | |
| | DLYVQWKNPQNFYSRCLSYQVEVNNSQTETRDIES | |
| | VEEAKCQNPEFEGDLEGTICFMVPGVLPDTVNTVR | |
| | IRVKTNKLCYEDDKLWSNWSQAMSI | |
| 37 | KVLHLTACFSDYISASTCEWKMDRPTNCSAQLRLS | Exemplary equine mini-IL4R |
| | YQLNDEFSDNLTCIPENREDEVCVCRMLMDNIVSE | ECD |
| | DVYELDLWAGNQLLWNSSFKPSRHVKPRAPQNLTV | |
| | HAISHTWLLTWSNPYPLKNHLWSELTYLVNISKED | |
| | DPTDFKIYNVTYMDPTLRVTASTLKSRATYSARVK | |
| | ARAQNYNSTWSEWSPSTTWHNYYEQP | |
| 38 | PVPEPLGGPSVLIFPPKPKDILRITRTPEVTCVVL | Exemplary wild-type canine |
| | DLGREDPEVQISWFVDGKEVHTAKTQSREQQENGT | IgG-A Fc |
| | YRVVSVLPIEHQDWLTGKEFKCRVNHIDLPSPIER | |
| | TISKARGRAHKPSVYVLPPSPKELSSSDTVSITCL | |
| | IKDFYPPDIDVEWQSNGQQEPERKHRMTPPQLDED | |
| | GSYFLYSKLSVDKSRWQQGDPFTCAVMHETLQNHY | |
| | TDLSLSHSPGK | |
| 39 | PAPEMLGGPSVFIFPPKPKDTLLIARTPEVTCVVV | Exemplary wild-type canine |
| | DLDPEDPEVQISWFVDGKQMQTAKTQPREEQENGT | IgG-B Fc |
| | YRVVSVLPIGHQDWLKGKQFTCKVNNKALPSPIER | Protein A+ |
| | TISKARGQAHQPSVYVLPPSREELSKNTVSLTCLI | C1q+ |
| | KDFFPPDIDVEWQSNGQQEPESKYRTTPPQLDEDG | CD16+ |
| | SYFLYSKLSVDKSRWQRGDTFICAVMHEALHNHYT | |
| | QESLSHSPGK | |
| 150 | PKRENGRVPRPPDCPKCPAPEMLGGPSVFIFPPKP | Exemplary wild-type canine |
| | KDTLLIARTPEVTCVVVDLDPEDPEVQISWFVDGK | IgG-B Fc with hinge |
| | QMQTAKTQPREEQFNGTYRVVSVLPIGHQDWLKGK | Protein A+ |
| | QFTCKVNNKALPSPIERTISKARGQAHQPSVYVLP | C1q+ |
| | PSREELSKNTVSLTCLIKDFFPPDIDVEWQSNGQQ | CD16 + |
| | EPESKYRTTPPQLDEDGSYFLYSKLSVDKSRWQRG | |
| | DTFICAVMHEALHNHYTQESLSHSPGK | |
| 40 | PGCGLLGGPSVFIFPPKPKDILVTARTPTVTCVVV | Exemplary wild-type canine |
| | DLDPENPEVQISWFVDSKQVQTANTQPREEQSNGT | IgG-C Fc |
| | YRVVSVLPIGHQDWLSGKQFKCKVNNKALPSPIEE | |
| | IISKTPGQAHQPNVYVLPPSRDEMSKNTVTLTCLV | |
| | KDFFPPEIDVEWQSNGQQEPESKYRMTPPQLDEDG | |
| | SYFLYSKLSVDKSRWQRGDTFICAVMHEALHNHYT | |
| | QISLSHSPGK | |
| 41 | PVPESLGGPSVFIFPPKPKDILRITRTPEITCVVL | Exemplary wild-type canine |
| | DLGREDPEVQISWFVDGKEVHTAKTQPREQQENST | IgG-D Fc |
| | YRVVSVLPIEHQDWLTGKEFKCRVNHIGLPSPIER | |
| | TISKARGQAHQPSVYVLPPSPKELSSSDTVTLTCL | |
| | IKDFFPPEIDVEWQSNGQPEPESKYHTTAPQLDED | |
| | GSYFLYSKLSVDKSRWQQGDTFTCAVMHEALQNHY | |
| | TDLSLSHSPGK | |
| 42 | RKTDHPPGPKPCDCPKCPPPEMLGGPSIFIFPPKP | Exemplary wild-type feline |
| | KDTLSISRTPEVTCLVVDLGPDDSDVQITWFVDNT | IgG1a Fc |
| | QVYTAKTSPREEQFNSTYRVVSVLPILHQDWLKGK | |
| | EFKCKVNSKSLPSPIERTISKAKGQPHEPQVYVLP | |
| | PAQEELSENKVSVTCLIKSFHPPDIAVEWEITGQP | |
| | EPENNYRTTPPQLDSDGTYFVYSKLSVDRSHWQRG | |
| | NTYTCSVSHEALHSHHTQKSLTQSPGK | |
| 43 | RKTDHPPGPKTGEGPKCPPPEMLGGPSIFIFPPKP | Exemplary wild-type feline |
| | KDTLSISRTPEVTCLVVDLGPDDSDVQITWFVDNT | IgG1a Fc |
| | QVYTAKTSPREEQFNSTYRVVSVLPILHQDWLKGK | |
| | EFKCKVNSKSLPSPIERTISKAKGQPHEPQVYVLP | |
| | PAQEELSENKVSVTCLIKSFHPPDIAVEWEITGQP | |
| | EPENNYRTTPPQLDSDGTYFVYSKLSVDRSHWQRG | |
| | NTYTCSVSHEALHSHHTQKSLTQSPGK | |
| 44 | RKTDHPPGPKPCDCPKCPPPEMLGGPSIFIFPPKP | Exemplary wild-type feline |
| | KDTLSISRTPEVTCLVVDLGPDDSDVQITWFVDNT | IgG1b Fc |
| | QVYTAKTSPREEQFNSTYRVVSVLPILHQDWLKGK | |
| | EFKCKVNSKSLPSPIERTISKDKGQPHEPQVYVLP | |
| | PAQEELSENKVSVTCLIEGFYPSDIAVEWEITGQP | |
| | EPENNYRTTPPQLDSDGTYFLYSRLSVDRSRWQRG | |
| | NTYTCSVSHEALHSHHTQKSLTQSPGK | |
| 45 | RKTDHPPGPKTGEGPKCPPPEMLGGPSIFIFPPKP | Exemplary wild-type feline |
| | KDTLSISRTPEVTCLVVDLGPDDSDVQITWFVDNT | IgG1b Fc |
| | QVYTAKTSPREEQFNSTYRVVSVLPILHQDWLKGK | |
| | EFKCKVNSKSLPSPIERTISKDKGQPHEPQVYVLP | |
| | PAQEELSENKVSVTCLIEGFYPSDIAVEWEITGQP | |
| | EPENNYRTTPPQLDSDGTYFLYSRLSVDRSRWQRG | |
| | NTYTCSVSHEALHSHHTQKSLTQSPGK | |
| 46 | PKTASTIESKTGEGPKCPVPEIPGAPSVFIFPPKP | Exemplary wild-type feline |
| | KDTLSISRTPEVTCLVVDLGPDDSNVQITWFVDNT | IgG2 Fc |
| | EMHTAKTRPREEQFNSTYRVVSVLPILHQDWLKGK | |
| | EFKCKVNSKSLPSAMERTISKAKGQPHEPQVYVLP | |
| | PTQEELSENKVSVTCLIKGFHPPDIAVEWEITGQP | |
| | EPENNYQTTPPQLDSDGTYFLYSRLSVDRSHWQRG | |
| | NTYTCSVSHEALHSHHTQKSLTQSPGK | |
| 47 | GPSVFIFPPNPKDTLMITRTPEVTCVVVDVSQENP | Exemplary wild-type equine |
| | DVKFNWYMDGVEVRTATTRPKEEQFNSTYRVVSVL | IgG1 Fc |
| | RIQHQDWLSGKEFKCKVNNQALPQPIERTITKTKG | |
| | RSQEPQVYVLAPHPDELSKSKVSVTCLVKDFYPPE | |
| | INIEWQSNGQPELETKYSTTQAQQDSDGSYFLYSK | |
| | LSVDRNRWQQGTTFTCGVMHEALHNHYTQKNVSKN | |
| | PGK | |
| 48 | GPSVFIFPPNPKDALMISRTPVVTCVVVNLSDQYP | Exemplary wild-type equine |
| | DVQFSWYVDNTEVHSAITKQREAQFNSTYRVVSVL | IgG2 Fc |
| | PIQHQDWLSGKEFKCSVTNVGVPQPISRAISRGKG | |
| | PSRVPQVYVLPPHPDELAKSKVSVTCLVKDFYPPD | |
| | ISVEWQSNRWPELEGKYSTTPAQLDGDGSYFLYSK | |
| | LSLETSRWQQVESFTCAVMHEALHNHFTKTDISES | |
| | LGK | |
| 49 | GPSVFIFPPKPKDVLMITRMPEVTCLVVDVSHDSS | Exemplary wild-type equine |
| | DVLFTWYVDGTEVKTAKTMPNEEQNNSTYRVVSVL | IgG3 Fc |
| | RIQHQDWLNGKKFKCKVNNQALPAPVERTISKATG | |
| | QTRVPQVYVLAPHPDELSKNKVSVTCLVKDFYPPD | |
| | ITVEWQSNEHPEPEGKYRTTEAQKDSDGSYFLYSK | |
| | LTVEKDRWQQGTTFTCVVMHEALHNHVMQKNISKN | |
| | PGK | |
| 50 | GPSVFIFPPKPKDVLMISRTPTVTCVVVDVGHDEP | Exemplary wild-type equine |
| | DVQFNWYVDGVETHTATTEPKQEQFNSTYRVVSVL | IgG4 Fc |
| | PIQHKDWLSGKEFKCKVNNKALPAPVERTISAPTG | |
| | QPREPQVYVLAPHRDELSKNKVSVTCLVKDFYPPD | |
| | IDIEWKSNGQPEPETKYSTTPAQLDSDGSYFLYSK | |
| | LTVETNRWQQGTTFTCAVMHEALHNHYTEKSVSKS | |
| | PGK | |
| 51 | GPSVFIFPPKPKDVLMISRKPEVTCVVVDLGHDDP | Exemplary wild-type equine |
| | DVQFTWFVDGVETHTATTEPKEEQENSTYRVVSVL | IgG5 Fc |
| | PIQHQDWLSGKEFKCSVTSKALPAPVERTISKAKG | |
| | QLRVPQVYVLAPHPDELAKNTVSVTCLVKDFYPPE | |
| | IDVEWQSNEHPEPEGKYSTTPAQLNSDGSYFLYSK | |
| | LSVETSRWKQGESFTCGVMHEAVENHYTQKNVSHS | |
| | PGK | |
| 52 | GRPSVFIFPPNPKDTLMISRTPEVTCVVVDVSQE | Exemplary wild-type equine |
| | NPDVKFNWYVDGVEAHTATTKAKEKQDNSTYRVV | IgG6 Fc |
| | SVLPIQHQDWRRGKEFKCKVNNRALPAPVERTIT | |
| | KAKGELQDPKVYILAPHREEVTKNTVSVTCLVKD | |
| | FYPPDINVEWQSNEEPEPEVKYSTTPAQLDGDGS | |
| | YFLYSKLTVETDRWEQGESFTCVVMHEAIRHTYR | |
| | QKSITNFPGK | |
| 53 | GPSVFIFPPKPKDVLMISRTPTVTCVVVDVGHDFP | Exemplary wild-type equine |
| | DVQFNWYVDGVETHTATTEPKQEQNNSTYRVVSIL | IgG7 Fc |
| | AIQHKDWLSGKEFKCKVNNQALPAPVQKTISKPTG | |
| | QPREPQVYVLAPHPDELSKNKVSVTCLVKDFYPPD | |
| | IDIEWKSNGQPEPETKYSTTPAQLDGDGSYFLYSK | |
| | LTVETNRWQQGTTFTCAVMHEALHNHYTEKSVSKS | |
| | PGK | |
| 54 | PVPEPLGGPSVLIFPPKPKDILRITRTPEVTCVVL | Exemplary variant canine |
| | DLGREDPEVQISWFVDGKEVHTAKTQSREQQFNGT | IgG-A Fc |
| | YRVVSVLPIEHQDWLTGKEFKCRVNHIDLPSPIER | Heterodimer knob |
| | TISKARGRAHKPSVYVLPPSPKELSSSDTVSIWCL | T(138)W |
| | IKDFYPPDIDVEWQSNGQQEPERKHRMTPPQLDED | |
| | GSYFLYSKLSVDKSRWQQGDPFTCAVMHETLQNHY | |
| | TDLSLSHSPGK | |
| 55 | PAPEMLGGPSVFIFPPKPKDTLLIARTPEVTCVVV | Exemplary variant canine |
| | DLDPEDPEVQISWFVDGKQMQTAKTQPREEQENGT | IgG-B Fc |
| | YRVVSVLPIGHQDWLKGKQFTCKVNNKALPSPIER | Heterodimer knob |
| | TISKARGQAHQPSVYVLPPSREELSKNTVSLWCLI | T(137)W |
| | KDFFPPDIDVEWQSNGQQEPESKYRTTPPQLDEDG | |
| | SYFLYSKLSVDKSRWQRGDTFICAVMHEALHNHYT | |
| | QESLSHSPGK | |
| 56 | PGCGLLGGPSVFIFPPKPKDILVTARTPTVTCVVV | Exemplary variant canine |
| | DLDPENPEVQISWFVDSKQVQTANTQPREEQSNGT | IgG-C Fc |
| | YRVVSVLPIGHQDWLSGKQFKCKVNNKALPSPIEE | Heterodimer knob |
| | IISKTPGQAHQPNVYVLPPSRDEMSKNTVTLWCLV | T(137)W |
| | KDFFPPEIDVEWQSNGQQEPESKYRMTPPQLDEDG | |
| | SYFLYSKLSVDKSRWQRGDTFICAVMHEALHNHYT | |
| | QISLSHSPGK | |
| 57 | PVPESLGGPSVFIFPPKPKDILRITRTPEITCVVL | Exemplary variant canine |
| | DLGREDPEVQISWFVDGKEVHTAKTQPREQQFNST | IgG-D Fc |
| | YRVVSVLPIEHQDWLTGKEFKCRVNHIGLPSPIER | Heterodimer knob |
| | TISKARGQAHQPSVYVLPPSPKELSSSDTVTLWCL | T(138)W |
| | IKDFFPPEIDVEWQSNGQPEPESKYHTTAPQLDED | |
| | GSYFLYSKLSVDKSRWQQGDTFTCAVMHEALQNHY | |
| | TDLSLSHSPGK | |
| 58 | PVPEPLGGPSVLIFPPKPKDILRITRTPEVTCVVL | Exemplary variant canine |
| | DLGREDPEVQISWFVDGKEVHTAKTQSREQQFNGT | IgG-A Fc |
| | YRVVSVLPIEHQDWLTGKEFKCRVNHIDLPSPIER | Heterodimer hole |
| | TISKARGRAHKPSVYVLPPSPKELSSSDTVSISCA | T(138)S |
| | IKDFYPPDIDVEWQSNGQQEPERKHRMTPPQLDED | L(140)A |
| | GSYFLYSKLSVDKSRWQQGDPFTCAVMHETLQNHY | |
| | TDLSLSHSPGK | |
| 59 | PAPEMLGGPSVFIFPPKPKDTLLIARTPEVTCVVV | Exemplary variant canine |
| | DLDPEDPEVQISWFVDGKQMQTAKTQPREEQFNGT | IgG-B Fc |
| | YRVVSVLPIGHQDWLKGKQFTCKVNNKALPSPIER | Heterodimer hole |
| | TISKARGQAHQPSVYVLPPSREELSKNTVSLSCAI | T(137)S |
| | KDFFPPDIDVEWQSNGQQEPESKYRTTPPQLDEDG | L(139)A |
| | SYFLYSKLSVDKSRWQRGDTFICAVMHEALHNHYT | |
| | QESLSHSPGK | |
| 60 | PGCGLLGGPSVFIFPPKPKDILVTARTPTVTCVVV | Exemplary variant canine |
| | DLDPENPEVQISWFVDSKQVQTANTQPREEQSNGT | IgG-C Fc |
| | YRVVSVLPIGHQDWLSGKQFKCKVNNKALPSPIEE | Heterodimer hole |
| | IISKTPGQAHQPNVYVLPPSRDEMSKNTVTLSCAV | T(137)S |
| | KDFFPPEIDVEWQSNGQQEPESKYRMTPPQLDEDG | L(139)A |
| | SYFLYSKLSVDKSRWQRGDTFICAVMHEALHNHYT | |
| | QISLSHSPGK | |
| 61 | PVPESLGGPSVFIFPPKPKDILRITRTPEITCVVL | Exemplary variant canine |
| | DLGREDPEVQISWFVDGKEVHTAKTQPREQQENST | IgG-D Fc |
| | YRVVSVLPIEHQDWLTGKEFKCRVNHIGLPSPIER | Heterodimer hole |
| | TISKARGQAHQPSVYVLPPSPKELSSSDTVTLSCA | T(138)S |
| | IKDFFPPEIDVEWQSNGQPEPESKYHTTAPQLDED | L(140)A |
| | GSYFLYSKLSVDKSRWQQGDTFTCAVMHEALQNHY | |
| | TDLSLSHSPGK | |
| 62 | PVPEPLGGPSVLIFPPKPKDILRITRTPEVTCVVL | Exemplary variant canine |
| | DLGREDPEVQISWFVDGKEVHTAKTQSREQQENGT | IgG-A Fc |
| | YRVVSVLPIEHQDWLTGKEFKCRVNHIDLPSPIER | Heterodimer hole |
| | TISKARGRAHKPSVYVLPPSPKELSSSDTVSISCA | T(138)S |
| | IKDFYPPDIDVEWQSNGQQEPERKHRMTPPQLDED | L(140)A |
| | GSYFLTSKLSVDKSRWQQGDPFTCAVMHETLQNHY | Y(181)T |
| | TDLSLSHSPGK | |
| 63 | PAPEMLGGPSVFIFPPKPKDTLLIARTPEVTCVVV | Exemplary variant canine |
| | DLDPEDPEVQISWFVDGKQMQTAKTQPREEQFNGT | IgG-B Fc |
| | YRVVSVLPIGHQDWLKGKQFTCKVNNKALPSPIER | Heterodimer hole |
| | TISKARGQAHQPSVYVLPPSREELSKNTVSLSCAI | T(137)S |
| | KDFFPPDIDVEWQSNGQQEPESKYRTTPPQLDEDG | L(139)A |
| | SYFLTSKLSVDKSRWQRGDTFICAVMHEALHNHYT | Y(180)T |
| | QESLSHSPGK | |
| 64 | PGCGLLGGPSVFIFPPKPKDILVTARTPTVTCVVV | Exemplary variant canine |
| | DLDPENPEVQISWFVDSKQVQTANTQPREEQSNGT | IgG-C Fc |
| | YRVVSVLPIGHQDWLSGKQFKCKVNNKALPSPIEE | Heterodimer hole |
| | IISKTPGQAHQPNVYVLPPSRDEMSKNTVTLSCAV | T(137)S |
| | KDFFPPEIDVEWQSNGQQEPESKYRMTPPQLDEDG | L(139)A |
| | SYFLTSKLSVDKSRWQRGDTFICAVMHEALHNHYT | Y(180)T |
| | QISLSHSPGK | |
| 65 | PVPESLGGPSVFIFPPKPKDILRITRTPEITCVVL | Exemplary variant canine |
| | DLGREDPEVQISWFVDGKEVHTAKTQPREQQFNST | IgG-D Fc |
| | YRVVSVLPIEHQDWLTGKEFKCRVNHIGLPSPIER | Heterodimer hole |
| | TISKARGQAHQPSVYVLPPSPKELSSSDTVTLSCA | T(138)S |
| | IKDFFPPEIDVEWQSNGQPEPESKYHTTAPQLDED | L(140)A |
| | GSYFLTSKLSVDKSRWQQGDTFTCAVMHEALQNHY | Y(181)T |
| | TDLSLSHSPGK | |
| 66 | RKTDHPPGPKPCDCPKCPPPEMLGGPSIFIFPPKP | Exemplary variant feline |
| | KDTLSISRTPEVTCLVVDLGPDDSDVQITWFVDNT | IgGla Fc |
| | QVYTAKTSPREEQFNSTYRVVSVLPILHQDWLKGK | Heterodimer knob |
| | EFKCKVNSKSLPSPIERTISKAKGQPHEPQVYVLP | T(154)W |
| | PAQEELSENKVSVWCLIKSFHPPDIAVEWEITGQP | |
| | EPENNYRTTPPQLDSDGTYFVYSKLSVDRSHWQRG | |
| | NTYTCSVSHEALHSHHTQKSLTQSPGK | |
| 67 | RKTDHPPGPKTGEGPKCPPPEMLGGPSIFIFPPKP | Exemplary variant feline |
| | KDTLSISRTPEVTCLVVDLGPDDSDVQITWFVDNT | IgGla Fc |
| | QVYTAKTSPREEQFNSTYRVVSVLPILHQDWLKGK | Heterodimer knob |
| | EFKCKVNSKSLPSPIERTISKAKGQPHEPQVYVLP | T(154)W |
| | PAQEELSENKVSVWCLIKSFHPPDIAVEWEITGQP | |
| | EPENNYRTTPPQLDSDGTYFVYSKLSVDRSHWQRG | |
| | NTYTCSVSHEALHSHHTQKSLTQSPGK | |
| 68 | RKTDHPPGPKPCDCPKCPPPEMLGGPSIFIFPPKP | Exemplary variant feline |
| | KDTLSISRTPEVTCLVVDLGPDDSDVQITWFVDNT | IgG1b Fc |
| | QVYTAKTSPREEQFNSTYRVVSVLPILHQDWLKGK | Heterodimer knob |
| | EFKCKVNSKSLPSPIERTISKDKGQPHEPQVYVLP | T(154)W |
| | PAQEELSENKVSVWCLIEGFYPSDIAVEWEITGQP | |
| | EPENNYRTTPPQLDSDGTYFLYSRLSVDRSRWQRG | |
| | NTYTCSVSHEALHSHHTQKSLTQSPGK | |
| 69 | RKTDHPPGPKTGEGPKCPPPEMLGGPSIFIFPPKP | Exemplary variant feline |
| | KDTLSISRTPEVTCLVVDLGPDDSDVQITWFVDNT | IgGlb Fc |
| | QVYTAKTSPREEQFNSTYRVVSVLPILHQDWLKGK | Heterodimer knob |
| | EFKCKVNSKSLPSPIERTISKDKGQPHEPQVYVLP | T(154)W |
| | PAQEELSENKVSVWCLIEGFYPSDIAVEWEITGQP | |
| | EPENNYRTTPPQLDSDGTYFLYSRLSVDRSRWQRG | |
| | NTYTCSVSHEALHSHHTQKSLTQSPGK | |
| 70 | PKTASTIESKTGEGPKCPVPEIPGAPSVFIFPPKP | Exemplary variant feline |
| | KDTLSISRTPEVTCLVVDLGPDDSNVQITWFVDNT | IgG2 Fc |
| | EMHTAKTRPREEQFNSTYRVVSVLPILHQDWLKGK | Heterodimer knob |
| | EFKCKVNSKSLPSAMERTISKAKGQPHEPQVYVLP | T(154)W |
| | PTQEELSENKVSVWCLIKGFHPPDIAVEWEITGQP | |
| | EPENNYQTTPPQLDSDGTYFLYSRLSVDRSHWQRG | |
| | NTYTCSVSHEALHSHHTQKSLTQSPGK | |
| 71 | RKTDHPPGPKPCDCPKCPPPEMLGGPSIFIFPPKP | Exemplary variant feline |
| | KDTLSISRTPEVTCLVVDLGPDDSDVQITWFVDNT | IgGla Fc |
| | QVYTAKTSPREEQFNSTYRVVSVLPILHQDWLKGK | Heterodimer hole |
| | EFKCKVNSKSLPSPIERTISKAKGQPHEPQVYVLP | T(154)S |
| | PAQEELSENKVSVSCAIKSFHPPDIAVEWEITGQP | L(156)A |
| | EPENNYRTTPPQLDSDGTYFVYSKLSVDRSHWQRG | |
| | NTYTCSVSHEALHSHHTQKSLTQSPGK | |
| 72 | RKTDHPPGPKTGEGPKCPPPEMLGGPSIFIFPPKP | Exemplary variant feline |
| | KDTLSISRTPEVTCLVVDLGPDDSDVQITWFVDNT | IgGla Fc |
| | QVYTAKTSPREEQFNSTYRVVSVLPILHQDWLKGK | Heterodimer hole |
| | EFKCKVNSKSLPSPIERTISKAKGQPHEPQVYVLP | T(154)S |
| | PAQEELSENKVSVSCAIKSFHPPDIAVEWEITGQP | L(156)A |
| | EPENNYRTTPPQLDSDGTYFVYSKLSVDRSHWQRG | |
| | NTYTCSVSHEALHSHHTQKSLTQSPGK | |
| 73 | RKTDHPPGPKPCDCPKCPPPEMLGGPSIFIFPPKP | Exemplary variant feline |
| | KDTLSISRTPEVTCLVVDLGPDDSDVQITWFVDNT | IgGlb Fc |
| | QVYTAKTSPREEQFNSTYRVVSVLPILHQDWLKGK | Heterodimer hole |
| | EFKCKVNSKSLPSPIERTISKDKGQPHEPQVYVLP | T(154)S |
| | PAQEELSENKVSVSCAIEGFYPSDIAVEWEITGQP | L(156)A |
| | EPENNYRTTPPQLDSDGTYFLYSRLSVDRSRWQRG | |
| | NTYTCSVSHEALHSHHTQKSLTQSPGK | |
| 74 | RKTDHPPGPKTGEGPKCPPPEMLGGPSIFIFPPKP | Exemplary variant feline |
| | KDTLSISRTPEVTCLVVDLGPDDSDVQITWFVDNT | IgGlb Fc |
| | QVYTAKTSPREEQFNSTYRVVSVLPILHQDWLKGK | Heterodimer hole |
| | EFKCKVNSKSLPSPIERTISKDKGQPHEPQVYVLP | T(154)S |
| | PAQEELSENKVSVSCAIEGFYPSDIAVEWEITGQP | L(156)A |
| | EPENNYRTTPPQLDSDGTYFLYSRLSVDRSRWQRG | |
| | NTYTCSVSHEALHSHHTQKSLTQSPGK | |
| 75 | PKTASTIESKTGEGPKCPVPEIPGAPSVFIFPPKP | Exemplary variant feline |
| | KDTLSISRTPEVTCLVVDLGPDDSNVQITWFVDNT | IgG2 Fc |
| | EMHTAKTRPREEQFNSTYRVVSVLPILHQDWLKGK | Heterodimer hole |
| | EFKCKVNSKSLPSAMERTISKAKGQPHEPQVYVLP | |
| | PTQEELSENKVSVSCAIKGFHPPDIAVEWEITGQP | |
| | EPENNYQTTPPQLDSDGTYFLYSRLSVDRSHWQRG | T(154)S |
| | NTYTCSVSHEALHSHHTQKSLTQSPGK | L(156)A |
| 76 | RKTDHPPGPKPCDCPKCPPPEMLGGPSIFIFPPKP | Exemplary variant feline |
| | KDTLSISRTPEVTCLVVDLGPDDSDVQITWFVDNT | IgGla Fc |
| | QVYTAKTSPREEQFNSTYRVVSVLPILHQDWLKGK | Heterodimer hole |
| | EFKCKVNSKSLPSPIERTISKAKGQPHEPQVYVLP | T(154)S |
| | PAQEELSENKVSVSCAIKSFHPPDIAVEWEITGQP | L(156)A |
| | EPENNYRTTPPQLDSDGTYFVTSKLSVDRSHWQRG | Y(197)T |
| | NTYTCSVSHEALHSHHTQKSLTQSPGK | |
| 77 | RKTDHPPGPKTGEGPKCPPPEMLGGPSIFIFPPKP | Exemplary variant feline |
| | KDTLSISRTPEVTCLVVDLGPDDSDVQITWFVDNT | IgGla Fc |
| | QVYTAKTSPREEQFNSTYRVVSVLPILHQDWLKGK | Heterodimer hole |
| | EFKCKVNSKSLPSPIERTISKAKGQPHEPQVYVLP | T(154)S |
| | PAQEELSENKVSVSCAIKSFHPPDIAVEWEITGQP | L(156)A |
| | EPENNYRTTPPQLDSDGTYFVTSKLSVDRSHWQRG | Y(197)T |
| | NTYTCSVSHEALHSHHTQKSLTQSPGK | |
| 78 | RKTDHPPGPKPCDCPKCPPPEMLGGPSIFIFPPKP | Exemplary variant feline |
| | KDTLSISRTPEVTCLVVDLGPDDSDVQITWFVDNT | IgG1b Fc |
| | QVYTAKTSPREEQFNSTYRVVSVLPILHQDWLKGK | Heterodimer hole |
| | EFKCKVNSKSLPSPIERTISKDKGQPHEPQVYVLP | T(154)S |
| | PAQEELSENKVSVSCAIEGFYPSDIAVEWEITGQP | L(156)A |
| | EPENNYRTTPPQLDSDGTYFLTSRLSVDRSRWQRG | Y(197)T |
| | NTYTCSVSHEALHSHHTQKSLTQSPGK | |
| 79 | RKTDHPPGPKTGEGPKCPPPEMLGGPSIFIFPPKP | Exemplary variant feline |
| | KDTLSISRTPEVTCLVVDLGPDDSDVQITWFVDNT | IgG1b Fc |
| | QVYTAKTSPREEQFNSTYRVVSVLPILHQDWLKGK | Heterodimer hole |
| | EFKCKVNSKSLPSPIERTISKDKGQPHEPQVYVLP | T(154)S |
| | PAQEELSENKVSVSCAIEGFYPSDIAVEWEITGQP | L(156)A |
| | EPENNYRTTPPQLDSDGTYFLTSRLSVDRSRWQRG | Y(197)T |
| | NTYTCSVSHEALHSHHTQKSLTQSPGK | |
| 80 | PKTASTIESKTGEGPKCPVPEIPGAPSVFIFPPKP | Exemplary variant feline |
| | KDTLSISRTPEVTCLVVDLGPDDSNVQITWFVDNT | IgG2 Fc |
| | EMHTAKTRPREEQFNSTYRVVSVLPILHQDWLKGK | Heterodimer hole |
| | EFKCKVNSKSLPSAMERTISKAKGQPHEPQVYVLP | T(154)S |
| | PTQEELSENKVSVSCAIKGFHPPDIAVEWEITGQP | L(156)A |
| | EPENNYQTTPPQLDSDGTYFLTSRLSVDRSHWQRG | Y(197)T |
| | NTYTCSVSHEALHSHHTQKSLTQSPGK | |
| 81 | GPSVFIFPPNPKDTLMITRTPEVTCVVVDVSQENP | Exemplary variant equine |
| | DVKFNWYMDGVEVRTATTRPKEEQFNSTYRVVSVL | IgG1 Fc |
| | RIQHQDWLSGKEFKCKVNNQALPQPIERTITKTKG | Heterodimer knob |
| | RSQEPQVYVLAPHPDELSKSKVSVWCLVKDFYPPE | T(130)W |
| | INIEWQSNGQPELETKYSTTQAQQDSDGSYFLYSK | |
| | LSVDRNRWQQGTTFTCGVMHEALHNHYTQKNVSKN | |
| | PGK | |
| 82 | GPSVFIFPPNPKDALMISRTPVVTCVVVNLSDQYP | Exemplary variant equine |
| | DVQFSWYVDNTEVHSAITKQREAQFNSTYRVVSVL | IgG2 Fc |
| | PIQHQDWLSGKEFKCSVTNVGVPQPISRAISRGKG | Heterodimer knob |
| | PSRVPQVYVLPPHPDELAKSKVSVWCLVKDFYPPD | T(130)W |
| | ISVEWQSNRWPELEGKYSTTPAQLDGDGSYFLYSK | |
| | LSLETSRWQQVESFTCAVMHEALHNHFTKTDISES | |
| | LGK | |
| 83 | GPSVFIFPPKPKDVLMITRMPEVTCLVVDVSHDSS | Exemplary variant equine |
| | DVLFTWYVDGTEVKTAKTMPNEEQNNSTYRVVSVL | IgG3 Fc |
| | RIQHQDWLNGKKFKCKVNNQALPAPVERTISKATG | Heterodimer knob |
| | QTRVPQVYVLAPHPDELSKNKVSVWCLVKDFYPPD | T(130)W |
| | ITVEWQSNEHPEPEGKYRTTEAQKDSDGSYFLYSK | |
| | LTVEKDRWQQGTTFTCVVMHEALHNHVMQKNISKN | |
| | PGK | |
| 84 | GPSVFIFPPKPKDVLMISRTPTVTCVVVDVGHDEP | Exemplary variant equine |
| | DVQFNWYVDGVETHTATTEPKQEQFNSTYRVVSVL | IgG4 Fc |
| | PIQHKDWLSGKEFKCKVNNKALPAPVERTISAPTG | Heterodimer knob |
| | QPREPQVYVLAPHRDELSKNKVSVWCLVKDFYPPD | T(130)W |
| | IDIEWKSNGQPEPETKYSTTPAQLDSDGSYFLYSK | |
| | LTVETNRWQQGTTFTCAVMHEALHNHYTEKSVSKS | |
| | PGK | |
| 85 | GPSVFIFPPKPKDVLMISRKPEVTCVVVDLGHDDP | Exemplary variant equine |
| | DVQFTWFVDGVETHTATTEPKEEQFNSTYRVVSVL | IgG5 Fc |
| | PIQHQDWLSGKEFKCSVTSKALPAPVERTISKAKG | Heterodimer knob |
| | QLRVPQVYVLAPHPDELAKNTVSVWCLVKDFYPPE | T(130)W |
| | IDVEWQSNEHPEPEGKYSTTPAQLNSDGSYFLYSK | |
| | LSVETSRWKQGESFTCGVMHEAVENHYTQKNVSHS | |
| | PGK | |
| 86 | RPSVFIFPPNPKDTLMISRTPEVTCVVVDVSQENP | Exemplary variant equine |
| | DVKFNWYVDGVEAHTATTKAKEKQDNSTYRVVSVL | IgG6 Fc |
| | PIQHQDWRRGKEFKCKVNNRALPAPVERTITKAKG | Heterodimer knob |
| | ELQDPKVYILAPHREEVTKNTVSVWCLVKDFYPPD | T(130)W |
| | INVEWQSNEEPEPEVKYSTTPAQLDGDGSYFLYSK | |
| | LTVETDRWEQGESFTCVVMHEAIRHTYRQKSITNF | |
| | PGK | |
| 87 | GPSVFIFPPKPKDVLMISRTPTVTCVVVDVGHDEP | Exemplary variant equine |
| | DVQFNWYVDGVETHTATTEPKQEQNNSTYRVVSIL | IgG7 Fc |
| | AIQHKDWLSGKEFKCKVNNQALPAPVQKTISKPTG | Heterodimer knob |
| | QPREPQVYVLAPHPDELSKNKVSVWCLVKDFYPPD | T(130)W |
| | IDIEWKSNGQPEPETKYSTTPAQLDGDGSYFLYSK | |
| | LTVETNRWQQGTTFTCAVMHEALHNHYTEKSVSKS | |
| | PGK | |
| 88 | GPSVFIFPPNPKDTLMITRTPEVTCVVVDVSQENP | Exemplary variant equine |
| | DVKFNWYMDGVEVRTATTRPKEEQFNSTYRVVSVL | IgG1 Fc |
| | RIQHQDWLSGKEFKCKVNNQALPQPIERTITKTKG | Heterodimer hole |
| | RSQEPQVYVLAPHPDELSKSKVSVSCAVKDFYPPE | T(130)S |
| | INIEWQSNGQPELETKYSTTQAQQDSDGSYFLYSK | L(132)A |
| | LSVDRNRWQQGTTFTCGVMHEALHNHYTQKNVSKN | |
| | PGK | |
| 89 | GPSVFIFPPNPKDALMISRTPVVTCVVVNLSDQYP | Exemplary variant equine |
| | DVQFSWYVDNTEVHSAITKQREAQFNSTYRVVSVL | IgG2 Fc |
| | PIQHQDWLSGKEFKCSVTNVGVPQPISRAISRGKG | Heterodimer hole |
| | PSRVPQVYVLPPHPDELAKSKVSVSCAVKDFYPPD | T(130)S |
| | ISVEWQSNRWPELEGKYSTTPAQLDGDGSYFLYSK | |
| | LSLETSRWQQVESFTCAVMHEALHNHFTKTDISES | L(132)A |
| | LGK | |
| 90 | GPSVFIFPPKPKDVLMITRMPEVTCLVVDVSHDSS | Exemplary variant equine |
| | DVLFTWYVDGTEVKTAKTMPNEEQNNSTYRVVSVL | IgG3 Fc |
| | RIQHQDWLNGKKFKCKVNNQALPAPVERTISKATG | Heterodimer hole |
| | QTRVPQVYVLAPHPDELSKNKVSVSCAVKDFYPPD | T(130)S |
| | ITVEWQSNEHPEPEGKYRTTEAQKDSDGSYFLYSK | L(132)A |
| | LTVEKDRWQQGTTFTCVVMHEALHNHVMQKNISKN | |
| | PGK | |
| 91 | GPSVFIFPPKPKDVLMISRTPTVTCVVVDVGHDEP | Exemplary variant equine |
| | DVQFNWYVDGVETHTATTEPKQEQFNSTYRVVSVL | IgG4 Fc |
| | PIQHKDWLSGKEFKCKVNNKALPAPVERTISAPTG | Heterodimer hole |
| | QPREPQVYVLAPHRDELSKNKVSVSCAVKDFYPPD | T(130)S |
| | IDIEWKSNGQPEPETKYSTTPAQLDSDGSYFLYSK | L(132)A |
| | LTVETNRWQQGTTFTCAVMHEALHNHYTEKSVSKS | |
| | PGK | |
| 92 | GPSVFIFPPKPKDVLMISRKPEVTCVVVDLGHDDP | Exemplary variant equine |
| | DVQFTWFVDGVETHTATTEPKEEQFNSTYRVVSVL | IgG5 Fc |
| | PIQHQDWLSGKEFKCSVTSKALPAPVERTISKAKG | Heterodimer hole |
| | QLRVPQVYVLAPHPDELAKNTVSVSCAVKDFYPPE | T(130)S |
| | IDVEWQSNEHPEPEGKYSTTPAQLNSDGSYFLYSK | L(132)A |
| | LSVETSRWKQGESFTCGVMHEAVENHYTQKNVSHS | |
| | PGK | |
| 93 | RPSVFIFPPNPKDTLMISRTPEVTCVVVDVSQEN | Exemplary variant equine |
| | PDVKENWYVDGVEAHTATTKAKEKQDNSTYRVVS | IgG6 Fc |
| | VLPIQHQDWRRGKEFKCKVNNRALPAPVERTITK | Heterodimer hole |
| | AKGELQDPKVYILAPHREEVTKNTVSVSCAVKDF | T(130)S |
| | YPPDINVEWQSNEEPEPEVKYSTTPAQLDGDGSY | L(132)A |
| | FLYSKLTVETDRWEQGESFTCVVMHEAIRHTYRQ | |
| | KSITNFPGK | |
| 94 | GPSVFIFPPKPKDVLMISRTPTVTCVVVDVGHDFP | Exemplary variant equine |
| | DVQFNWYVDGVETHTATTEPKQEQNNSTYRVVSIL | IgG7 Fc |
| | AIQHKDWLSGKEFKCKVNNQALPAPVQKTISKPTG | Heterodimer hole |
| | QPREPQVYVLAPHPDELSKNKVSVSCAVKDFYPPD | T(130)S |
| | IDIEWKSNGQPEPETKYSTTPAQLDGDGSYFLYSK | L(132)A |
| | LTVETNRWQQGTTFTCAVMHEALHNHYTEKSVSKS | |
| | PGK | |
| 95 | GPSVFIFPPNPKDTLMITRTPEVTCVVVDVSQENP | Exemplary variant equine |
| | DVKFNWYMDGVEVRTATTRPKEEQFNSTYRVVSVL | IgG1 Fc |
| | RIQHQDWLSGKEFKCKVNNQALPQPIERTITKTKG | Heterodimer hole |
| | RSQEPQVYVLAPHPDELSKSKVSVSCAVKDFYPPE | T(130)S |
| | INIEWQSNGQPELETKYSTTQAQQDSDGSYFLTSK | L(132)A |
| | LSVDRNRWQQGTTFTCGVMHEALHNHYTQKNVSKN | Y(173)T |
| | PGK | |
| 96 | GPSVFIFPPNPKDALMISRTPVVTCVVVNLSDQYP | Exemplary variant equine |
| | DVQFSWYVDNTEVHSAITKQREAQFNSTYRVVSVL | IgG2 Fc |
| | PIQHQDWLSGKEFKCSVTNVGVPQPISRAISRGKG | Heterodimer hole |
| | PSRVPQVYVLPPHPDELAKSKVSVSCAVKDFYPPD | T(130)S |
| | ISVEWQSNRWPELEGKYSTTPAQLDGDGSYFLTSK | |
| | LSLETSRWQQVESFTCAVMHEALHNHFTKTDISES | L(132)A |
| | LGK | Y(173)T |
| 97 | GPSVFIFPPKPKDVLMITRMPEVTCLVVDVSHDSS | Exemplary variant equine |
| | DVLFTWYVDGTEVKTAKTMPNEEQNNSTYRVVSVL | IgG3 Fc |
| | RIQHQDWLNGKKFKCKVNNQALPAPVERTISKATG | Heterodimer hole |
| | QTRVPQVYVLAPHPDELSKNKVSVSCAVKDFYPPD | T(130)S |
| | ITVEWQSNEHPEPEGKYRTTEAQKDSDGSYFLTSK | L(132)A |
| | LTVEKDRWQQGTTFTCVVMHEALHNHVMQKNISKN | Y(173)T |
| | PGK | |
| 98 | GPSVFIFPPKPKDVLMISRTPTVTCVVVDVGHDFP | Exemplary variant equine |
| | DVQFNWYVDGVETHTATTEPKQEQENSTYRVVSVL | IgG4 Fc |
| | PIQHKDWLSGKEFKCKVNNKALPAPVERTISAPTG | Heterodimer hole |
| | QPREPQVYVLAPHRDELSKNKVSVSCAVKDFYPPD | T(130)S |
| | IDIEWKSNGQPEPETKYSTTPAQLDSDGSYFLTSK | L(132)A |
| | LTVETNRWQQGTTFTCAVMHEALHNHYTEKSVSKS | Y(173)T |
| | PGK | |
| 99 | GPSVFIFPPKPKDVLMISRKPEVTCVVVDLGHDDP | Exemplary variant equine |
| | DVQFTWFVDGVETHTATTEPKEEQFNSTYRVVSVL | IgG5 Fc |
| | PIQHQDWLSGKEFKCSVTSKALPAPVERTISKAKG | Heterodimer hole |
| | QLRVPQVYVLAPHPDELAKNTVSVSCAVKDFYPPE | T(130)S |
| | IDVEWQSNEHPEPEGKYSTTPAQLNSDGSYFLTSK | L(132)A |
| | LSVETSRWKQGESFTCGVMHEAVENHYTQKNVSHS | Y(173)T |
| | PGK | |
| 100 | RPSVFIFPPNPKDTLMISRTPEVTCVVVDVSQENP | Exemplary variant equine |
| | DVKFNWYVDGVEAHTATTKAKEKQDNSTYRVVSVL | IgG6 Fc |
| | PIQHQDWRRGKEFKCKVNNRALPAPVERTITKAKG | Heterodimer hole |
| | ELQDPKVYILAPHREEVTKNTVSVSCAVKDFYPPD | T(130)S |
| | INVEWQSNEEPEPEVKYSTTPAQLDGDGSYFLTSK | L(132)A |
| | LTVETDRWEQGESFTCVVMHEAIRHTYRQKSITNF | Y(173)T |
| | PGK | |
| 101 | GPSVFIFPPKPKDVLMISRTPTVTCVVVDVGHDFP | Exemplary variant equine |
| | DVQFNWYVDGVETHTATTEPKQEQNNSTYRVVSIL | IgG7 Fc |
| | AIQHKDWLSGKEFKCKVNNQALPAPVQKTISKPTG | Heterodimer hole |
| | QPREPQVYVLAPHPDELSKNKVSVSCAVKDFYPPD | T(130)S |
| | IDIEWKSNGQPEPETKYSTTPAQLDGDGSYFLTSK | L(132)A |
| | LTVETNRWQQGTTFTCAVMHEALHNHYTEKSVSKS | Y(173)T |
| | PGK | |
| 102 | SGSVKVLHEPSCFSDYISTSVCQWKMDHPTNCSAE | Canine IL4R ECD canine |
| | LRLSYQLDEMGSENHTCVPENREDSVCVCSMPIDD | IgG-B Fc knob |
| | AVEADVYQLDLWAGQQLLWSGSFQPSKHVKPRTPG | |
| | NLTVHPNISHTWLLMWTNPYPTENHLHSELTYMVN | |
| | VSNDNDPEDFKVYNVTYMGPTLRLAASTLKSGASY | |
| | SARVRAWAQTYNSTWSDWSPSTTWLNYYEPGGGSG | |
| | GGGSGGGGSGGGGSGGGGSGPAPEMLGGPSVFIFP | |
| | PKPKDTLLIARTPEVTCVVVDLDPEDPEVQISWFV | |
| | DGKQMQTAKTQPREEQFNGTYRVVSVLPIGHQDWL | |
| | KGKQFTCKVNNKALPSPIERTISKARGQAHQPSVY | |
| | VLPPSREELSKNTVSLWCLIKDFFPPDIDVEWQSN | |
| | GQQEPESKYRTTPPQLDEDGSYFLYSKLSVDKSRW | |
| | QRGDTFICAVMHEALHNHYTQESLSHSPGK | |
| 103 | TETQPPVTNLSVSVENLCTVIWTWDPPEGASPNCT | Canine IL13R ECD canine |
| | LRYFSHEDNKQDKKIAPETHRSKEVPLNERICLQV | IgG-B Fc hole |
| | GSQCSTNESDNPSILVEKCTPPPEGDPESAVTELQ | |
| | CVWHNLSYMKCTWLPGRNTSPDTNYTLYYWHSSLG | |
| | KILQCEDIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETND | |
| | IFYVEEAKCQNSEFEGNLEGTICFMVPGVLPDTLN | |
| | TVRIRVRTNKLCYEDDKLWSNWSQAMSIGENTDPT | |
| | GGGSGGGGSGGGGSGGGGSGGGGSGPAPEMLGGPS | |
| | VFIFPPKPKDTLLIARTPEVTCVVVDLDPEDPEVQ | |
| | ISWFVDGKQMQTAKTQPREEQFNGTYRVVSVLPIG | |
| | HQDWLKGKQFTCKVNNKALPSPIERTISKARGQAH | |
| | QPSVYVLPPSREELSKNTVSLSCAIKDFFPPDIDV | |
| | EWQSNGQQEPESKYRTTPPQLDEDGSYFLTSKLSV | |
| | DKSRWQRGDTFICAVMHEALHNHYTQESLSHSPGK | |
| 104 | SGSVKVLHEPSCFSDYISTSVCQWKMDHPTNCSAE | Canine IL4R ECD canine |
| | LRLSYQLDFMGSENHTCVPENREDSVCVCSMPIDD | IgG-B Fc hole |
| | AVEADVYQLDLWAGQQLLWSGSFQPSKHVKPRTPG | |
| | NLTVHPNISHTWLLMWTNPYPTENHLHSELTYMVN | |
| | VSNDNDPEDFKVYNVTYMGPTLRLAASTLKSGASY | |
| | SARVRAWAQTYNSTWSDWSPSTTWLNYYEPGGGSG | |
| | GGGSGGGGSGGGGSGGGGSGPAPEMLGGPSVFIFP | |
| | PKPKDTLLIARTPEVTCVVVDLDPEDPEVQISWFV | |
| | DGKQMQTAKTQPREEQFNGTYRVVSVLPIGHQDWL | |
| | KGKQFTCKVNNKALPSPIERTISKARGQAHQPSVY | |
| | VLPPSREELSKNTVSLSCAIKDFFPPDIDVEWQSN | |
| | GQQEPESKYRTTPPQLDEDGSYFLTSKLSVDKSRW | |
| | QRGDTFICAVMHEALHNHYTQESLSHSPGK | |
| 105 | TETQPPVTNLSVSVENLCTVIWTWDPPEGASPNCT | Canine IL 13R ECD canine |
| | LRYFSHFDNKQDKKIAPETHRSKEVPLNERICLQV | IgG-B Fc knob |
| | GSQCSTNESDNPSILVEKCTPPPEGDPESAVTELQ | |
| | CVWHNLSYMKCTWLPGRNTSPDTNYTLYYWHSSLG | |
| | KILQCEDIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETND | |
| | IFYVEEAKCQNSEFEGNLEGTICFMVPGVLPDTLN | |
| | TVRIRVRTNKLCYEDDKLWSNWSQAMSIGENTDPT | |
| | GGGSGGGGSGGGGSGGGGSGGGGSGPAPEMLGGPS | |
| | VFIFPPKPKDTLLIARTPEVTCVVVDLDPEDPEVQ | |
| | ISWFVDGKQMQTAKTQPREEQFNGTYRVVSVLPIG | |
| | HQDWLKGKQFTCKVNNKALPSPIERTISKARGQAH | |
| | QPSVYVLPPSREELSKNTVSLWCLIKDFFPPDIDV | |
| | EWQSNGQQEPESKYRTTPPQLDEDGSYFLYSKLSV | |
| | DKSRWQRGDTFICAVMHEALHNHYTQESLSHSPGK | |
| 106 | SGSVKVLRAPTCFSDYFSTSVCQWNMDAPTNCSAE | Feline IL4R ECD feline IgG- |
| | LRLSYQLNFMGSENRTCVPENGEGAACACSMLMDD | 2 Fc knob |
| | FVEADVYQLHLWAGTQLLWSGSFKPSSHVKPRAPG | |
| | NLTVHPNVSHTWLLRWSNPYPPENHLHAELTYMVN | |
| | ISSEDDPTDVSVCASGFLCHLLGLRRVETGAPGAR | |
| | LPPWLCAPRPRRVPGSQCAVISCCRWVLIALTSRG | |
| | GRWRLTPGLRSQTRYVSVAEGLFGATPRVLCPGTQ | |
| | AGLASAAREQMSPDPSAFHSIDYEPGGGSGGGGSG | |
| | GGGSGGGGSGGGGSGPKTASTIESKTGEGPKCPVP | |
| | EIPGAPSVFIFPPKPKDTLSISRTPEVTCLVVDLG | |
| | PDDSNVQITWFVDNTEMHTAKTRPREEQENSTYRV | |
| | VSVLPILHQDWLKGKEFKCKVNSKSLPSAMERTIS | |
| | KAKGQPHEPQVYVLPPTQEELSENKVSVWCLIKGE | |
| | HPPDIAVEWEITGQPEPENNYQTTPPQLDSDGTYF | |
| | LYSRLSVDRSHWQRGNTYTCSVSHEALHSHHTQKS | |
| | LTQSPGK | |
| 107 | SQTQPPVTNLSVSVENLCTVIWTWDPPEGASPNCT | Feline IL13R ECD feline |
| | LRYFSHFDNKQDKKIAPETHRSKEVPLNERICLQV | IgG-2 Fc hole |
| | GSQCSTNESDNPSILVEKCTPPPEGDPESAVTELQ | |
| | CVWHNLSYMKCTWLPGRNTSPDTNYTLYYWHSSLG | |
| | KILQCENIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETHD | |
| | IFYVEEAKCQNSEFEGNLEGTICFMVPGILPDTLN | |
| | TVRIRVRTNKLCYEDDRLWSNWSQAMSIGENTDPT | |
| | GGGSGGGGSGGGGSGGGGSGGGGSGPKTASTIESK | |
| | TGEGPKCPVPEIPGAPSVFIFPPKPKDTLSISRTP | |
| | EVTCLVVDLGPDDSNVQITWFVDNTEMHTAKTRPR | |
| | EEQFNSTYRVVSVLPILHQDWLKGKEFKCKVNSKS | |
| | LPSAMERTISKAKGQPHEPQVYVLPPTQEELSENK | |
| | VSVSCAIKGFHPPDIAVEWEITGQPEPENNYQTTP | |
| | PQLDSDGTYFLTSRLSVDRSHWQRGNTYTCSVSHE | |
| | ALHSHHTQKSLTQSPGK | |
| 108 | SGSVKVLRAPTCFSDYFSTSVCQWNMDAPTNCSAE | Feline IL4R ECD feline IgG- |
| | LRLSYQLNFMGSENRTCVPENGEGAACACSMLMDD | 2 Fc hole |
| | FVEADVYQLHLWAGTQLLWSGSFKPSSHVKPRAPG | |
| | NLTVHPNVSHTWLLRWSNPYPPENHLHAELTYMVN | |
| | ISSEDDPTDVSVCASGFLCHLLGLRRVETGAPGAR | |
| | LPPWLCAPRPRRVPGSQCAVISCCRWVLIALTSRG | |
| | GRWRLTPGLRSQTRYVSVAEGLFGATPRVLCPGTQ | |
| | AGLASAAREQMSPDPSAFHSIDYEPGGGSGGGGSG | |
| | GGGSGGGGSGGGGSGPKTASTIESKTGEGPKCPVP | |
| | EIPGAPSVFIFPPKPKDTLSISRTPEVTCLVVDLG | |
| | PDDSNVQITWFVDNTEMHTAKTRPREEQFNSTYRV | |
| | VSVLPILHQDWLKGKEFKCKVNSKSLPSAMERTIS | |
| | KAKGQPHEPQVYVLPPTQEELSENKVSVSCAIKGF | |
| | HPPDIAVEWEITGQPEPENNYQTTPPQLDSDGTYF | |
| | LTSRLSVDRSHWQRGNTYTCSVSHEALHSHHTQKS | |
| | LTQSPGK | |
| 109 | SQTQPPVTNLSVSVENLCTVIWTWDPPEGASPNCT | Feline IL 13R ECD feline |
| | LRYFSHFDNKQDKKIAPETHRSKEVPLNERICLQV | IgG-2 Fc knob |
| | GSQCSTNESDNPSILVEKCTPPPEGDPESAVTELQ | |
| | CVWHNLSYMKCTWLPGRNTSPDTNYTLYYWHSSLG | |
| | KILQCENIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETHD | |
| | IFYVEEAKCQNSEFEGNLEGTICFMVPGILPDTLN | |
| | TVRIRVRTNKLCYEDDRLWSNWSQAMSIGENTDPT | |
| | GGGSGGGGSGGGGSGGGGSGGGGSGPKTASTIESK | |
| | TGEGPKCPVPEIPGAPSVFIFPPKPKDTLSISRTP | |
| | EVTCLVVDLGPDDSNVQITWFVDNTEMHTAKTRPR | |
| | EEQFNSTYRVVSVLPILHQDWLKGKEFKCKVNSKS | |
| | LPSAMERTISKAKGQPHEPQVYVLPPTQEELSENK | |
| | VSVWCLIKGFHPPDIAVEWEITGQPEPENNYQTTP | |
| | PQLDSDGTYFLYSRLSVDRSHWQRGNTYTCSVSHE | |
| | ALHSHHTQKSLTQSPGK | |
| 110 | SGSVKVLHLTACESDYISASTCEWKMDRPTNCSAQ | Equine IL4R ECD equine |
| | LRLSYQLNDEFSDNLTCIPENREDEVCVCRMLMDN | IgG-2 Fc knob |
| | IVSEDVYELDLWAGNQLLWNSSFKPSRHVKPRAPQ | |
| | NLTVHAISHTWLLTWSNPYPLKNHLWSELTYLVNI | |
| | SKEDDPTDFKIYNVTYMDPTLRVTASTLKSRATYS | |
| | ARVKARAQNYNSTWSEWSPSTTWHNYYEQPGGGSG | |
| | GGGSGGGGSGGGGSGGGGSGGPSVFIFPPNPKDAL | |
| | MISRTPVVTCVVVNLSDQYPDVQFSWYVDNTEVHS | |
| | AITKQREAQFNSTYRVVSVLPIQHQDWLSGKEFKC | |
| | SVTNVGVPQPISRAISRGKGPSRVPQVYVLPPHPD | |
| | ELAKSKVSVWCLVKDFYPPDISVEWQSNRWPELEG | |
| | KYSTTPAQLDGDGSYFLYSKLSLETSRWQQVESFT | |
| | CAVMHEALHNHFTKTDISESLGK | |
| 111 | TESQPPVTNLSVSVENLCTVIWTWNPPEGVSPNCS | Equine IL13R ECD equine |
| | LWYFSHEGNKQDKKIAPETHRSKEVPLNERICLQV | IgG-2 Fc hole |
| | GSQCSTNESDNPSILVEKCISPPEGDPESAVTELQ | |
| | CVWHNLSYMKCTWLPGKNASPDTNYTLYYWHSSLG | |
| | KILQCEDIYREGQHIGCSFALTEVKDSIFEQHSVQ | |
| | IMVKDNAGKIRPFFNIVPLTSHVKPDPPHIKKLFF | |
| | QNGDLYVQWKNPQNFYSRCLSYQVEVNNSQTETRD | |
| | IFSVEEAKCQNPEFEGDLEGTICFMVPGVLPDTVN | |
| | TVRIRVKTNKLCYEDDKLWSNWSQAMSIGKKADPT | |
| | GGGSGGGGSGGGGSGGGGSGGGGSGGPSVFIFPPN | |
| | PKDALMISRTPVVTCVVVNLSDQYPDVQFSWYVDN | |
| | TEVHSAITKQREAQFNSTYRVVSVLPIQHQDWLSG | |
| | KEFKCSVTNVGVPQPISRAISRGKGPSRVPQVYVL | |
| | PPHPDELAKSKVSVSCAVKDFYPPDISVEWQSNRW | |
| | PELEGKYSTTPAQLDGDGSYFLTSKLSLETSRWQQ | |
| | VESFTCAVMHEALHNHFTKTDISESLGK | |
| 112 | SGSVKVLHLTACFSDYISASTCEWKMDRPTNCSAQ | Equine IL4R ECD equine |
| | LRLSYQLNDEFSDNLTCIPENREDEVCVCRMLMDN | IgG-2 Fc hole |
| | IVSEDVYELDLWAGNQLLWNSSFKPSRHVKPRAPQ | |
| | NLTVHAISHTWLLTWSNPYPLKNHLWSELTYLVNI | |
| | SKEDDPTDFKIYNVTYMDPTLRVTASTLKSRATYS | |
| | ARVKARAQNYNSTWSEWSPSTTWHNYYEQPGGGSG | |
| | GGGSGGGGSGGGGSGGGGSGGPSVFIFPPNPKDAL | |
| | MISRTPVVTCVVVNLSDQYPDVQFSWYVDNTEVHS | |
| | AITKQREAQFNSTYRVVSVLPIQHQDWLSGKEFKC | |
| | SVTNVGVPQPISRAISRGKGPSRVPQVYVLPPHPD | |
| | ELAKSKVSVSCAVKDFYPPDISVEWQSNRWPELEG | |
| | KYSTTPAQLDGDGSYFLTSKLSLETSRWQQVESFT | |
| | CAVMHEALHNHETKTDISESLGK | |
| 113 | TESQPPVTNLSVSVENLCTVIWTWNPPEGVSPNCS | Equine IL13R ECD equine |
| | LWYFSHFGNKQDKKIAPETHRSKEVPLNERICLQV | IgG-2 Fc knob |
| | GSQCSTNESDNPSILVEKCISPPEGDPESAVTELQ | |
| | CVWHNLSYMKCTWLPGKNASPDTNYTLYYWHSSLG | |
| | KILQCEDIYREGQHIGCSFALTEVKDSIFEQHSVQ | |
| | IMVKDNAGKIRPFFNIVPLTSHVKPDPPHIKKLFF | |
| | QNGDLYVQWKNPQNFYSRCLSYQVEVNNSQTETRD | |
| | IFSVEEAKCQNPEFEGDLEGTICFMVPGVLPDTVN | |
| | TVRIRVKTNKLCYEDDKLWSNWSQAMSIGKKADPT | |
| | GGGSGGGGSGGGGSGGGGSGGGGSGGPSVFIFPPN | |
| | PKDALMISRTPVVTCVVVNLSDQYPDVQFSWYVDN | |
| | TEVHSAITKQREAQFNSTYRVVSVLPIQHQDWLSG | |
| | KEFKCSVTNVGVPQPISRAISRGKGPSRVPQVYVL | |
| | PPHPDELAKSKVSVWCLVKDFYPPDISVEWQSNRW | |
| | PELEGKYSTTPAQLDGDGSYFLYSKLSLETSRWQQ | |
| | VESFTCAVMHEALHNHETKTDISESLGK | |
| 114 | MGVPRPRSWGLGFLLFLLPTLRAADSHLSLLYHLT | Exemplary canine FcRn with |
| | AVSAPPPGTPAFWASGWLGPQQYLSYNNLRAQAEP | poly-His |
| | YGAWVWENQVSWYWEKETTDLRTKEGLFLEALKAL | |
| | GDGGPYTLQGLLGCELGPDNTSVPVAKFALNGEDE | |
| | MTFDPKLGTWNGDWPETETVSKRWMQQAGAVSKER | |
| | TFLLYSCPQRLLGHLERGRGNLEWKEPPSMRLKAR | |
| | PGSPGFSVLTCSAFSFYPPELQLRELRNGLAAGSG | |
| | EGDFGPNGDGSFHAWSSLTVKSGDEHHYRCLVQHA | |
| | GLPQPLTVELESPAKSSGSHHHHHH | |
| 115 | MAPRPALATAGFLALLLILLAACRLDAVQHPPKIQ | Exemplary canine B2M |
| | VYSRHPAENGKPNFLNCYVSGFHPPEIEIDLLKNG | |
| | KEMKAEQTDLSFSKDWTFYLLVHTEFTPNEQDEFS | |
| | CRVKHVTLSEPQIVKWDRDN | |
| 116 | PAPEMLGGPSVFIFPPKPKDTLFIARTPEVTCVVV | Exemplary variant canine |
| | DLDPEDPEVQISWFVDGKQMQTAKTQPREEQENGT | IgG-B Fc |
| | YRVVSVLPIGHQDWLKGKQFTCKVNNKALPSPIER | Protein A+ |
| | TISKARGQAHQPSVYVLPPSREELSKNTVSLTCLI | C1q+ |
| | KDFFPPDIDVEWQSNGQQEPESKYRTTPPQLDEDG | CD16+ |
| | SYFLYSKLSVDKSRWQRGDTFICAVMHEALHNHYT | L(23)F (F00) |
| | QESLSHSPGK | |
| 117 | PAPEMLGGPSVFIFPPKPKDTLYIARTPEVTCVVV | Exemplary variant canine |
| | DLDPEDPEVQISWFVDGKQMQTAKTQPREEQENGT | IgG-B Fc |
| | YRVVSVLPIGHQDWLKGKQFTCKVNNKALPSPIER | Protein A+ |
| | TISKARGQAHQPSVYVLPPSREELSKNTVSLTCLI | C1q+ |
| | KDFFPPDIDVEWQSNGQQEPESKYRTTPPQLDEDG | CD16+ |
| | SYFLYSKLSVDKSRWQRGDTFICAVMHEALHNHYT | L(23)Y (Y00) |
| | QESLSHSPGK | |
| 118 | PVPEPLGGPSVLIFPPKPKDTLFIARTPEVTCVVL | Exemplary variant canine |
| | DLGREDPEVQISWFVDGKEVHTAKTQSREQQFNGT | IgG-A Fc (F00; Protein A+; |
| | YRVVSVLPIGHQDWLTGKEFKCRVNHIDLPSPIER | C1q−; CD16−) |
| | TISKARGRAHKPSVYVLPPSPKELSSSDTVSITCL | I(21)T; R(23)F; T(25)A; |
| | IKDFYPPDIDVEWQSNGQQEPERKHRMTPPQLDED | E(80)G; T(205)A; Q(207)H |
| | GSYFLYSKLSVDKSRWQQGDPFTCAVMHEALHNHY | |
| | TDLSLSHSPGK | |
| 119 | PAPEMLGGPSVLIFPPKPKDTLLIARTPEVTCVVV | Exemplary variant canine |
| | DLDPEDPEVQISWFVDGKEVHTAKTQSREEQENGT | IgG-A Fc (Protein A+; |
| | YRVVSVLPIGHQDWLTGKEFKCKVNNKALPSPIER | C1q+; CD16 +) |
| | TISKARGRAHKPSVYVLPPSPKELSSSDTVSITCL | V2A; P5M; I21T; R23L; |
| | IKDFYPPDIDVEWQSNGQQEPERKHRMTPPQLDED | T25A; L35V; G38D; R39P; |
| | GSYFLYSKLSVDKSRWQQGDPFTCAVMHEALHNHY | Q65E; E80G; R93K; H96N; |
| | TDLSLSHSPGK | I97K; D98A; T205A; Q207H |
| 120 | CPVPESLGGPSVFIFPPKPKDTLFIARTPEITCVV | Exemplary variant canine |
| | LDLGREDPEVQISWFVDGKEVHTAKTQPREQQENS | IgG-D Fc (F00; Protein A+; |
| | TYRVVSVLPIGHQDWLTGKEFKCRVNHIGLPSPIE | C1q−; CD16−) |
| | RTISKARGQAHQPSVYVLPPSPKELSSSDTVTLTC | I(21)T; R(23)F; T(25)A; |
| | LIKDFFPPEIDVEWQSNGQPEPESKYHTTAPQLDE | E(80)G; Q(205)A; Q(207)H |
| | DGSYFLYSKLSVDKSRWQQGDTFTCAVMHEALHNH | |
| | YTDLSLSHSPGK | |
| 121 | CPAPEMLGGPSVFIFPPKPKDTLLIARTPEITCVV | Exemplary variant canine |
| | VDLDPEDPEVQISWFVDGKEVHTAKTQPREEQENS | IgG-D Fc (Protein A+; |
| | TYRVVSVLPIGHQDWLTGKEFKCKVNNKALPSPIE | C1q+; CD16 +) |
| | RTISKARGQAHQPSVYVLPPSPKELSSSDTVTLTC | V2A; S5M; I21T; R23L; |
| | LIKDFFPPEIDVEWQSNGQPEPESKYHTTAPQLDE | T25A; L35V; G38D; R39P; |
| | DGSYFLYSKLSVDKSRWQQGDTFTCAVMHEALHNH | Q65E; E80G; R93K; H96N; |
| | YTDLSLSHSPGK | I97K; G98A; Q207H |
| 122 | PAPEMLGGPSVFIFPPKPKDTLLIARTPEVTCVVV | Exemplary variant canine |
| | DLDPEDPEVQISWFVDGKQMQTAKTQPREEQENGT | IgG-B Fc (0Y0) |
| | YRVVSVLPIGHYDWLKGKQFTCKVNNKALPSPIER | Protein A + |
| | TISKARGQAHQPSVYVLPPSREELSKNTVSLTCLI | C1q+ |
| | KDFFPPDIDVEWQSNGQQEPESKYRTTPPQLDEDG | CD16+ |
| | SYFLYSKLSVDKSRWQRGDTFICAVMHEALHNHYT | Q(82)Y (0Y0) |
| | QESLSHSPGK | |
| 123 | PAPEMLGGPSVFIFPPKPKDTLLIARTPEVTCVVV | Exemplary variant canine |
| | DLDPEDPEVQISWFVDGKQMQTAKTQPREEQENGT | IgG-B Fc (0YH) |
| | YRVVSVLPIGHYDWLKGKQFTCKVNNKALPSPIER | Gln82Tyr |
| | TISKARGQAHQPSVYVLPPSREELSKNTVSLTCLI | Asn207His |
| | KDFFPPDIDVEWQSNGQQEPESKYRTTPPQLDEDG | |
| | SYFLYSKLSVDKSRWQRGDTFICAVMHEALHHHYT | |
| | QESLSHSPGK | |
| 124 | PAPEMLGGPSVFIFPPKPKDTLLIARTPEVTCVVV | Exemplary variant canine |
| | DLDPEDPEVQISWFVDGKQMQTAKTQPREEQENGT | IgG-B Fc (0YY) |
| | YRVVSVLPIGHYDWLKGKQFTCKVNNKALPSPIER | Gln82Tyr |
| | TISKARGQAHQPSVYVLPPSREELSKNTVSLTCLI | Asn207Tyr |
| | KDFFPPDIDVEWQSNGQQEPESKYRTTPPQLDEDG | |
| | SYFLYSKLSVDKSRWQRGDTFICAVMHEALHYHYT | |
| | QESLSHSPGK | |
| 125 | PAPEMLGGPSVFIFPPKPKDTLLIARTPEVTCVVV | Exemplary variant canine |
| | DLDPEDPEVQISWFVDGKQMQTAKTQPREEQENGT | IgG-B Fc (00Y) |
| | YRVVSVLPIGHQDWLKGKQFTCKVNNKALPSPIER | Asn207Tyr |
| | TISKARGQAHQPSVYVLPPSREELSKNTVSLTCLI | |
| | KDFFPPDIDVEWQSNGQQEPESKYRTTPPQLDEDG | |
| | SYFLYSKLSVDKSRWQRGDTFICAVMHEALHYHYT | |
| | QESLSHSPGK | |
| 126 | PAPEMLGGPSVFIFPPKPKDTLYITREPEVTCVVV | Exemplary variant canine |
| | DLDPEDPEVQISWFVDGKQMQTAKTQPREEQENGT | IgG-B Fc (YTE) |
| | YRVVSVLPIGHQDWLKGKQFTCKVNNKALPSPIER | Leu23Tyr |
| | TISKARGQAHQPSVYVLPPSREELSKNTVSLTCLI | Ala25Thr |
| | KDFFPPDIDVEWQSNGQQEPESKYRTTPPQLDEDG | Thr27Glu |
| | SYFLYSKLSVDKSRWQRGDTFICAVMHEALHNHYT | |
| | QESLSHSPGK | |
| 127 | PAPEMLGGPSVFIFPPKPKDTLFIARTPEVTCVVV | Exemplary variant canine |
| | DLDPEDPEVQISWFVDGKQMQTAKTQPREEQENGT | IgG-B Fc |
| | YRVVSVLPIGHQDWLKGKQFTCRVNNIGLPSPIER | Protein A + |
| | TISKARGQAHQPSVYVLPPSREELSKNTVSLTCLI | C1q− |
| | KDFFPPDIDVEWQSNGQQEPESKYRTTPPQLDEDG | CD16− |
| | SYFLYSKLSVDKSRWQRGDTFICAVMHEALHNHYT | K(93)R |
| | QESLSHSPGK | K(97)I |
| | | A(98)G |
| | | L(23)F (F00) |
| 128 | PAPEMLGGPSVFIFPPKPKDTLYIARTPEVTCVVV | Exemplary variant canine |
| | DLDPEDPEVQISWFVDGKQMQTAKTQPREEQFNGT | IgG-B Fc |
| | YRVVSVLPIGHQDWLKGKQFTCRVNNIGLPSPIER | Protein A + |
| | TISKARGQAHQPSVYVLPPSREELSKNTVSLTCLI | C1q− |
| | KDFFPPDIDVEWQSNGQQEPESKYRTTPPQLDEDG | CD16− |
| | SYFLYSKLSVDKSRWQRGDTFICAVMHEALHNHYT | K(93)R |
| | QESLSHSPGK | K(97)I |
| | | A(98)G |
| | | L(23)Y (Y00) |
| 129 | PAPEMLGGPSVFIFPPKPKDTLLIARTPEVTCVVV | Exemplary variant canine |
| | DLDPEDPEVQISWFVDGKQMQTAKTQPREEQFNGT | IgG-B Fc |
| | YRVVSVLPIGHYDWLKGKQFTCRVNNIGLPSPIER | Protein A + |
| | TISKARGQAHQPSVYVLPPSREELSKNTVSLTCLI | C1q− |
| | KDFFPPDIDVEWQSNGQQEPESKYRTTPPQLDEDG | CD16− |
| | SYFLYSKLSVDKSRWQRGDTFICAVMHEALHNHYT | K(93)R |
| | QESLSHSPGK | K(97)I |
| | | A(98)G |
| | | Q(82)Y (00) |
| 130 | SGSVKVLHEPSCFSDYISTSVCQWKMDHPTNCSAE | Exemplary canine |
| | LRLSYQLDFMGSENHTCVPENREDSVCVCSMPIDD | IL4RECD-IL13RECD-IgGB |
| | AVEADVYQLDLWAGQQLLWSGSFQPSKHVKPRTPG | Fc |
| | NLTVHPNISHTWLLMWTNPYPTENHLHSELTYMVN | (without signal sequence) |
| | VSNDNDPEDFKVYNVTYMGPTLRLAASTLKSGASY | (C may be substituted with: |
| | SARVRAWAQTYNSTWSDWSPSTTWLNYYEPGGGSG | A, V, S) |
| | TETQPPVTNLSVSVENLCTVIWTWDPPEGASPNCT | |
| | LRYFSHFDNKQDKKIAPETHRSKEVPLNERICLQV | |
| | GSQCSTNESDNPSILVEKCTPPPEGDPESAVTELQ | |
| | CVWHNLSYMKCTWLPGRNTSPDTNYTLYYWHSSLG | |
| | KILQCEDIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETND | |
| | IFYVEEAKCQNSEFEGNLEGTICFMVPGVLPDTLN | |
| | TVRIRVRTNKLCYEDDKLWSNWSQAMSIGENTDPT | |
| | PKRENGRVPRPPDCPKCPAPEMLGGPSVFIFPPKP | |
| | KDTLLIARTPEVTCVVVDLDPEDPEVQISWFVDGK | |
| | QMQTAKTQPREEQFNGTYRVVSVLPIGHQDWLKGK | |
| | QFTCKVNNKALPSPIERTISKARGQAHQPSVYVLP | |
| | PSREELSKNTVSLTCLIKDFFPPDIDVEWQSNGQQ | |
| | EPESKYRTTPPQLDEDGSYFLYSKLSVDKSRWQRG | |
| | DTFICAVMHEALHNHYTQESLSHSPGK | |
| 131 | SGSVKVLHEPSCFSDYISTSVCQWKMDHPTNCSAE | Exemplary canine |
| | LRLSYQLDEMGSENHTCVPENREDSVCVCSMPIDD | IL4RECD-IL13RECD-IgGB |
| | AVEADVYQLDLWAGQQLLWSGSFQPSKHVKPRTPG | Fc (without signal sequence) |
| | NLTVHPNISHTWLLMWTNPYPTENHLHSELTYMVN | |
| | VSNDNDPEDFKVYNVTYMGPTLRLAASTLKSGASY | |
| | SARVRAWAQTYNSTWSDWSPSTTWLNYYEPGGGSG | |
| | TETQPPVTNLSVSVENLATVIWTWDPPEGASPNCT | |
| | LRYFSHFDNKQDKKIAPETHRSKEVPLNERICLQV | |
| | GSQCSTNESDNPSILVEKCTPPPEGDPESAVTELQ | |
| | CVWHNLSYMKCTWLPGRNTSPDTNYTLYYWHSSLG | |
| | KILQCEDIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETND | |
| | IFYVEEAKCQNSEFEGNLEGTICFMVPGVLPDTLN | |
| | TVRIRVRTNKLCYEDDKLWSNWSQAMSIGENTDPT | |
| | PKRENGRVPRPPDCPKCPAPEMLGGPSVFIFPPKP | |
| | KDTLLIARTPEVTCVVVDLDPEDPEVQISWFVDGK | |
| | QMQTAKTQPREEQFNGTYRVVSVLPIGHQDWLKGK | |
| | QFTCKVNNKALPSPIERTISKARGQAHQPSVYVLP | |
| | PSREELSKNTVSLTCLIKDFFPPDIDVEWQSNGQQ | |
| | EPESKYRTTPPQLDEDGSYFLYSKLSVDKSRWQRG | |
| | DTFICAVMHEALHNHYTQESLSHSPGK | |
| 132 | SGSVKVLHEPSCFSDYISTSVCQWKMDHPTNCSAE | Exemplary canine |
| | LRLSYQLDEMGSENHTCVPENREDSVCVCSMPIDD | IL4RECD-IL13RECD-IgGB |
| | AVEADVYQLDLWAGQQLLWSGSFQPSKHVKPRTPG | Fc (without signal sequence) |
| | NLTVHPNISHTWLLMWTNPYPTENHLHSELTYMVN | |
| | VSNDNDPEDFKVYNVTYMGPTLRLAASTLKSGASY | |
| | SARVRAWAQTYNSTWSDWSPSTTWLNYYEPGGGSG | |
| | TETQPPVTNLSVSVENLVTVIWTWDPPEGASPNCT | |
| | LRYFSHFDNKQDKKIAPETHRSKEVPLNERICLQV | |
| | GSQCSTNESDNPSILVEKCTPPPEGDPESAVTELQ | |
| | CVWHNLSYMKCTWLPGRNTSPDTNYTLYYWHSSLG | |
| | KILQCEDIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETND | |
| | IFYVEEAKCQNSEFEGNLEGTICFMVPGVLPDTLN | |
| | TVRIRVRTNKLCYEDDKLWSNWSQAMSIGENTDPT | |
| | PKRENGRVPRPPDCPKCPAPEMLGGPSVFIFPPKP | |
| | KDTLLIARTPEVTCVVVDLDPEDPEVQISWFVDGK | |
| | QMQTAKTQPREEQFNGTYRVVSVLPIGHQDWLKGK | |
| | QFTCKVNNKALPSPIERTISKARGQAHQPSVYVLP | |
| | PSREELSKNTVSLTCLIKDFFPPDIDVEWQSNGQQ | |
| | EPESKYRTTPPQLDEDGSYFLYSKLSVDKSRWQRG | |
| | DTFICAVMHEALHNHYTQESLSHSPGK | |
| 133 | SGSVKVLHEPSCFSDYISTSVCQWKMDHPTNCSAE | Exemplary canine |
| | LRLSYQLDFMGSENHTCVPENREDSVCVCSMPIDD | IL4RECD-IL13RECD-IgGB |
| | AVEADVYQLDLWAGQQLLWSGSFQPSKHVKPRTPG | Fc (without signal sequence) |
| | NLTVHPNISHTWLLMWTNPYPTENHLHSELTYMVN | |
| | VSNDNDPEDFKVYNVTYMGPTLRLAASTLKSGASY | |
| | SARVRAWAQTYNSTWSDWSPSTTWLNYYEPGGGSG | |
| | TETQPPVTNLSVSVENLSTVIWTWDPPEGASPNCT | |
| | LRYFSHFDNKQDKKIAPETHRSKEVPLNERICLQV | |
| | GSQCSTNESDNPSILVEKCTPPPEGDPESAVTELQ | |
| | CVWHNLSYMKCTWLPGRNTSPDTNYTLYYWHSSLG | |
| | KILQCEDIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETND | |
| | IFYVEEAKCQNSEFEGNLEGTICFMVPGVLPDTLN | |
| | TVRIRVRTNKLCYEDDKLWSNWSQAMSIGENTDPT | |
| | PKRENGRVPRPPDCPKCPAPEMLGGPSVFIFPPKP | |
| | KDTLLIARTPEVTCVVVDLDPEDPEVQISWFVDGK | |
| | QMQTAKTQPREEQFNGTYRVVSVLPIGHQDWLKGK | |
| | QFTCKVNNKALPSPIERTISKARGQAHQPSVYVLP | |
| | PSREELSKNTVSLTCLIKDFFPPDIDVEWQSNGQQ | |
| | EPESKYRTTPPQLDEDGSYFLYSKLSVDKSRWQRG | |
| | DTFICAVMHEALHNHYTQESLSHSPGK | |
| 134 | SGSVKVLHEPSCFSDYISTSVCQWKMDHPTNCSAE | Exemplary canine |
| | LRLSYQLDEMGSENHTCVPENREDSVCVCSMPIDD | IL4RECD-IL13RECD-IgGB |
| | AVEADVYQLDLWAGQQLLWSGSFQPSKHVKPRTPG | Fc (without signal sequence) |
| | NLTVHPNISHTWLLMWTNPYPTENHLHSELTYMVN | Flexible linker GGGSG may |
| | VSNDNDPEDFKVYNVTYMGPTLRLAASTLKSGASY | be extended |
| | SARVRAWAQTYNSTWSDWSPSTTWLNYYEPGGGSG | |
| | GGGSGGGGSGGGGSGTETQPPVTNLSVSVENLCTV | |
| | IWTWDPPEGASPNCTLRYFSHFDNKQDKKIAPETH | |
| | RSKEVPLNERICLQVGSQCSTNESDNPSILVEKCT | |
| | PPPEGDPESAVTELQCVWHNLSYMKCTWLPGRNTS | |
| | PDTNYTLYYWHSSLGKILQCEDIYREGQHIGCSFA | |
| | LTNLKDSSFEQHSVQIVVKDNAGKIRPSFNIVPLT | |
| | SHVKPDPPHIKRLFFQNGNLYVQWKNPQNFYSRCL | |
| | SYQVEVNNSQTETNDIFYVEEAKCQNSEFEGNLEG | |
| | TICFMVPGVLPDTLNTVRIRVRTNKLCYEDDKLWS | |
| | NWSQAMSIGENTDPTPKRENGRVPRPPDCPKCPAP | |
| | EMLGGPSVFIFPPKPKDTLLIARTPEVTCVVVDLD | |
| | PEDPEVQISWFVDGKQMQTAKTQPREEQFNGTYRV | |
| | VSVLPIGHQDWLKGKQFTCKVNNKALPSPIERTIS | |
| | KARGQAHQPSVYVLPPSREELSKNTVSLTCLIKDE | |
| | FPPDIDVEWQSNGQQEPESKYRTTPPQLDEDGSYF | |
| | LYSKLSVDKSRWQRGDTFICAVMHEALHNHYTQES | |
| | LSHSPGK | |
| 135 | SGSVKVLHEPSCFSDYISTSVCQWKMDHPTNCSAE | Exemplary canine |
| | LRLSYQLDEMGSENHTCVPENREDSVCVCSMPIDD | IL4RECD-IL13RECD- |
| | AVEADVYQLDLWAGQQLLWSGSFQPSKHVKPRTPG | variant IgGB Fc |
| | NLTVHPNISHTWLLMWTNPYPTENHLHSELTYMVN | (F00)(without signal |
| | VSNDNDPEDFKVYNVTYMGPTLRLAASTLKSGASY | sequence) |
| | SARVRAWAQTYNSTWSDWSPSTTWLNYYEPGGGSG | |
| | TETQPPVTNLSVSVENLCTVIWTWDPPEGASPNCT | |
| | LRYFSHFDNKQDKKIAPETHRSKEVPLNERICLQV | |
| | GSQCSTNESDNPSILVEKCTPPPEGDPESAVTELQ | |
| | CVWHNLSYMKCTWLPGRNTSPDTNYTLYYWHSSLG | |
| | KILQCEDIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETND | |
| | IFYVEEAKCQNSEFEGNLEGTICFMVPGVLPDTLN | |
| | TVRIRVRTNKLCYEDDKLWSNWSQAMSIGENTDPT | |
| | PKRENGRVPRPPDCPKCPAPEMLGGPSVFIFPPKP | |
| | KDTLFIARTPEVTCVVVDLDPEDPEVQISWFVDGK | |
| | QMQTAKTQPREEQFNGTYRVVSVLPIGHQDWLKGK | |
| | QFTCKVNNKALPSPIERTISKARGQAHQPSVYVLP | |
| | PSREELSKNTVSLTCLIKDFFPPDIDVEWQSNGQQ | |
| | EPESKYRTTPPQLDEDGSYFLYSKLSVDKSRWQRG | |
| | DTFICAVMHEALHNHYTQESLSHSPGK | |
| 136 | SGSVKVLHEPSCFSDYISTSVCQWKMDHPTNCSAE | Exemplary canine |
| | LRLSYQLDFMGSENHTCVPENREDSVCVCSMPIDD | IL4RECD-IL13RECD- |
| | AVEADVYQLDLWAGQQLLWSGSFQPSKHVKPRTPG | variant IgGB Fc |
| | NLTVHPNISHTWLLMWTNPYPTENHLHSELTYMVN | (Y00)(without signal |
| | VSNDNDPEDFKVYNVTYMGPTLRLAASTLKSGASY | sequence) |
| | SARVRAWAQTYNSTWSDWSPSTTWLNYYEPGGGSG | |
| | TETQPPVTNLSVSVENLCTVIWTWDPPEGASPNCT | |
| | LRYFSHFDNKQDKKIAPETHRSKEVPLNERICLQV | |
| | GSQCSTNESDNPSILVEKCTPPPEGDPESAVTELQ | |
| | CVWHNLSYMKCTWLPGRNTSPDTNYTLYYWHSSLG | |
| | KILQCEDIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETND | |
| | IFYVEEAKCQNSEFEGNLEGTICFMVPGVLPDTLN | |
| | TVRIRVRTNKLCYEDDKLWSNWSQAMSIGENTDPT | |
| | PKRENGRVPRPPDCPKCPAPEMLGGPSVFIFPPKP | |
| | KDTLYIARTPEVTCVVVDLDPEDPEVQISWFVDGK | |
| | QMQTAKTQPREEQFNGTYRVVSVLPIGHQDWLKGK | |
| | QFTCKVNNKALPSPIERTISKARGQAHQPSVYVLP | |
| | PSREELSKNTVSLTCLIKDEFPPDIDVEWQSNGQQ | |
| | EPESKYRTTPPQLDEDGSYFLYSKLSVDKSRWQRG | |
| | DTFICAVMHEALHNHYTQESLSHSPGK | |
| 137 | SGSVKVLHEPSCFSDYISTSVCQWKMDHPTNCSAE | Exemplary canine |
| | LRLSYQLDFMGSENHTCVPENREDSVCVCSMPIDD | IL4RECD-IL13RECD- |
| | AVEADVYQLDLWAGQQLLWSGSFQPSKHVKPRTPG | variant IgGB Fc (0Y0) |
| | NLTVHPNISHTWLLMWTNPYPTENHLHSELTYMVN | (without signal sequence) |
| | VSNDNDPEDFKVYNVTYMGPTLRLAASTLKSGASY | |
| | SARVRAWAQTYNSTWSDWSPSTTWLNYYEPGGGSG | |
| | TETQPPVTNLSVSVENLCTVIWTWDPPEGASPNCT | |
| | LRYFSHFDNKQDKKIAPETHRSKEVPLNERICLQV | |
| | GSQCSTNESDNPSILVEKCTPPPEGDPESAVTELQ | |
| | CVWHNLSYMKCTWLPGRNTSPDTNYTLYYWHSSLG | |
| | KILQCEDIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETND | |
| | IFYVEEAKCQNSEFEGNLEGTICFMVPGVLPDTLN | |
| | TVRIRVRTNKLCYEDDKLWSNWSQAMSIGENTDPT | |
| | PKRENGRVPRPPDCPKCPAPEMLGGPSVFIFPPKP | |
| | KDTLLIARTPEVTCVVVDLDPEDPEVQISWFVDGK | |
| | QMQTAKTQPREEQFNGTYRVVSVLPIGHYDWLKGK | |
| | QFTCKVNNKALPSPIERTISKARGQAHQPSVYVLP | |
| | PSREELSKNTVSLTCLIKDFFPPDIDVEWQSNGQQ | |
| | EPESKYRTTPPQLDEDGSYFLYSKLSVDKSRWQRG | |
| | DTFICAVMHEALHNHYTQESLSHSPGK | |
| 138 | SGSVKVLHEPSCFSDYISTSVCQWKMDHPTNCSAE | Exemplary canine |
| | LRLSYQLDFMGSENHTCVPENREDSVCVCSMPIDD | IL4RECD-IL13RECD- |
| | AVEADVYQLDLWAGQQLLWSGSFQPSKHVKPRTPG | variant IgGB Fc (F00) |
| | NLTVHPNISHTWLLMWTNPYPTENHLHSELTYMVN | (without signal sequence) |
| | VSNDNDPEDFKVYNVTYMGPTLRLAASTLKSGASY | |
| | SARVRAWAQTYNSTWSDWSPSTTWLNYYEPGGGSG | |
| | TETQPPVTNLSVSVENLATVIWTWDPPEGASPNCT | |
| | LRYFSHFDNKQDKKIAPETHRSKEVPLNERICLQV | |
| | GSQCSTNESDNPSILVEKCTPPPEGDPESAVTELQ | |
| | CVWHNLSYMKCTWLPGRNTSPDTNYTLYYWHSSLG | |
| | KILQCEDIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETND | |
| | IFYVEEAKCQNSEFEGNLEGTICFMVPGVLPDTLN | |
| | TVRIRVRTNKLCYEDDKLWSNWSQAMSIGENTDPT | |
| | PKRENGRVPRPPDCPKCPAPEMLGGPSVFIFPPKP | |
| | KDTLFIARTPEVTCVVVDLDPEDPEVQISWFVDGK | |
| | QMQTAKTQPREEQFNGTYRVVSVLPIGHQDWLKGK | |
| | QFTCKVNNKALPSPIERTISKARGQAHQPSVYVLP | |
| | PSREELSKNTVSLTCLIKDFFPPDIDVEWQSNGQQ | |
| | EPESKYRTTPPQLDEDGSYFLYSKLSVDKSRWQRG | |
| | DTFICAVMHEALHNHYTQESLSHSPGK | |
| 139 | SGSVKVLHEPSCFSDYISTSVCQWKMDHPTNCSAE | Exemplary canine |
| | LRLSYQLDEMGSENHTCVPENREDSVCVCSMPIDD | IL4RECD-IL13RECD- |
| | AVEADVYQLDLWAGQQLLWSGSFQPSKHVKPRTPG | variant IgGB Fc (Y00) |
| | NLTVHPNISHTWLLMWTNPYPTENHLHSELTYMVN | (without signal sequence) |
| | VSNDNDPEDFKVYNVTYMGPTLRLAASTLKSGASY | |
| | SARVRAWAQTYNSTWSDWSPSTTWLNYYEPGGGSG | |
| | TETQPPVTNLSVSVENLATVIWTWDPPEGASPNCT | |
| | LRYFSHFDNKQDKKIAPETHRSKEVPLNERICLQV | |
| | GSQCSTNESDNPSILVEKCTPPPEGDPESAVTELQ | |
| | CVWHNLSYMKCTWLPGRNTSPDTNYTLYYWHSSLG | |
| | KILQCEDIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETND | |
| | IFYVEEAKCQNSEFEGNLEGTICFMVPGVLPDTLN | |
| | TVRIRVRTNKLCYEDDKLWSNWSQAMSIGENTDPT | |
| | PKRENGRVPRPPDCPKCPAPEMLGGPSVFIFPPKP | |
| | KDTLYIARTPEVTCVVVDLDPEDPEVQISWFVDGK | |
| | QMQTAKTQPREEQFNGTYRVVSVLPIGHQDWLKGK | |
| | QFTCKVNNKALPSPIERTISKARGQAHQPSVYVLP | |
| | PSREELSKNTVSLTCLIKDFFPPDIDVEWQSNGQQ | |
| | EPESKYRTTPPQLDEDGSYFLYSKLSVDKSRWQRG | |
| | DTFICAVMHEALHNHYTQESLSHSPGK | |
| 140 | SGSVKVLHEPSCFSDYISTSVCQWKMDHPTNCSAE | Exemplary canine |
| | LRLSYQLDEMGSENHTCVPENREDSVCVCSMPIDD | IL4RECD-IL13RECD- |
| | AVEADVYQLDLWAGQQLLWSGSFQPSKHVKPRTPG | variant IgGB Fc (0Y0) |
| | NLTVHPNISHTWLLMWTNPYPTENHLHSELTYMVN | (without signal sequence) |
| | VSNDNDPEDFKVYNVTYMGPTLRLAASTLKSGASY | |
| | SARVRAWAQTYNSTWSDWSPSTTWLNYYEPGGGSG | |
| | TETQPPVTNLSVSVENLATVIWTWDPPEGASPNCT | |
| | LRYFSHFDNKQDKKIAPETHRSKEVPLNERICLQV | |
| | GSQCSTNESDNPSILVEKCTPPPEGDPESAVTELQ | |
| | CVWHNLSYMKCTWLPGRNTSPDTNYTLYYWHSSLG | |
| | KILQCEDIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETND | |
| | IFYVEEAKCQNSEFEGNLEGTICFMVPGVLPDTLN | |
| | TVRIRVRTNKLCYEDDKLWSNWSQAMSIGENTDPT | |
| | PKRENGRVPRPPDCPKCPAPEMLGGPSVFIFPPKP | |
| | KDTLLIARTPEVTCVVVDLDPEDPEVQISWFVDGK | |
| | QMQTAKTQPREEQFNGTYRVVSVLPIGHYDWLKGK | |
| | QFTCKVNNKALPSPIERTISKARGQAHQPSVYVLP | |
| | PSREELSKNTVSLTCLIKDFFPPDIDVEWQSNGQQ | |
| | EPESKYRTTPPQLDEDGSYFLYSKLSVDKSRWQRG | |
| | DTFICAVMHEALHNHYTQESLSHSPGK | |
| 141 | SGSVKVLHEPSCESDYISTSVCQWKMDHPTNCSAE | Exemplary canine |
| | LRLSYQLDFMGSENHTCVPENREDSVCVCSMPIDD | IL4RECD-IL13RECD- |
| | AVEADVYQLDLWAGQQLLWSGSFQPSKHVKPRTPG | variant IgGB Fc (F00) |
| | NLTVHPNISHTWLLMWTNPYPTENHLHSELTYMVN | (without signal sequence) |
| | VSNDNDPEDFKVYNVTYMGPTLRLAASTLKSGASY | |
| | SARVRAWAQTYNSTWSDWSPSTTWLNYYEPGGGSG | |
| | TETQPPVTNLSVSVENLVTVIWTWDPPEGASPNCT | |
| | LRYFSHFDNKQDKKIAPETHRSKEVPLNERICLQV | |
| | GSQCSTNESDNPSILVEKCTPPPEGDPESAVTELQ | |
| | CVWHNLSYMKCTWLPGRNTSPDTNYTLYYWHSSLG | |
| | KILQCEDIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETND | |
| | IFYVEEAKCQNSEFEGNLEGTICFMVPGVLPDTLN | |
| | TVRIRVRTNKLCYEDDKLWSNWSQAMSIGENTDPT | |
| | PKRENGRVPRPPDCPKCPAPEMLGGPSVFIFPPKP | |
| | KDTLFIARTPEVTCVVVDLDPEDPEVQISWFVDGK | |
| | QMQTAKTQPREEQFNGTYRVVSVLPIGHQDWLKGK | |
| | QFTCKVNNKALPSPIERTISKARGQAHQPSVYVLP | |
| | PSREELSKNTVSLTCLIKDFFPPDIDVEWQSNGQQ | |
| | EPESKYRTTPPQLDEDGSYFLYSKLSVDKSRWQRG | |
| | DTFICAVMHEALHNHYTQESLSHSPGK | |
| 142 | SGSVKVLHEPSCFSDYISTSVCQWKMDHPTNCSAE | Exemplary canine |
| | LRLSYQLDEMGSENHTCVPENREDSVCVCSMPIDD | IL4RECD-IL13RECD- |
| | AVEADVYQLDLWAGQQLLWSGSFQPSKHVKPRTPG | variant IgGB Fc (Y00) |
| | NLTVHPNISHTWLLMWTNPYPTENHLHSELTYMVN | (without signal sequence) |
| | VSNDNDPEDFKVYNVTYMGPTLRLAASTLKSGASY | |
| | SARVRAWAQTYNSTWSDWSPSTTWLNYYEPGGGSG | |
| | TETQPPVTNLSVSVENLVTVIWTWDPPEGASPNCT | |
| | LRYFSHFDNKQDKKIAPETHRSKEVPLNERICLQV | |
| | GSQCSTNESDNPSILVEKCTPPPEGDPESAVTELQ | |
| | CVWHNLSYMKCTWLPGRNTSPDTNYTLYYWHSSLG | |
| | KILQCEDIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETND | |
| | IFYVEEAKCQNSEFEGNLEGTICFMVPGVLPDTLN | |
| | TVRIRVRTNKLCYEDDKLWSNWSQAMSIGENTDPT | |
| | PKRENGRVPRPPDCPKCPAPEMLGGPSVFIFPPKP | |
| | KDTLYIARTPEVTCVVVDLDPEDPEVQISWFVDGK | |
| | QMQTAKTQPREEQFNGTYRVVSVLPIGHQDWLKGK | |
| | QFTCKVNNKALPSPIERTISKARGQAHQPSVYVLP | |
| | PSREELSKNTVSLTCLIKDFFPPDIDVEWQSNGQQ | |
| | EPESKYRTTPPQLDEDGSYFLYSKLSVDKSRWQRG | |
| | DTFICAVMHEALHNHYTQESLSHSPGK | |
| 143 | SGSVKVLHEPSCFSDYISTSVCQWKMDHPINCSAE | Exemplary canine |
| | LRLSYQLDEMGSENHTCVPENREDSVCVCSMPIDD | IL4RECD-IL13RECD- |
| | AVEADVYQLDLWAGQQLLWSGSFQPSKHVKPRTPG | variant IgGB Fc (0Y0) |
| | NLTVHPNISHTWLLMWTNPYPTENHLHSELTYMVN | (without signal sequence) |
| | VSNDNDPEDFKVYNVTYMGPTLRLAASTLKSGASY | |
| | SARVRAWAQTYNSTWSDWSPSTTWLNYYEPGGGSG | |
| | TETQPPVTNLSVSVENLVTVIWTWDPPEGASPNCT | |
| | LRYFSHFDNKQDKKIAPETHRSKEVPLNERICLQV | |
| | GSQCSTNESDNPSILVEKCTPPPEGDPESAVTELQ | |
| | CVWHNLSYMKCTWLPGRNTSPDTNYTLYYWHSSLG | |
| | KILQCEDIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETND | |
| | IFYVEEAKCQNSEFEGNLEGTICFMVPGVLPDTLN | |
| | TVRIRVRTNKLCYEDDKLWSNWSQAMSIGENTDPT | |
| | PKRENGRVPRPPDCPKCPAPEMLGGPSVFIFPPKP | |
| | KDTLLIARTPEVTCVVVDLDPEDPEVQISWFVDGK | |
| | QMQTAKTQPREEQFNGTYRVVSVLPIGHYDWLKGK | |
| | QFTCKVNNKALPSPIERTISKARGQAHQPSVYVLP | |
| | PSREELSKNTVSLTCLIKDFFPPDIDVEWQSNGQQ | |
| | EPESKYRTTPPQLDEDGSYFLYSKLSVDKSRWQRG | |
| | DTFICAVMHEALHNHYTQESLSHSPGK | |
| 144 | SGSVKVLHEPSCFSDYISTSVCQWKMDHPTNCSAE | Exemplary canine |
| | LRLSYQLDFMGSENHTCVPENREDSVCVCSMPIDD | IL4RECD-IL13RECD- |
| | AVEADVYQLDLWAGQQLLWSGSFQPSKHVKPRTPG | variant IgGB Fc (F00) |
| | NLTVHPNISHTWLLMWTNPYPTENHLHSELTYMVN | |
| | VSNDNDPEDFKVYNVTYMGPTLRLAASTLKSGASY | (without signal sequence) |
| | SARVRAWAQTYNSTWSDWSPSTTWLNYYEPGGGSG | |
| | TETQPPVTNLSVSVENLSTVIWTWDPPEGASPNCT | |
| | LRYFSHFDNKQDKKIAPETHRSKEVPLNERICLQV | |
| | GSQCSTNESDNPSILVEKCTPPPEGDPESAVTELQ | |
| | CVWHNLSYMKCTWLPGRNTSPDTNYTLYYWHSSLG | |
| | KILQCEDIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETND | |
| | IFYVEEAKCQNSEFEGNLEGTICFMVPGVLPDTLN | |
| | TVRIRVRTNKLCYEDDKLWSNWSQAMSIGENTDPT | |
| | PKRENGRVPRPPDCPKCPAPEMLGGPSVFIFPPKP | |
| | KDTLFIARTPEVTCVVVDLDPEDPEVQISWFVDGK | |
| | QMQTAKTQPREEQFNGTYRVVSVLPIGHQDWLKGK | |
| | QFTCKVNNKALPSPIERTISKARGQAHQPSVYVLP | |
| | PSREELSKNTVSLTCLIKDFFPPDIDVEWQSNGQQ | |
| | EPESKYRTTPPQLDEDGSYFLYSKLSVDKSRWQRG | |
| | DTFICAVMHEALHNHYTQESLSHSPGK | |
| 145 | SGSVKVLHEPSCFSDYISTSVCQWKMDHPTNCSAE | Exemplary canine |
| | LRLSYQLDEMGSENHTCVPENREDSVCVCSMPIDD | IL4RECD-IL13RECD- |
| | AVEADVYQLDLWAGQQLLWSGSFQPSKHVKPRTPG | variant IgGB Fc (Y00) |
| | NLTVHPNISHTWLLMWTNPYPTENHLHSELTYMVN | (without signal sequence) |
| | VSNDNDPEDFKVYNVTYMGPTLRLAASTLKSGASY | |
| | SARVRAWAQTYNSTWSDWSPSTTWLNYYEPGGGSG | |
| | TETQPPVTNLSVSVENLSTVIWTWDPPEGASPNCT | |
| | LRYFSHFDNKQDKKIAPETHRSKEVPLNERICLQV | |
| | GSQCSTNESDNPSILVEKCTPPPEGDPESAVTELQ | |
| | CVWHNLSYMKCTWLPGRNTSPDTNYTLYYWHSSLG | |
| | KILQCEDIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETND | |
| | IFYVEEAKCQNSEFEGNLEGTICFMVPGVLPDTLN | |
| | TVRIRVRTNKLCYEDDKLWSNWSQAMSIGENTDPT | |
| | PKRENGRVPRPPDCPKCPAPEMLGGPSVFIFPPKP | |
| | KDTLYIARTPEVTCVVVDLDPEDPEVQISWFVDGK | |
| | QMQTAKTQPREEQFNGTYRVVSVLPIGHQDWLKGK | |
| | QFTCKVNNKALPSPIERTISKARGQAHQPSVYVLP | |
| | PSREELSKNTVSLTCLIKDFFPPDIDVEWQSNGQQ | |
| | EPESKYRTTPPQLDEDGSYFLYSKLSVDKSRWQRG | |
| | DTFICAVMHEALHNHYTQESLSHSPGK | |
| 146 | SGSVKVLHEPSCFSDYISTSVCQWKMDHPTNCSAE | Exemplary canine |
| | LRLSYQLDEMGSENHTCVPENREDSVCVCSMPIDD | IL4RECD-IL13RECD- |
| | AVEADVYQLDLWAGQQLLWSGSFQPSKHVKPRTPG | variant IgGB Fc (0Y0) |
| | NLTVHPNISHTWLLMWTNPYPTENHLHSELTYMVN | (without signal sequence) |
| | VSNDNDPEDFKVYNVTYMGPTLRLAASTLKSGASY | |
| | SARVRAWAQTYNSTWSDWSPSTTWLNYYEPGGGSG | |
| | TETQPPVTNLSVSVENLSTVIWTWDPPEGASPNCT | |
| | LRYFSHFDNKQDKKIAPETHRSKEVPLNERICLQV | |
| | GSQCSTNESDNPSILVEKCTPPPEGDPESAVTELQ | |
| | CVWHNLSYMKCTWLPGRNTSPDTNYTLYYWHSSLG | |
| | KILQCEDIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETND | |
| | IFYVEEAKCQNSEFEGNLEGTICFMVPGVLPDTLN | |
| | TVRIRVRINKLCYEDDKLWSNWSQAMSIGENTDPT | |
| | PKRENGRVPRPPDCPKCPAPEMLGGPSVFIFPPKP | |
| | KDTLLIARTPEVTCVVVDLDPEDPEVQISWFVDGK | |
| | QMQTAKTQPREEQFNGTYRVVSVLPIGHYDWLKGK | |
| | QFTCKVNNKALPSPIERTISKARGQAHQPSVYVLP | |
| | PSREELSKNTVSLTCLIKDFFPPDIDVEWQSNGQQ | |
| | EPESKYRTTPPQLDEDGSYFLYSKLSVDKSRWQRG | |
| | DTFICAVMHEALHNHYTQESLSHSPGK | |
| 147 | TETQPPVTNLSVSVENLATVIWTWDPPEGASPNCT | Exemplary variant canine |
| | LRYFSHFDNKQDKKIAPETHRSKEVPLNERICLQV | IL13R extracellular domain |
| | GSQCSTNESDNPSILVEKCTPPPEGDPESAVTELQ | (without signal sequence) |
| | CVWHNLSYMKCTWLPGRNTSPDTNYTLYYWHSSLG | |
| | KILQCEDIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETND | |
| | IFYVEEAKCQNSEFEGNLEGTICFMVPGVLPDTLN | |
| | TVRIRVRTNKLCYEDDKLWSNWSQAMSIGENTDPT | |
| 148 | TETQPPVTNLSVSVENLVTVIWTWDPPEGASPNCT | Exemplary variant canine |
| | LRYFSHFDNKQDKKIAPETHRSKEVPLNERICLQV | IL13R extracellular domain |
| | GSQCSTNESDNPSILVEKCTPPPEGDPESAVTELQ | (without signal sequence) |
| | CVWHNLSYMKCTWLPGRNTSPDTNYTLYYWHSSLG | |
| | KILQCEDIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETND | |
| | IFYVEEAKCQNSEFEGNLEGTICFMVPGVLPDTLN | |
| | TVRIRVRTNKLCYEDDKLWSNWSQAMSIGENTDPT | |
| 149 | TETQPPVTNLSVSVENLSTVIWTWDPPEGASPNCT | Exemplary variant canine |
| | LRYFSHFDNKQDKKIAPETHRSKEVPLNERICLQV | IL13R extracellular domain |
| | GSQCSTNESDNPSILVEKCTPPPEGDPESAVTELQ | (without signal sequence) |
| | CVWHNLSYMKCTWLPGRNTSPDTNYTLYYWHSSLG | |
| | KILQCEDIYREGQHIGCSFALTNLKDSSFEQHSVQ | |
| | IVVKDNAGKIRPSFNIVPLTSHVKPDPPHIKRLFF | |
| | QNGNLYVQWKNPQNFYSRCLSYQVEVNNSQTETND | |
| | IFYVEEAKCQNSEFEGNLEGTICFMVPGVLPDTLN | |
| | TVRIRVRTNKLCYEDDKLWSNWSQAMSIGENTDPT | |
| 164 | MAFIHLDVGFLYTLLVCTAFGSMLSNAEIKVNPPQ | Canis lupus |
| | DFEIVDPGYLGYLSLQWQPPLFPDNFKECTIEYEL | interleukin-13 receptor |
| | KYRNIDSENWKTIITKNLHYKDGFDLNKGIEAKIN | subunit alpha-2 precursor |
| | TLLPAQCTNGSEVRSSWAETTYWTSPQGNRETKIQ | |
| | DMDCVYYNWQYLVCSWKPGMGVHFDTNYQLFYWYE | |
| | GLDHSAECTDYIKVNGKNMGCRFPYLESSDYKDFY | |
| | ICVNGSSESQPIRPSYFIFQLQNIVKPMPPDYLSL | |
| | TVKNSEEINLKWNMPKGPIPAKCFIYEIEFTEDGT | |
| | TWVTTTVENEIQITRTSNESQKLCFLVRSKVNIYC | |
| | SDDGIWSEWSDEQCWKGDIWKETLVFFLIPFAFVS | |
| | IFVLVITCLLLYKQRALLKTIFHTKKEVFSHQDTF | |
| | C | |
| 165 | MAFVHLDVLCFYSLLICTAFSSVSSNAEIKVNPPQ | Felis catus |
| | DFEIVDPGYLGYLCLQWQPPLFLDKFEECTVEYEL | interleukin-13 receptor |
| | KYRNIDSEDWKTIITKNLHYNDGFDLNKGVEAKIH | subunit alpha-2 isoform X1 |
| | TLLPPHCTNGSEVQSLWSEATYWKSPQGSQETKIQ | |
| | EMDCVYYNWEYLLCSWKPGLGVHFHTSYQLFYWYD | |
| | GLDHATQCPDYIKVDGQNIGCRFPHLEASDYKDFY | |
| | ICVNGSSDSYPIRPSYFIFQLQNIVKPLPPDYLSL | |
| | TVKNSEEVNLKWSMPQGPIPAKCFIYEIEFTEDDT | |
| | TWVTTTVENEIRVARISNESQQLCFLVRSKVNIYC | |
| | SDDGIWSEWSDE | |
| 166 | MRGKSGNFKILNLGEMALTRLDSRCLYTLLICMAF | Equus caballus |
| | GSTLSSNAEINVNAPQDFEIVDPGYLGYLYLQWQR | interleukin-13 receptor |
| | PLSLDNFKECTVEYELKYRNIDSENWKTIITKNLC | subunit alpha-2 isoform X1 |
| | YKDGFDLNKGVEAKIRTLLPGQCTNGSEVQSSWAE | |
| | VTYWTSLQGNLGTKIQDMDCIYYNWQDLLCSWKSG | |
| | MGVHFDTNYNLFYWYEGLHHALQCADYIKVNGKNI | |
| | GCRFPYLESSDYKDFYICVNGSSESEPIRPSYFIF | |
| | QLQNIVKPLPPDYLSLIVKSSEDISLKWNMPRGPI | |
| | PAKCFIYEIKFTEDDTTWVTTTVENEIYIARTSNE | |
| | SKRLCFLVRSKVNIYCSDDGIWSEWSDEQCWNGDI | |
| | LKKASLFFLIPFALISLLVSLVTCLVLYNQKDLLK | |
| | TAFQTKKEVFSHQETQC | |
| 167 | KVNPPQDFEIVDPGYLGYLSLQWQPPLFPDNEKEC | Exemplary canine IL13R |
| | TIEYELKYRNIDSENWKTIITKNLHYKDGFDLNKG | decoy extracellular domain |
| | IEAKINTLLPAQCTNGSEVRSSWAETTYWTSPQGN | (ECD; without signal |
| | RETKIQDMDCVYYNWQYLVCSWKPGMGVHEDTNYQ | sequence) |
| | LFYWYEGLDHSAECTDYIKVNGKNMGCRFPYLESS | |
| | DYKDFYICVNGSSESQPIRPSYFIFQLQNIVKPMP | |
| | PDYLSLTVKNSEEINLKWNMPKGPIPAKCFIYEIE | |
| | FTEDGTTWVTTTVENEIQITRTSNESQKLCFLVRS | |
| | KVNIYCSDDGIWSEWSDE | |
| 168 | KVNPPQDFEIVDPGYLGYLCLQWQPPLFLDKFEEC | Exemplary feline IL13R |
| | TVEYELKYRNIDSEDWKTIITKNLHYNDGFDLNKG | decoy extracellular domain |
| | VEAKIHTLLPPHCTNGSEVQSLWSEATYWKSPQGS | (ECD; without signal |
| | QETKIQEMDCVYYNWEYLLCSWKPGLGVHFHTSYQ | sequence) |
| | LFYWYDGLDHATQCPDYIKVDGQNIGCRFPHLEAS | |
| | DYKDFYICVNGSSDSYPIRPSYFIFQLQNIVKPLP | |
| | PDYLSLTVKNSEEVNLKWSMPQGPIPAKCFIYEIE | |
| | FTEDDTTWVTTTVENEIRVARISNESQQLCELVRS | |
| | KVNIYCSDDGIWSEWSDE | |
| 169 | NVNAPQDFEIVDPGYLGYLYLQWQRPLSLDNFKEC | Exemplary equine IL13R |
| | TVEYELKYRNIDSENWKTIITKNLCYKDGFDLNKG | decoy extracellular domain |
| | VEAKIRTLLPGQCTNGSEVQSSWAEVTYWTSLQGN | (ECD; without signal |
| | LGTKIQDMDCIYYNWQDLLCSWKSGMGVHEDTNYN | sequence) |
| | LFYWYEGLHHALQCADYIKVNGKNIGCRFPYLESS | |
| | DYKDFYICVNGSSESEPIRPSYFIFQLQNIVKPLP | |
| | PDYLSLIVKSSEDISLKWNMPRGPIPAKCFIYEIK | |
| | FTEDDTTWVTTTVENEIYIARTSNESKRLCFLVRS | |
| | KVNIYCSDDGIWSEWSDE | |
| 170 | KVNPPQDFEIVDPGYLGYLSLQWQPPLFPDNEKEC | Exemplary canine IL13Rd |
| | TIEYELKYRNIDSENWKTIITKNLHYKDGFDLNKG | ECD-IL4RECD-IgGB Fc |
| | IEAKINTLLPAQCTNGSEVRSSWAETTYWTSPQGN | (without signal sequence) |
| | RETKIQDMDCVYYNWQYLVCSWKPGMGVHEDTNYQ | |
| | LFYWYEGLDHSAECTDYIKVNGKNMGCRFPYLESS | |
| | DYKDFYICVNGSSESQPIRPSYFIFQLQNIVKPMP | |
| | PDYLSLTVKNSEEINLKWNMPKGPIPAKCFIYEIE | |
| | FTEDGTTWVTTTVENEIQITRTSNESQKLCFLVRS | |
| | KVNIYCSDDGIWSEWSDEGGGSGSGSVKVLHEPSC | |
| | FSDYISTSVCQWKMDHPTNCSAELRLSYQLDEMGS | |
| | ENHTCVPENREDSVCVCSMPIDDAVEADVYQLDLW | |
| | AGQQLLWSGSFQPSKHVKPRTPGNLTVHPNISHTW | |
| | LLMWTNPYPTENHLHSELTYMVNVSNDNDPEDFKV | |
| | YNVTYMGPTLRLAASTLKSGASYSARVRAWAQTYN | |
| | STWSDWSPSTTWLNYYEPKRENGRVPRPPDCPKCP | |
| | APEMLGGPSVFIFPPKPKDTLLIARTPEVTCVVVD | |
| | LDPEDPEVQISWFVDGKQMQTAKTQPREEQENGTY | |
| | RVVSVLPIGHQDWLKGKQFTCKVNNKALPSPIERT | |
| | ISKARGQAHQPSVYVLPPSREELSKNTVSLTCLIK | |
| | DFFPPDIDVEWQSNGQQEPESKYRTTPPQLDEDGS | |
| | YFLYSKLSVDKSRWQRGDTFICAVMHEALHNHYTQ | |
| | ESLSHSPGK | |
| 171 | SGSVKVLHEPSCFSDYISTSVCQWKMDHPTNCSAE | Exemplary canine IL4RECD |
| | LRLSYQLDFMGSENHTCVPENREDSVCVCSMPIDD | -IL13Rd ECD-IgGB Fc |
| | AVEADVYQLDLWAGQQLLWSGSFQPSKHVKPRTPG | (without signal sequence) |
| | NLTVHPNISHTWLLMWTNPYPTENHLHSELTYMVN | |
| | VSNDNDPEDFKVYNVTYMGPTLRLAASTLKSGASY | |
| | SARVRAWAQTYNSTWSDWSPSTTWLNYYEPGGGSG | |
| | KVNPPQDFEIVDPGYLGYLSLQWQPPLFPDNEKEC | |
| | TIEYELKYRNIDSENWKTIITKNLHYKDGFDLNKG | |
| | IEAKINTLLPAQCTNGSEVRSSWAETTYWTSPQGN | |
| | RETKIQDMDCVYYNWQYLVCSWKPGMGVHEDTNYQ | |
| | LFYWYEGLDHSAECTDYIKVNGKNMGCRFPYLESS | |
| | DYKDFYICVNGSSESQPIRPSYFIFQLQNIVKPMP | |
| | PDYLSLTVKNSEEINLKWNMPKGPIPAKCFIYEIE | |
| | FTEDGTTWVTTTVENEIQITRTSNESQKLCFLVRS | |
| | KVNIYCSDDGIWSEWSDEPKRENGRVPRPPDCPKC | |
| | PAPEMLGGPSVFIFPPKPKDTLLIARTPEVTCVVV | |
| | DLDPEDPEVQISWFVDGKQMQTAKTQPREEQFNGT | |
| | YRVVSVLPIGHQDWLKGKQFTCKVNNKALPSPIER | |
| | TISKARGQAHQPSVYVLPPSREELSKNTVSLTCLI | |
| | KDFFPPDIDVEWQSNGQQEPESKYRTTPPQLDEDG | |
| | SYFLYSKLSVDKSRWQRGDTFICAVMHEALHNHYT | |
| | QESLSHSPGK | |
| 172 | SGSVKVLHEPSCFSDYISTSVCQWKMDHPTNCSAE | Exemplary canine IL4RECD- |
| | LRLSYQLDFMGSENHTCVPENREDSVCVCSMPIDD | IgGB Fc-IL13Rd ECD |
| | AVEADVYQLDLWAGQQLLWSGSFQPSKHVKPRTPG | (without signal sequence) |
| | NLTVHPNISHTWLLMWTNPYPTENHLHSELTYMVN | |
| | VSNDNDPEDFKVYNVTYMGPTLRLAASTLKSGASY | |
| | SARVRAWAQTYNSTWSDWSPSTTWLNYYEPKRENG | |
| | RVPRPPDCPKCPAPEMLGGPSVFIFPPKPKDTLLI | |
| | ARTPEVTCVVVDLDPEDPEVQISWFVDGKQMQTAK | |
| | TQPREEQFNGTYRVVSVLPIGHQDWLKGKQFTCKV | |
| | NNKALPSPIERTISKARGQAHQPSVYVLPPSREEL | |
| | SKNTVSLTCLIKDFFPPDIDVEWQSNGQQEPESKY | |
| | RTTPPQLDEDGSYFLYSKLSVDKSRWQRGDTFICA | |
| | VMHEALHNHYTQESLSHSPGKGGGSGKVNPPQDFE | |
| | IVDPGYLGYLSLQWQPPLFPDNEKECTIEYELKYR | |
| | NIDSENWKTIITKNLHYKDGFDLNKGIEAKINTLL | |
| | PAQCTNGSEVRSSWAETTYWTSPQGNRETKIQDMD | |
| | CVYYNWQYLVCSWKPGMGVHFEDTNYQLFYWYEGLD | |
| | HSAECTDYIKVNGKNMGCRFPYLESSDYKDFYICV | |
| | NGSSESQPIRPSYFIFQLQNIVKPMPPDYLSLTVK | |
| | NSEEINLKWNMPKGPIPAKCFIYEIEFTEDGTTWV | |
| | TTTVENEIQITRTSNESQKLCFLVRSKVNIYCSDD | |
| | GIWSEWSDE | |
| 173 | KVNPPQDFEIVDPGYLGYLCLQWQPPLFLDKFEEC | Exemplary feline IL13Rd |
| | TVEYELKYRNIDSEDWKTIITKNLHYNDGFDLNKG | ECD-IL4RECD-IgG2 Fc |
| | VEAKIHTLLPPHCTNGSEVQSLWSEATYWKSPQGS | (without signal sequence) |
| | QETKIQEMDCVYYNWEYLLCSWKPGLGVHFHTSYQ | |
| | LFYWYDGLDHATQCPDYIKVDGQNIGCRFPHLEAS | |
| | DYKDFYICVNGSSDSYPIRPSYFIFQLQNIVKPLP | |
| | PDYLSLTVKNSEEVNLKWSMPQGPIPAKCFIYEIE | |
| | FTEDDTTWVTTTVENEIRVARISNESQQLCFLVRS | |
| | KVNIYCSDDGIWSEWSDEGGGSGSSGSVKVLRAPT | |
| | CFSDYFSTSVCQWNMDAPTNCSAELRLSYQLNEMG | |
| | SENRTCVPENGEGAACACSMLMDDEVEADVYQLHL | |
| | WAGTQLLWSGSFKPSSHVKPRAPGNLTVHPNVSHT | |
| | WLLRWSNPYPPENHLHAELTYMVNISSEDDPTDVS | |
| | VCASGFLCHLLGLRRVETGAPGARLPPWLCAPRPR | |
| | RVPGSQCAVISCCRWVLIALTSRGGRWRLTPGLRS | |
| | QTRYVSVAEGLFGATPRVLCPGTQAGLASAAREQM | |
| | SPDPSAFHSIDYEPSPKTASTIESKTGECPKCPVP | |
| | EIPGAPSVFIFPPKPKDTLSISRTPEVTCLVVDLG | |
| | PDDSNVQITWFVDNTEMHTAKTRPREEQFNSTYRV | |
| | VSVLPILHQDWLKGKEFKCKVNSKSLPSAMERTIS | |
| | KAKGQPHEPQVYVLPPTQEELSENKVSVTCLIKGF | |
| | HPPDIAVEWEITGQPEPENNYQTTPPQLDSDGTYF | |
| | LYSRLSVDRSHWQRGNTYTCSVSHEALHSHHTQKS | |
| | LTQSPGK | |
| 174 | NVNAPQDFEIVDPGYLGYLYLQWQRPLSLDNFKEC | Exemplary equine IL13Rd |
| | TVEYELKYRNIDSENWKTIITKNLCYKDGFDLNKG | ECD-IL4RECD-IgG2 Fc |
| | VEAKIRTLLPGQCTNGSEVQSSWAEVTYWTSLQGN | (without signal sequence) |
| | LGTKIQDMDCIYYNWQDLLCSWKSGMGVHEDTNYN | |
| | LFYWYEGLHHALQCADYIKVNGKNIGCRFPYLESS | |
| | DYKDFYICVNGSSESEPIRPSYFIFQLQNIVKPLP | |
| | PDYLSLIVKSSEDISLKWNMPRGPIPAKCFIYEIK | |
| | FTEDDTTWVTTTVENEIYIARTSNESKRLCFLVRS | |
| | KVNIYCSDDGIWSEWSDEGGGSGSSGSVKVLHLTA | |
| | CFSDYISASTCEWKMDRPTNCSAQLRLSYQLNDEF | |
| | SDNLTCIPENREDEVCVCRMLMDNIVSEDVYELDL | |
| | WAGNQLLWNSSFKPSRHVKPRAPQNLTVHAISHTW | |
| | LLTWSNPYPLKNHLWSELTYLVNISKEDDPTDEKI | |
| | YNVTYMDPTLRVTASTLKSRATYSARVKARAQNYN | |
| | STWSEWSPSTTWHNYYEQPDMSKCPKCPAPELLGG | |
| | PSVFIFPPNPKDTLMISRTPVVTCVVVNLSDQYPD | |
| | VQFSWYVDNTEVHSAITKQREAQFNSTYRVVSVLP | |
| | IQHQDWLSGKEFKCSVTNVGVPQPISRAISRGKGP | |
| | SRVPQVYVLPPHPDELAKSKVSVTCLVKDFYPPDI | |
| | SVEWQSNRWPELEGKYSTTPAQLDGDGSYFLYSKL | |
| | SLETSRWQQVESFTCAVMHEALHNHYTKTDISESL | |
| | GK | |
| 175 | KVNPPQDFEIVDPGYLGYLSLQWQPPLFPDNFKEC | Exemplary canine IL13Rd |
| | TIEYELKYRNIDSENWKTIITKNLHYKDGFDLNKG | ECD-IL4RECD-variant |
| | IEAKINTLLPAQCTNGSEVRSSWAETTYWTSPQGN | IgGB Fc (F00) |
| | RETKIQDMDCVYYNWQYLVCSWKPGMGVHFDTNYQ | (without signal sequence) |
| | LFYWYEGLDHSAECTDYIKVNGKNMGCRFPYLESS | |
| | DYKDFYICVNGSSESQPIRPSYFIFQLQNIVKPMP | |
| | PDYLSLTVKNSEEINLKWNMPKGPIPAKCFIYEIE | |
| | FTEDGTTWVTTTVENEIQITRTSNESQKLCFLVRS | |
| | KVNIYCSDDGIWSEWSDEGGGSGSGSVKVLHEPSC | |
| | FSDYISTSVCQWKMDHPTNCSAELRLSYQLDEMGS | |
| | ENHTCVPENREDSVCVCSMPIDDAVEADVYQLDLW | |
| | AGQQLLWSGSFQPSKHVKPRTPGNLTVHPNISHTW | |
| | LLMWTNPYPTENHLHSELTYMVNVSNDNDPEDFKV | |
| | YNVTYMGPTLRLAASTLKSGASYSARVRAWAQTYN | |
| | STWSDWSPSTTWLNYYEPKRENGRVPRPPDCPKCP | |
| | APEMLGGPSVFIFPPKPKDTLFIARTPEVTCVVVD | |
| | LDPEDPEVQISWFVDGKQMQTAKTQPREEQFNGTY | |
| | RVVSVLPIGHQDWLKGKQFTCKVNNKALPSPIERT | |
| | ISKARGQAHQPSVYVLPPSREELSKNTVSLTCLIK | |
| | DFFPPDIDVEWQSNGQQEPESKYRTTPPQLDEDGS | |
| | YFLYSKLSVDKSRWQRGDTFICAVMHEALHNHYTQ | |
| | ESLSHSPGK | |
| 176 | SGSVKVLHEPSCFSDYISTSVCQWKMDHPINCSAE | Exemplary canine IL4RECD- |
| | LRLSYQLDFMGSENHTCVPENREDSVCVCSMPIDD | IL13Rd ECD-variant IgGB |
| | AVEADVYQLDLWAGQQLLWSGSFQPSKHVKPRTPG | Fc (F00) |
| | NLTVHPNISHTWLLMWTNPYPTENHLHSELTYMVN | (without signal sequence) |
| | VSNDNDPEDFKVYNVTYMGPTLRLAASTLKSGASY | |
| | SARVRAWAQTYNSTWSDWSPSTTWLNYYEPGGGSG | |
| | KVNPPQDFEIVDPGYLGYLSLQWQPPLFPDNEKEC | |
| | TIEYELKYRNIDSENWKTIITKNLHYKDGFDLNKG | |
| | IEAKINTLLPAQCTNGSEVRSSWAETTYWTSPQGN | |
| | RETKIQDMDCVYYNWQYLVCSWKPGMGVHFDTNYQ | |
| | LFYWYEGLDHSAECTDYIKVNGKNMGCRFPYLESS | |
| | DYKDFYICVNGSSESQPIRPSYFIFQLQNIVKPMP | |
| | PDYLSLTVKNSEEINLKWNMPKGPIPAKCFIYEIE | |
| | FTEDGTTWVTTTVENEIQITRTSNESQKLCFLVRS | |
| | KVNIYCSDDGIWSEWSDEPKRENGRVPRPPDCPKC | |
| | PAPEMLGGPSVFIFPPKPKDTLFIARTPEVTCVVV | |
| | DLDPEDPEVQISWFVDGKQMQTAKTQPREEQFNGT | |
| | YRVVSVLPIGHQDWLKGKQFTCKVNNKALPSPIER | |
| | TISKARGQAHQPSVYVLPPSREELSKNTVSLTCLI | |
| | KDFFPPDIDVEWQSNGQQEPESKYRTTPPQLDEDG | |
| | SYFLYSKLSVDKSRWQRGDTFICAVMHEALHNHYT | |
| | QESLSHSPGK | |
| 177 | SGSVKVLHEPSCFSDYISTSVCQWKMDHPTNCSAE | Exemplary canine IL4RECD- |
| | LRLSYQLDFMGSENHTCVPENREDSVCVCSMPIDD | variant IgGB Fc (F00)- |
| | AVEADVYQLDLWAGQQLLWSGSFQPSKHVKPRTPG | IL13Rd ECD |
| | NLTVHPNISHTWLLMWTNPYPTENHLHSELTYMVN | (without signal sequence) |
| | VSNDNDPEDFKVYNVTYMGPTLRLAASTLKSGASY | |
| | SARVRAWAQTYNSTWSDWSPSTTWLNYYEPKRENG | |
| | RVPRPPDCPKCPAPEMLGGPSVFIFPPKPKDTLFI | |
| | ARTPEVTCVVVDLDPEDPEVQISWFVDGKQMQTAK | |
| | TQPREEQFNGTYRVVSVLPIGHQDWLKGKQFTCKV | |
| | NNKALPSPIERTISKARGQAHQPSVYVLPPSREEL | |
| | SKNTVSLTCLIKDFFPPDIDVEWQSNGQQEPESKY | |
| | RTTPPQLDEDGSYFLYSKLSVDKSRWQRGDTFICA | |
| | VMHEALHNHYTQESLSHSPGKGGGSGKVNPPQDFE | |
| | IVDPGYLGYLSLQWQPPLFPDNFKECTIEYELKYR | |
| | NIDSENWKTIITKNLHYKDGFDLNKGIEAKINTLL | |
| | PAQCTNGSEVRSSWAETTYWTSPQGNRETKIQDMD | |
| | CVYYNWQYLVCSWKPGMGVHFDTNYQLFYWYEGLD | |
| | HSAECTDYIKVNGKNMGCRFPYLESSDYKDFYICV | |
| | NGSSESQPIRPSYFIFQLQNIVKPMPPDYLSLTVK | |
| | NSEEINLKWNMPKGPIPAKCFIYEIEFTEDGTTWV | |
| | TTTVENEIQITRTSNESQKLCFLVRSKVNIYCSDD | |
| | GIWSEWSDE | |
| 178 | KVNPPQDFEIVDPGYLGYLSLQWQPPLFPDNEKEC | Exemplary canine IL13Rd |
| | TIEYELKYRNIDSENWKTIITKNLHYKDGFDLNKG | ECD-IL4RECD-variant |
| | IEAKINTLLPAQCTNGSEVRSSWAETTYWTSPQGN | IgGB Fc (Y00) |
| | RETKIQDMDCVYYNWQYLVCSWKPGMGVHEDTNYQ | (without signal sequence) |
| | LFYWYEGLDHSAECTDYIKVNGKNMGCRFPYLESS | |
| | DYKDFYICVNGSSESQPIRPSYFIFQLQNIVKPMP | |
| | PDYLSLTVKNSEEINLKWNMPKGPIPAKCFIYEIE | |
| | FTEDGTTWVTTTVENEIQITRTSNESQKLCFLVRS | |
| | KVNIYCSDDGIWSEWSDEGGGSGSGSVKVLHEPSC | |
| | FSDYISTSVCQWKMDHPTNCSAELRLSYQLDEMGS | |
| | ENHTCVPENREDSVCVCSMPIDDAVEADVYQLDLW | |
| | AGQQLLWSGSFQPSKHVKPRTPGNLTVHPNISHTW | |
| | LLMWTNPYPTENHLHSELTYMVNVSNDNDPEDFKV | |
| | YNVTYMGPTLRLAASTLKSGASYSARVRAWAQTYN | |
| | STWSDWSPSTTWLNYYEPKRENGRVPRPPDCPKCP | |
| | APEMLGGPSVFIFPPKPKDTLYIARTPEVTCVVVD | |
| | LDPEDPEVQISWFVDGKQMQTAKTQPREEQFNGTY | |
| | RVVSVLPIGHQDWLKGKQFTCKVNNKALPSPIERT | |
| | ISKARGQAHQPSVYVLPPSREELSKNTVSLTCLIK | |
| | DFFPPDIDVEWQSNGQQEPESKYRTTPPQLDEDGS | |
| | YFLYSKLSVDKSRWQRGDTFICAVMHEALHNHYTQ | |
| | ESLSHSPGK | |
| 179 | SGSVKVLHEPSCFSDYISTSVCQWKMDHPTNCSAE | Exemplary canine IL4RECD- |
| | LRLSYQLDFMGSENHTCVPENREDSVCVCSMPIDD | IL13Rd ECD-variant IgGB |
| | AVEADVYQLDLWAGQQLLWSGSFQPSKHVKPRTPG | Fc (Y00) |
| | NLTVHPNISHTWLLMWTNPYPTENHLHSELTYMVN | (without signal sequence) |
| | VSNDNDPEDFKVYNVTYMGPTLRLAASTLKSGASY | |
| | SARVRAWAQTYNSTWSDWSPSTTWLNYYEPGGGSG | |
| | KVNPPQDFEIVDPGYLGYLSLQWQPPLFPDNEKEC | |
| | TIEYELKYRNIDSENWKTIITKNLHYKDGFDLNKG | |
| | IEAKINTLLPAQCTNGSEVRSSWAETTYWTSPQGN | |
| | RETKIQDMDCVYYNWQYLVCSWKPGMGVHFDTNYQ | |
| | LFYWYEGLDHSAECTDYIKVNGKNMGCRFPYLESS | |
| | DYKDFYICVNGSSESQPIRPSYFIFQLQNIVKPMP | |
| | PDYLSLTVKNSEEINLKWNMPKGPIPAKCFIYEIE | |
| | FTEDGTTWVTTTVENEIQITRTSNESQKLCFLVRS | |
| | KVNIYCSDDGIWSEWSDEPKRENGRVPRPPDCPKC | |
| | PAPEMLGGPSVFIFPPKPKDTLYIARTPEVTCVVV | |
| | DLDPEDPEVQISWFVDGKQMQTAKTQPREEQENGT | |
| | YRVVSVLPIGHQDWLKGKQFTCKVNNKALPSPIER | |
| | TISKARGQAHQPSVYVLPPSREELSKNTVSLTCLI | |
| | KDFFPPDIDVEWQSNGQQEPESKYRTTPPQLDEDG | |
| | SYFLYSKLSVDKSRWQRGDTFICAVMHEALHNHYT | |
| | QESLSHSPGK | |
| 180 | SGSVKVLHEPSCFSDYISTSVCQWKMDHPTNCSAE | Exemplary canine IL4RECD- |
| | LRLSYQLDEMGSENHTCVPENREDSVCVCSMPIDD | variant IgGB Fc (Y00)- |
| | AVEADVYQLDLWAGQQLLWSGSFQPSKHVKPRTPG | IL13Rd ECD |
| | NLTVHPNISHTWLLMWTNPYPTENHLHSELTYMVN | (without signal sequence) |
| | VSNDNDPEDFKVYNVTYMGPTLRLAASTLKSGASY | |
| | SARVRAWAQTYNSTWSDWSPSTTWLNYYEPKRENG | |
| | RVPRPPDCPKCPAPEMLGGPSVFIFPPKPKDTLYI | |
| | ARTPEVTCVVVDLDPEDPEVQISWFVDGKQMQTAK | |
| | TQPREEQFNGTYRVVSVLPIGHQDWLKGKQFTCKV | |
| | NNKALPSPIERTISKARGQAHQPSVYVLPPSREEL | |
| | SKNTVSLTCLIKDFFPPDIDVEWQSNGQQEPESKY | |
| | RTTPPQLDEDGSYFLYSKLSVDKSRWQRGDTFICA | |
| | VMHEALHNHYTQESLSHSPGKGGGSGKVNPPQDFE | |
| | IVDPGYLGYLSLQWQPPLFPDNEKECTIEYELKYR | |
| | NIDSENWKTIITKNLHYKDGFDLNKGIEAKINTLL | |
| | PAQCTNGSEVRSSWAETTYWTSPQGNRETKIQDMD | |
| | CVYYNWQYLVCSWKPGMGVHEDTNYQLFYWYEGLD | |
| | HSAECTDYIKVNGKNMGCRFPYLESSDYKDFYICV | |
| | NGSSESQPIRPSYFIFQLQNIVKPMPPDYLSLTVK | |
| | NSEEINLKWNMPKGPIPAKCFIYEIEFTEDGTTWV | |
| | TTTVENEIQITRTSNESQKLCFLVRSKVNIYCSDD | |
| | GIWSEWSDE | |
| 181 | KVNPPQDFEIVDPGYLGYLSLQWQPPLFPDNFKEC | Exemplary canine IL13Rd |
| | TIEYELKYRNIDSENWKTIITKNLHYKDGFDLNKG | ECD-IL4RECD-variant |
| | IEAKINTLLPAQCTNGSEVRSSWAETTYWTSPQGN | IgGB Fc (0Y0) |
| | RETKIQDMDCVYYNWQYLVCSWKPGMGVHFDTNYQ | (without signal sequence) |
| | LFYWYEGLDHSAECTDYIKVNGKNMGCRFPYLESS | |
| | DYKDFYICVNGSSESQPIRPSYFIFQLQNIVKPMP | |
| | PDYLSLTVKNSEEINLKWNMPKGPIPAKCFIYEIE | |
| | FTEDGTTWVTTTVENEIQITRTSNESQKLCFLVRS | |
| | KVNIYCSDDGIWSEWSDEGGGSGSGSVKVLHEPSC | |
| | FSDYISTSVCQWKMDHPTNCSAELRLSYQLDEMGS | |
| | ENHTCVPENREDSVCVCSMPIDDAVEADVYQLDLW | |
| | AGQQLLWSGSFQPSKHVKPRTPGNLTVHPNISHTW | |
| | LLMWTNPYPTENHLHSELTYMVNVSNDNDPEDFKV | |
| | YNVTYMGPTLRLAASTLKSGASYSARVRAWAQTYN | |
| | STWSDWSPSTTWLNYYEPKRENGRVPRPPDCPKCP | |
| | APEMLGGPSVFIFPPKPKDTLLIARTPEVTCVVVD | |
| | LDPEDPEVQISWFVDGKQMQTAKTQPREEQFNGTY | |
| | RVVSVLPIGHYDWLKGKQFTCKVNNKALPSPIERT | |
| | ISKARGQAHQPSVYVLPPSREELSKNTVSLTCLIK | |
| | DFFPPDIDVEWQSNGQQEPESKYRTTPPQLDEDGS | |
| | YFLYSKLSVDKSRWQRGDTFICAVMHEALHNHYTQ | |
| | ESLSHSPGK | |
| 182 | SGSVKVLHEPSCFSDYISTSVCQWKMDHPTNCSAE | Exemplary canine IL4RECD- |
| | LRLSYQLDFMGSENHTCVPENREDSVCVCSMPIDD | IL13Rd ECD-variant IgGB |
| | AVEADVYQLDLWAGQQLLWSGSFQPSKHVKPRTPG | Fc (OY0) |
| | NLTVHPNISHTWLLMWTNPYPTENHLHSELTYMVN | (without signal sequence) |
| | VSNDNDPEDFKVYNVTYMGPTLRLAASTLKSGASY | |
| | SARVRAWAQTYNSTWSDWSPSTTWLNYYEPGGGSG | |
| | KVNPPQDFEIVDPGYLGYLSLQWQPPLFPDNEKEC | |
| | TIEYELKYRNIDSENWKTIITKNLHYKDGFDLNKG | |
| | IEAKINTLLPAQCTNGSEVRSSWAETTYWTSPQGN | |
| | RETKIQDMDCVYYNWQYLVCSWKPGMGVHEDTNYQ | |
| | LFYWYEGLDHSAECTDYIKVNGKNMGCRFPYLESS | |
| | DYKDFYICVNGSSESQPIRPSYFIFQLQNIVKPMP | |
| | PDYLSLTVKNSEEINLKWNMPKGPIPAKCFIYEIE | |
| | FTEDGTTWVTTTVENEIQITRTSNESQKLCFLVRS | |
| | KVNIYCSDDGIWSEWSDEPKRENGRVPRPPDCPKC | |
| | PAPEMLGGPSVFIFPPKPKDTLLIARTPEVTCVVV | |
| | DLDPEDPEVQISWFVDGKQMQTAKTQPREEQFNGT | |
| | YRVVSVLPIGHYDWLKGKQFTCKVNNKALPSPIER | |
| | TISKARGQAHQPSVYVLPPSREELSKNTVSLTCLI | |
| | KDFFPPDIDVEWQSNGQQEPESKYRTTPPQLDEDG | |
| | SYFLYSKLSVDKSRWQRGDTFICAVMHEALHNHYT | |
| | QESLSHSPGK | |
| 183 | SGSVKVLHEPSCFSDYISTSVCQWKMDHPTNCSAE | Exemplary canine IL4RECD- |
| | LRLSYQLDFMGSENHTCVPENREDSVCVCSMPIDD | variant IgGB Fc (0Y0)- |
| | AVEADVYQLDLWAGQQLLWSGSFQPSKHVKPRTPG | IL13Rd ECD |
| | NLTVHPNISHTWLLMWTNPYPTENHLHSELTYMVN | (without signal sequence) |
| | VSNDNDPEDFKVYNVTYMGPTLRLAASTLKSGASY | |
| | SARVRAWAQTYNSTWSDWSPSTTWLNYYEPKRENG | |
| | RVPRPPDCPKCPAPEMLGGPSVFIFPPKPKDTLLI | |
| | ARTPEVTCVVVDLDPEDPEVQISWFVDGKQMQTAK | |
| | TQPREEQFNGTYRVVSVLPIGHYDWLKGKQFTCKV | |
| | NNKALPSPIERTISKARGQAHQPSVYVLPPSREEL | |
| | SKNTVSLTCLIKDFFPPDIDVEWQSNGQQEPESKY | |
| | RTTPPQLDEDGSYFLYSKLSVDKSRWQRGDTFICA | |
| | VMHEALHNHYTQESLSHSPGKGGGSGKVNPPQDFE | |
| | IVDPGYLGYLSLQWQPPLFPDNEKECTIEYELKYR | |
| | NIDSENWKTIITKNLHYKDGFDLNKGIEAKINTLL | |
| | PAQCTNGSEVRSSWAETTYWTSPQGNRETKIQDMD | |
| | CVYYNWQYLVCSWKPGMGVHFDTNYQLFYWYEGLD | |
| | HSAECTDYIKVNGKNMGCRFPYLESSDYKDFYICV | |
| | NGSSESQPIRPSYFIFQLQNIVKPMPPDYLSLTVK | |
| | NSEEINLKWNMPKGPIPAKCFIYEIEFTEDGTTWV | |
| | TTTVENEIQITRTSNESQKLCFLVRSKVNIYCSDD | |
| | GIWSEWSDE | |

### DESCRIPTION

Contiguous polypeptides that bind canine IL13 and/or IL4, feline IL13 and/or IL4, and/or equine IL13 and/or IL4 are provided, for example long-acting polypeptides. In some embodiments, the contiguous polypeptides comprise an extracellular domain of an IL13R polypeptide and an extracellular domain of an IL4R polypeptide. Methods of producing or purifying contiguous polypeptides are also provided. Methods of treatment using contiguous polypeptides to bind IL13 and/or IL4 and inhibit IL13- and/or IL-4-mediated signaling are provided. Such methods include, but are not limited to, methods of treating IL13- and/or IL4-induced conditions in companion animal species. Methods of detecting IL13 and/or IL4 in a sample from a companion animal species are also provided.

IL13R/IL4R heterodimeric proteins that bind canine IL13 and/or IL4, feline IL13 and/or IL4, and/or equine IL13 and/or IL4 are also provided, for example long-acting proteins. In some embodiments, the IL13R/IL4R heterodimeric protein comprises a first contiguous polypeptide comprising an extracellular domain of an IL13R polypeptide and an Fc polypeptide and a second contiguous polypeptide comprising an extracellular domain of an IL4R polypeptide and an Fc polypeptide. Methods of producing or purifying IL13R/IL4R heterodimeric proteins and contiguous polypeptides are also provided. Methods of treatment using IL13R/IL4R heterodimeric proteins to bind IL13 and/or IL4 and inhibit IL13- and/or IL-4-mediated signaling are provided. Such methods include, but are not limited to, methods of treating IL13- and/or IL4-induced conditions in companion animal species. Methods of detecting IL13 and/or IL4 in a sample from a companion animal species are also provided.

Also provided are variant IgG Fc polypeptides from companion animals having increased binding to Protein A, decreased binding to C1q, decreased binding to CD16, increased binding to FcRn that may be used in the context of the contiguous polypeptides or heterodimeric proteins provided herein.

For the convenience of the reader, the following definitions of terms used herein are provided.

As used herein, numerical terms such as Kd are calculated based upon scientific measurements and, thus, are subject to appropriate measurement error. In some instances, a numerical term may include numerical values that are rounded to the nearest significant figure.

As used herein, “a” or “an” means “at least one” or “one or more” unless otherwise specified. As used herein, the term “or” means “and/or” unless specified otherwise. In the context of a multiple dependent claim, the use of “or” when referring back to other claims refers to those claims in the alternative only.

### Exemplary IL13R/IL4R Contiguous Polypeptides and Heterodimeric Proteins

Novel IL13R/IL4R contiguous polypeptides and IL13R/IL4R heterodimeric proteins are provided, for example, heterodimeric proteins that bind canine IL13 and/or IL4, feline IL13 and/or IL4, and/or equine IL13 and/or IL4.

“Amino acid sequence,” means a sequence of amino acids residues in a peptide or protein. The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

“Glycosylated,” as used herein, refers to a polypeptide having one or more glycan moieties covalently attached.

A “glycan” or “glycan moiety,” as used herein, refers to monosaccharides linked glycosidically.

Glycans are attached to glycopeptides in several ways, of which N-linked to asparagine and O-linked to serine and threonine are the most relevant for recombinant therapeutic glycoproteins. N-linked glycosylation occurs at the consensus sequence Asn-Xaa-Ser/Thr, where Xaa can be any amino acid except proline.

“Sialylated,” as used herein, refers to a polypeptide having one or more sialyic acid moieties covalently attached.

A variety of approaches for producing glycosylated and sialylated proteins have been developed. See. e.g., Savinova, et al., Applied Biochem & Microbiol. 51(8):827-33 (2015).

“PEGylated,” as used herein, refers to a polypeptide having one or more polyethylene glycol (PEG) moieties associated or covalently or non-covalently attached.

In some embodiments, a polypeptide is glycosylated. In some embodiments, a polypeptide comprises at least one glycan moiety attached to an N-linked glycosylation site. In some embodiments, a polypeptide is sialylated. In some embodiments, a polypeptide is PEGylated. In some embodiments, a polypeptide is PEGylated at a glycan. In some embodiments, a polypeptide is PEGylated at a primary amine. In some embodiments, a polypeptide is PEGylated at the N-terminal alpha-amine. In some embodiments, a polypeptide is glycosylated, sialylated, and/or PEGylated.

The term “contiguous polypeptide” herein is used to mean an uninterrupted sequence of amino acids. A contiguous polypeptide is typically translated from a single continuous DNA sequence. It can be made by genetic engineering, for example, by removing the stop codon from the DNA sequence of the first protein, then appending the DNA sequence of the second protein in frame, so that the DNA sequence is expressed as a single protein. Typically, this is accomplished by cloning a cDNA into an expression vector in frame with an existing gene.

“IL4R,” as used herein, is a polypeptide comprising the entirety or a fragment of IL4 receptor subunit alpha that binds to IL-4.

For example, “IL4R” refers to an IL4R polypeptide from any vertebrate source, including mammals such as primates (e.g., humans and cynomolgus monkeys), rodents (e.g., mice and rats), and companion animals (e.g., dogs, cats, and equine), unless otherwise indicated. In some embodiments, IL4R is an extracellular domain fragment that binds IL4. In some such embodiments, the IL4R may be referred to as an IL4R extracellular domain (ECD). In some embodiments, IL4R comprises the amino acid sequence of SEQ ID NO: 7, SEQ ID NO:8, SEQ ID NO: 9, SEQ ID NO: 23, SEQ ID NO: 163, SEQ ID NO: 25, SEQ ID NO: 27, SEQ ID NO: 33, SEQ ID NO: 35, or SEQ ID NO: 37.

“IL13R,” as used herein, is a polypeptide comprising the entirety or a portion of IL13 receptor subunit alpha-1 that binds to IL-13.

For example, “IL13R” refers to an IL13R polypeptide from any vertebrate source, including mammals such as primates (e.g., humans and cynomolgus monkeys), rodents (e.g., mice and rats), and companion animals (e.g., dogs, cats, and equine), unless otherwise indicated. In some embodiments, IL13R is an extracellular domain fragment that binds IL13. In some such embodiments, the IL13R may be referred to as an IL13R extracellular domain (ECD). In some embodiments, the IL13R polypeptide comprises the amino acid sequence of SEQ ID NO: 10, SEQ ID NO: 11, SEQ ID NO: 12, SEQ ID NO: 22, SEQ ID NO: 24, SEQ ID NO: 26, SEQ ID NO: 32, SEQ ID NO: 34, or SEQ ID NO: 36.

“IL13R decoy” or “IL13Rd” as used herein, is a polypeptide comprising the entirety or a portion of IL13 receptor subunit alpha-2 that binds IL-13.

For example, “IL13R decoy” or “IL13Rd” refers to an IL13 receptor subunit alpha-2 polypeptide from any vertebrate source, including mammals such as primates (e.g., humans and cynomolgus monkeys), rodents (e.g., mice and rats), and companion animals (e.g., dogs, cats, and equine), unless otherwise indicated. In some embodiments, IL13R decoy is an extracellular domain fragment of IL13 receptor subunit alpha-2 that binds IL13. In some such embodiments, the IL13R decoy may be referred to as an IL13R decoy extracellular domain (ECD). In some embodiments, IL13R decoy comprises the amino acid sequence of SEQ ID NO: 164, SEQ ID NO: 165, SEQ ID NO: 166, SEQ ID NO: 167, SEQ ID NO: 168, or SEQ ID NO: 169.

The term “companion animal species” refers to an animal suitable to be a companion to humans. In some embodiments, a companion animal species is a small mammal, such as a canine, feline, dog, cat, horse, rabbit, ferret, guinea pig, rodent, etc. In some embodiments, a companion animal species is a farm animal, such as a horse, cow, pig, etc.

An “extracellular domain” (“ECD”) is the portion of a polypeptide that extends beyond the transmembrane domain into the extracellular space. The term “extracellular domain,” as used herein, may comprise a complete extracellular domain or may comprise a truncated extracellular domain missing one or more amino acids, that binds to its ligand. The composition of the extracellular domain may depend on the algorithm used to determine which amino acids are in the membrane. Different algorithms may predict, and different systems may express, different extracellular domains for a given protein.

An extracellular domain of an IL4R polypeptide may comprise a complete extracellular domain or a truncated extracellular domain of IL4R that binds IL4. As used herein, the terms “extracellular domain of an IL4R polypeptide,” “IL4R ECD,” and similar terms refer to an IL4R polypeptide that does not comprise a transmembrane domain or cytoplasmic domain, even if the term follows an open transitional word, such as “comprising,” “comprises,” and the like. In some embodiments, an extracellular domain of an IL4R polypeptide is an extracellular domain of an IL4R polypeptide from a companion species animal. For example, in some embodiments, an extracellular domain of an IL4R polypeptide is from canine IL4R, feline IL4R or equine IL4R. In some embodiments, an extracellular domain of an IL4R polypeptide comprises the amino acid sequence of SEQ ID NO: 23, SEQ ID NO: 163, SEQ ID NO: 25, or SEQ ID NO: 27, or any fragment thereof. In some embodiments, an extracellular domain of an IL4R polypeptide comprises the amino acid sequence of SEQ ID NO: 33, SEQ ID NO: 35, or SEQ ID NO: 37, or any fragment thereof.

An extracellular domain of an IL13R polypeptide may comprise a complete extracellular domain or a truncated extracellular domain of IL13R that binds IL13. As used herein, the terms “extracellular domain of an IL13R polypeptide,” “IL13R ECD,” and similar terms refer to an IL13R polypeptide that does not comprise a transmembrane domain or cytoplasmic domain, even if the term follows an open transitional word, such as “comprising,” “comprises,” and the like. In some embodiments, an extracellular domain of an IL13R polypeptide is an extracellular domain of an IL13R polypeptide from a companion species animal. For example, in some embodiments, an extracellular domain of an IL13R polypeptide is from canine IL13R, feline IL13R or equine IL13R. In some embodiments, an extracellular domain of an IL13R polypeptide comprises the amino acid sequence of SEQ ID NO: 22, SEQ ID NO: 24, or SEQ ID NO: 26, or any fragment thereof. In some embodiments, an extracellular domain of an IL13R polypeptide comprises the amino acid sequence of SEQ ID NO: 32, SEQ ID NO: 34, or SEQ ID NO: 36, or any fragment thereof.

An extracellular domain of an IL13Rd polypeptide may comprise a complete extracellular domain or a truncated extracellular domain of IL13Rd that binds IL13. As used herein, the terms “extracellular domain of an IL13Rd polypeptide,” “IL13Rd ECD,” and similar terms refer to an IL13Rd polypeptide that does not comprise a transmembrane domain or cytoplasmic domain, even if the term follows an open transitional word, such as “comprising,” “comprises,” and the like. In some embodiments, an extracellular domain of an IL13Rd polypeptide is an extracellular domain of an IL13Rd polypeptide from a companion species animal. For example, in some embodiments, an extracellular domain of an IL13Rd polypeptide is from canine IL13Rd, feline IL13Rd, or equine IL13Rd. In some embodiments, an extracellular domain of an IL13Rd polypeptide comprises the amino acid sequence of SEQ ID NO: 167, SEQ ID NO: 168, or SEQ ID NO: 169, or any fragment thereof.

The terms “IL13R/IL4R contiguous polypeptide” and “IL4R/IL13R contiguous polypeptide” are used interchangeably to refer to a contiguous polypeptide comprising an IL13R polypeptide and an IL4R polypeptide, where the terms are not indicative of the order in which the IL13R and IL4R polypeptides appear in the contiguous polypeptide, unless the order is otherwise indicated. For example, an IL13R/IL4R contiguous polypeptide or an IL4R/IL13R contiguous polypeptide may refer to an IL4R polypeptide preceded in sequence or followed in sequence by an IL13R polypeptide. In addition, an IL13R/IL4R contiguous polypeptide or an IL4R/IL13R contiguous polypeptide may refer to an IL13R polypeptide preceded in sequence or followed in sequence by an IL4R polypeptide.

The terms “IL13Rd/IL4R contiguous polypeptide” and “IL4R/IL13Rd contiguous polypeptide” are used interchangeably to refer to a contiguous polypeptide comprising an IL13Rd polypeptide and an IL4R polypeptide, where the terms are not indicative of the order in which the IL13Rd and IL4R polypeptides appear in the contiguous polypeptide, unless the order is otherwise indicated. For example, an IL13Rd/IL4R contiguous polypeptide or an IL4R/IL13Rd contiguous polypeptide may refer to an IL4R polypeptide preceded in sequence or followed in sequence by an IL13Rd polypeptide. In addition, an IL13Rd/IL4R contiguous polypeptide or an IL4R/IL13Rd contiguous polypeptide may refer to an IL13Rd polypeptide preceded in sequence or followed in sequence by an IL4R polypeptide.

In some embodiments, the IL13R/IL4R contiguous polypeptide comprises an IL13R polypeptide joined to a IL4R polypeptide at the C-terminus of the IL13R polypeptide or at the N-terminus of the IL13R polypeptide. In some embodiments, the IL13R/IL4R contiguous polypeptide comprises an IL4R polypeptide joined to a IL13R polypeptide at the C-terminus of the IL4R polypeptide or at the N-terminus of the IL4R polypeptide.

In some embodiments, the IL13Rd/IL4R contiguous polypeptide comprises an IL13Rd polypeptide joined to a IL4R polypeptide at the C-terminus of the IL13Rd polypeptide or at the N-terminus of the IL13R polypeptide. In some embodiments, the IL13Rd/IL4R contiguous polypeptide comprises an IL4R polypeptide joined to a IL13Rd polypeptide at the C-terminus of the IL4R polypeptide or at the N-terminus of the IL4R polypeptide.

The IL13R/IL4R contiguous polypeptide of the invention may comprise an extracellular domain of a IL13R polypeptide and/or an extracellular domain of a IL4R polypeptide, wherein the polypeptides are from a companion animal species. For example, a contiguous polypeptide may comprise an extracellular domain of an IL4R polypeptide from a dog, cat, or horse and/or may comprise an extracellular domain of an IL13R polypeptide from a dog, cat, or horse.

The IL13Rd/IL4R contiguous polypeptide of the invention may comprise an extracellular domain of a IL13Rd polypeptide and/or an extracellular domain of a IL4R polypeptide, wherein the polypeptides are from a companion animal species. For example, a contiguous polypeptide may comprise an extracellular domain of an IL4R polypeptide from a dog, cat, or horse and/or may comprise an extracellular domain of an IL13Rd polypeptide from a dog, cat, or horse.

The terms “IL13R/IL4R heterodimeric protein” and “IL4R/IL13R heterodimeric protein” are used interchangeably to refer to a heterodimeric protein comprising a first contiguous polypeptide comprising an IL13R polypeptide and a second contiguous polypeptide comprising an IL4R polypeptide.

The terms “IL13Rd/IL4R heterodimeric protein” and “IL4R/IL13Rd heterodimeric protein” are used interchangeably to refer to a heterodimeric protein comprising a first contiguous polypeptide comprising an IL13Rd polypeptide and a second contiguous polypeptide comprising an IL4R polypeptide.

In some embodiments, the first contiguous polypeptide and/or second contiguous polypeptide comprises an Fc polypeptide.

The IL13R/IL4R heterodimeric protein of the invention may comprise an extracellular domain of a IL13R polypeptide and/or an extracellular domain of a IL4R polypeptide, wherein the polypeptides are from a companion animal species. For example, a heterodimeric protein may comprise an extracellular domain of an IL4R polypeptide from a dog, cat, or horse and/or may comprise an extracellular domain of an IL13R polypeptide from a dog, cat, or horse.

The IL13Rd/IL4R heterodimeric protein of the invention may comprise an extracellular domain of a IL13Rd polypeptide and/or an extracellular domain of a IL4R polypeptide, wherein the polypeptides are from a companion animal species. For example, a heterodimeric protein may comprise an extracellular domain of an IL4R polypeptide from a dog, cat, or horse and/or may comprise an extracellular domain of an IL13Rd polypeptide from a dog, cat, or horse.

“Wild-type” refers to a non-mutated version of a polypeptide that occurs in nature, or a fragment thereof. A wild-type polypeptide may be produced recombinantly. A “wildtype IL13R ECD,” “wildtype IL13Rd ECD,” or a “wildtype IL4R ECD” refers to a protein having an amino acid sequence that is identical to the same portion of an extracellular domain of an IL13R, IL13Rd. or IL4R that occurs in nature.

A “variant” is a nucleic acid molecule or polypeptide that differs from a reference nucleic acid molecule or polypeptide by single or multiple amino acid substitutions, deletions, and/or additions and substantially retains at least one biological activity of the reference nucleic acid molecule or polypeptide.

A “biologically active” entity, or an entity having “biological activity,” is an entity having any function related to or associated with a metabolic or physiological process, and/or having structural, regulatory, or biochemical functions of a naturally-occurring molecule. Biologically active polynucleotide fragments are those exhibiting similar activity, but not necessarily identical, to an activity of a polynucleotide of the present invention. A biologically active polypeptide or fragment thereof includes one that can participate in a biological reaction, including, but not limited to, a ligand-receptor interaction or antigen-antibody binding. The biological activity can include an improved desired activity, or a decreased undesirable activity. An entity may demonstrate biological activity when it participates in a molecular interaction with another molecule, such as hybridization, when it has therapeutic value in alleviating a disease condition, when it has prophylactic value in inducing an immune response, when it has diagnostic and/or prognostic value in determining the presence of a molecule, such as a biologically active fragment of a polynucleotide that may be detected as unique for the polynucleotide molecule, and when it can be used as a primer in a polymerase chain reaction (PCR).

As used herein, “percent (%) amino acid sequence identity” and “homology” with respect to a nucleic acid molecule or polypeptide sequence are defined as the percentage of nucleotide or amino acid residues in a reference sequence that are identical with the nucleotide or amino acid residues in the specific nucleic acid molecule or polypeptide sequence, after aligning the sequences and introducing gaps, if necessary to achieve the maximum percent sequence identity, and not considering any conservative substitutions as part of the sequence identity. Alignment for purposes of determining percent sequence identity can be achieved in various ways that are within the skill in the art, for instance, using publicly available computer software such as BLAST, BLAST-2, ALIGN, or MEGALINE™ (DNASTAR) software. Those skilled in the art can determine appropriate parameters for measuring alignment, including any algorithms needed to achieve maximal alignment over the full length of sequences being compared.

In some embodiments, a variant has at least about 50% sequence identity with the reference nucleic acid molecule or polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, deleted, at the N- or C-terminus of the polypeptide. In some embodiments, a variant has at least about 50% sequence identity, at least about 60% sequence identity, at least about 65% sequence identity, at least about 70% sequence identity, at least about 75% sequence identity, at least about 80% sequence identity, at least about 85% sequence identity, at least about 90% sequence identity, at least about 95% sequence identity, at least about 98%, or at least about 99% sequence identity with the sequence of the reference nucleic acid or polypeptide.

In some embodiments, a contiguous polypeptide comprises an extracellular domain of an IL13R polypeptide having at least 85%, at least 90%, at least 95%, at least 98%, or at least about 99% sequence identity to the amino acid sequence of SEQ ID NO: 22, SEQ ID NO: 24, SEQ ID NO: 26, SEQ ID NO: 32, SEQ ID NO: 34, or SEQ ID NO: 36. In some embodiments, a contiguous polypeptide comprises an extracellular domain of an IL13Rd polypeptide having at least 85%, at least 90%, at least 95%, at least 98%, or at least 99% sequence identity to the amino acid sequence of SEQ ID NO: 167, SEQ ID NO: 168, or SEQ ID NO: 169. In some embodiments, a contiguous polypeptide comprises an extracellular domain of an IL4R polypeptide having at least 85%, at least 90%, at least 95%, at least 98%, or at least 99% sequence identity to the amino acid sequence of SEQ ID NO: 23, SEQ ID NO: 163, SEQ ID NO: 25, SEQ ID NO: 27, SEQ ID NO: 33, SEQ ID NO: 35, or SEQ ID NO: 37.

As used herein, “position corresponding to position n,” wherein n is any number, refers to an amino acid position of a subject polypeptide that aligns with position n of a reference polypeptide after aligning the amino acid sequences of the subject and reference polypeptides and introducing gaps. Alignment for purposes of whether a position of a subject polypeptide corresponds with position n of a reference polypeptide can be achieved in various ways that are within the skill in the art, for instance, using publicly available computer software such as BLAST, BLAST-2, CLUSTAL OMEGA, ALIGN, or MEGALIGN™ (DNASTAR) software. Those skilled in the art can determine appropriate parameters for alignment, including any parameters needed to achieve maximal alignment over the full length of two sequences being compared. In some embodiments, the subject polypeptide and the reference polypeptide are of different lengths.

In some embodiments, the contiguous polypeptide comprises an extracellular domain of an IL13R polypeptide comprising a cysteine at a position corresponding to position 18 of SEQ ID NO: 22, at a position corresponding to position 18 of SEQ ID NO: 24, or at a position corresponding to position 18 of SEQ ID NO: 26. In some embodiments, the contiguous polypeptide comprises an extracellular domain of an IL13R polypeptide comprising a cysteine at position 18 of SEQ ID NO: 22, at position 18 of SEQ ID NO: 24, at position 18 of SEQ ID NO: 26, at position 15 of SEQ ID NO: 32, at position 15 of SEQ ID NO: 34, or at position 15 of SEQ ID NO: 36.

A “point mutation” is a mutation that involves a single nucleotide or amino acid residue. The mutation may be the loss of a nucleotide or amino acid, substitution of one nucleotide or amino acid residue for another, or the insertion of an additional nucleotide or amino acid residue.

An amino acid substitution may include but is not limited to the replacement of one amino acid in a polypeptide with another amino acid. Exemplary substitutions are shown in Table 2. Amino acid substitutions may be introduced into a molecule of interest and the products screened for a desired activity, for example, retained/improved antigen binding, decreased immunogenicity, or improved ADCC or CDC or enhanced pharmacokinetics.

TABLE 2

| Original | |
|---|---|
| Residue | Exemplary Substitutions |
| Ala (A) | Val; Leu; Ile |
| Arg (R) | Lys; Gln; Asn |
| Asn (N) | Gln; His; Asp; Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes with another class.

A “fusion partner,” as used herein, refers to an additional component of an IL13R/IL4R contiguous polypeptide, such as an additional polypeptide, such as albumin, an albumin binding fragment, or a fragment of an immunoglobulin molecule. A fusion partner may comprise an oligomerization domain such as an Fc domain of a heavy chain immunoglobulin.

The term “IgX Fc” or “IgX Fc polypeptide” means the Fc region is from a particular antibody isotype (e.g., IgG, IgA, IgD, IgE, IgM, etc.), where “X” denotes the antibody isotype. Thus, “IgG” or “IgG Fc” denotes the Fc region of a γ chain, “IgA” or “IgA Fc” denotes the Fc region of an α chain, “IgD” or “IgD Fc” denotes the Fc region of a δ chain, “IgE” or “IgE Fc” denotes the Fc region of an ε chain, “IgM” or “IgM Fc” denotes the Fc region of a μ chain, etc.

A “fragment crystallizable polypeptide” or “Fc polypeptide” is the portion of an antibody molecule that interacts with effector molecules and cells. It comprises the C-terminal portions of the immunoglobulin heavy chains. As used herein, an Fc polypeptide includes fragments of the Fc domain having one or more biological activities of an entire Fc polypeptide. In some embodiments, a biological activity of an Fc polypeptide is the ability to bind FcRn. In some embodiments, a biological activity of an Fc polypeptide is the ability to bind C1q. In some embodiments, a biological activity of an Fc polypeptide is the ability to bind CD16. In some embodiments, a biological activity of an Fc polypeptide is the ability to bind Protein A. An “effector function” of the Fc polypeptide is an action or activity performed in whole or in part by any antibody in response to a stimulus and may include complement fixation and/or ADCC (antibody-dependent cellular cytotoxicity) induction.

“IgX Fc” or “IgX Fc polypeptide” refers to an Fc polypeptide derived from a particular antibody isotype (e.g., IgG, IgA, IgD, IgE, IgM, etc.), where “X” denotes the antibody isotype. Thus, “IgG Fc” denotes that the Fc polypeptide is derived from a γ chain, “IgA Fc” denotes that the Fc polypeptide is derived from an α chain, “IgD Fc” denotes that the Fc polypeptide is derived from a δ chain, “IgE Fc” denotes that the Fc polypeptide is derived from a ε chain, “IgM Fc” denotes that the Fc polypeptide is derived from a μ chain, etc. In some embodiments, the IgG Fc polypeptide comprises the hinge, CH2, and CH3, but does not comprise CH1 or CL. In some embodiments, the IgG Fc polypeptide comprises CH2 and CH3, but does not comprise CH1, the hinge, or CL. In some embodiments, the IgG Fc polypeptide comprises CH1, hinge, CH2, CH3, with or without CL. In some embodiments, the IgG Fc polypeptide comprises CH1, hinge, CH2, and CH3, with or without CL1. In some embodiments, an Fc polypeptide, such as an IgG Fc polypeptide, lacks one or more C-terminal amino acids, such as 1 to 20, 1 to 15, 1 to 10, 1 to 5, or 1 to 2 amino acids, while retaining biological activity. In some embodiments, the biological activity is the ability to bind FcRn. An “effector function” of the Fc polypeptide is an action or activity performed in whole or in part by any antibody in response to a stimulus and may include complement fixation and/or ADCC (antibody-dependent cellular cytotoxicity) induction. “IgX-N Fc” or “IgGXN Fc” denotes that the Fc polypeptide is derived from a particular subclass of antibody isotype (such as canine IgG subclass IgG-A, IgG-B, IgG-C, or IgG-D; feline IgG subclass IgG1a, IgG1b, or IgG2; or equine IgG subclass IgG1, IgG2, IgG3, IgG4, IgG5, IgG6, or IgG7, etc.), where “N” denotes the subclass.

In some embodiments, IgX or IgXN regions are from a companion animal, such as a dog, a cat, or a horse. In some embodiments, IgG regions are isolated from canine γ heavy chains, such as IgGA, IgGB, IgGC, or IgGD. In some instances, IgG Fc regions are isolated from feline γ heavy chains, such as IgG1a, IgG1b, or IgG2. In other instances, IgG regions are isolated from equine γ heavy chains, such as IgG1, IgG2, IgG3, IgG4, IgG5, IgG6, or IgG7. Polypeptides comprising an Fc region of IgGA, IgGB, IgGC, or IgGD may provide for higher expression levels in recombination production systems.

In some embodiments, a contiguous polypeptide comprises a first variant IgG Fc polypeptide comprising a “knob” mutation and a second variant IgG Fc polypeptide comprising a “hole” mutation. Nonlimiting exemplary knob and hole mutations are described, for example, in Merchant, A. M. et al. An efficient route to human bispecific IgG. Nat Biotechnol, 16(7):677-81 (1998).

A “knob” mutation,” as used herein, refers to an interfacing mutation of a molecule (e.g., an Fc polypeptide) that comprises a bulky amino acid.

A “hole mutation,” as used herein, refers to an interfacing mutation of a molecule (e.g., an Fc polypeptide) that comprises one or more smaller amino acids.

In some embodiments, a variant IgG Fc polypeptide comprises a knob mutation. In some embodiments, a variant IgG Fc polypeptide comprises an amino acid substitution at a position corresponding to position 138 of SEQ ID NO: 38; position 137 of SEQ ID NO: 39, position 137 of SEQ ID NO: 40; position 138 of SEQ ID NO: 41; position 154 of SEQ ID NO: 42, SEQ ID NO: 43, SEQ ID NO: 44, SEQ ID NO: 45, or SEQ ID NO: 46; or position 130 of SEQ ID NO: 47, SEQ ID NO: 48, SEQ ID NO: 49, SEQ ID NO: 50, SEQ ID NO: 51, SEQ ID NO: 52, or SEQ ID NO: 53.

In some embodiments, a variant IgG Fc polypeptide comprises an amino acid substitution at position 138 of SEQ ID NO: 38; position 137 of SEQ ID NO: 39; position 137 of SEQ ID NO: 40; position 138 of SEQ ID NO: 41; position 154 of SEQ ID NO: 42, SEQ ID NO: 43, SEQ ID NO: 44, SEQ ID NO: 45, or SEQ ID NO: 46; or position 130 of SEQ ID NO: 47, SEQ ID NO: 48, SEQ ID NO: 49, SEQ ID NO: 50, SEQ ID NO: 51, SEQ ID NO: 52, or SEQ ID NO: 53.

In some embodiments, a variant IgG Fc polypeptide comprises a tryptophan at a position corresponding to position 138 of SEQ ID NO: 38; position 137 of SEQ ID NO: 39; position 137 of SEQ ID NO: 40; position 138 of SEQ ID NO: 41, or position 154 of SEQ ID NO: 42, SEQ ID NO: 43, SEQ ID NO: 44, SEQ ID NO: 45, or SEQ ID NO: 46; or position 130 of SEQ ID NO: 47, SEQ ID NO: 48, SEQ ID NO: 49, SEQ ID NO: 50, SEQ ID NO: 51, SEQ ID NO: 52, or SEQ ID NO: 53.

In some embodiments, a variant IgG Fc polypeptide comprises a tryptophan at position 138 of SEQ ID NO: 38; position 137 of SEQ ID NO: 39; position 137 of SEQ ID NO: 40; position 138 of SEQ ID NO: 41; position 154 of SEQ ID NO: 42, SEQ ID NO: 43, SEQ ID NO: 44, SEQ ID NO: 45, or SEQ ID NO: 46; or position 130 of SEQ ID NO: 47, SEQ ID NO: 48, SEQ ID NO: 49, SEQ ID NO: 50, SEQ ID NO: 51, SEQ ID NO: 52, or SEQ ID NO: 53.

In some embodiments, a variant IgG Fc polypeptide comprises the amino acid sequence of SEQ ID NO: 54, 55, 56, 57, 66, 67, 68, 69, 70, 81, 82, 83, 84, 85, 86, or 87.

In some embodiments, a variant IgG Fc polypeptide comprises a hole mutation. In some embodiments, a variant IgG Fc polypeptide comprises an amino acid substitution at a position corresponding to position 138 and/or position 140 and/or position 181 of SEQ ID NO: 38; position 137 and/or position 139 and/or position 180 of SEQ ID NO: 39; position 137 and/or position 139 and/or position 180 of SEQ ID NO: 40; position 138 and/or position 140 and/or position 181 of SEQ ID NO: 41; position 154 and/or position 156 and/or position 197 of SEQ ID NO: 42, SEQ ID NO: 43, SEQ ID NO: 44, SEQ ID NO: 45, or SEQ ID NO: 46; and/or position 130 and/or position 132 and/or position 173 of SEQ ID NO: 47, SEQ ID NO: 48, SEQ ID NO: 49, SEQ ID NO: 50, SEQ ID NO: 51, SEQ ID NO: 52, or SEQ ID NO: 53.

In some embodiments, a variant IgG Fc polypeptide comprises an amino acid substitution at position 138 and/or position 140 and/or position 181 of SEQ ID NO: 38; position 137 and/or position 139 and/or position 180 of SEQ ID NO: 39; position 137 and/or position 139 and/or position 180 of SEQ ID NO: 40; position 138 and/or position 140 and/or position 181 of SEQ ID NO: 41; position 154 and/or position 156 and/or position 197 of SEQ ID NO: 42, SEQ ID NO: 43, SEQ ID NO: 44, SEQ ID NO: 45, or SEQ ID NO: 46; or position 130 and/or position 132 and/or position 173 of SEQ ID NO: 47, SEQ ID NO: 48, SEQ ID NO: 49, SEQ ID NO: 50, SEQ ID NO: 51, SEQ ID NO: 52, or SEQ ID NO: 53.

In some embodiments, a variant IgG Fc polypeptide comprises a serine at a position corresponding to position 138 and/or an alanine at a position corresponding to position 140 and/or a threonine at a position corresponding to position 181 of SEQ ID NO: 38; a serine at a position corresponding to position 137 and/or an alanine at a position corresponding to position 139 and/or a threonine at a position corresponding to position 180 of SEQ ID NO: 39; a serine at a position corresponding to position 137 and/or an alanine at a position corresponding to position 139 and/or a threonine at a position corresponding to position 180 of SEQ ID NO: 40; a serine at a position corresponding to position 138 and/or an alanine at a position corresponding to position 140 and/or a threonine at a position corresponding to position 181 of SEQ ID NO: 41; a serine at a position corresponding to position 154 and/or an alanine at a position corresponding to position 156 and/or a threonine at a position corresponding to position 197 of SEQ ID NO: 42, SEQ ID NO: 43, SEQ ID NO: 44, SEQ ID NO: 45, or SEQ ID NO: 46; or a serine at a position corresponding to position 130 and/or an alanine at a position corresponding to position 132 and/or a threonine at a position corresponding to position 173 of SEQ ID NO: 47, SEQ ID NO: 48, SEQ ID NO: 49, SEQ ID NO: 50, SEQ ID NO: 51, SEQ ID NO: 52, or SEQ ID NO: 53.

In some embodiments, a variant IgG Fc polypeptide comprises a serine at position 138 and/or an alanine at position 140 and/or a threonine at position 181 of SEQ ID NO: 38; a serine at position 137 and/or an alanine at position 139 and/or a threonine at position 180 of SEQ ID NO: 39; a serine at position 137 and/or an alanine at position 139 and/or a threonine at position 180 of SEQ ID NO: 40; a serine at position 138 and/or an alanine at position 140 and/or a threonine at position 181 of SEQ ID NO: 41; a serine at position 154 and/or an alanine at position 156 and/or a threonine at position 197 of SEQ ID NO: 42, SEQ ID NO: 43, SEQ ID NO: 44, SEQ ID NO: 45, or SEQ ID NO: 46; or a serine at position 130 and/or an alanine at position 132 and/or a threonine at position 173 of SEQ ID NO: 47, SEQ ID NO: 48, SEQ ID NO: 49, SEQ ID NO: 50, SEQ ID NO: 51, SEQ ID NO: 52, or SEQ ID NO: 53.

In some embodiments, a variant IgG Fc polypeptide comprises the amino acid sequence of SEQ ID NO: 58, 59, 60, 61, 62, 63, 64, 65, 71, 72, 73, 74, 75, 76, 77, 78, 79, 80, 88, 89, 90, 91, 92, 93, 94, 95, 96, 97, 98, 99, 100, or 101.

In some embodiments, a variant IgG Fc polypeptide has modified neonatal receptor (FcRn) binding affinity. In some embodiments, a variant IgG Fc polypeptide has increased binding affinity to FcRn, such as at a low pH.

In some embodiments, a variant IgG Fc polypeptide binds to FcRn with an affinity greater than the wild-type IgG Fc polypeptide, as measured by biolayer interferometry, surface plasmon resonance, or any protein-protein interaction tool at a pH in the range of from about 5.0 to about 6.5, such as at a pH of about 5.0, a pH of about 5.2, a pH of about 5.5, a pH of about 6.0, a pH of about 6.2, or a pH of about 6.5.

In some embodiments, a variant IgG Fc polypeptide binds to FcRn with a dissociation constant (Kd) of less than 5×10⁻⁶ M, less than 1×10⁻⁶ M, less than 5×10⁻⁷ M, less than 1×10⁻⁷ M, less than 5×10⁻⁸ M, less than 1×10⁻⁸ M, less than 5×10⁻⁹ M, less than 1×10⁻⁹ M, less than 5×10⁻¹⁰ M, less than 1×10⁻¹⁰ M, less than 5×10⁻¹¹ M, less than 1×10⁻¹¹ M, less than 5×10⁻¹² M, or less than 1×10⁻¹² M, as measured by biolayer interferometry, surface plasmon resonance, or any protein-protein interaction tool at a pH in the range of from about 5.0 to about 6.5, such as at a pH of about 5.0, a pH of about 5.5, a pH of about 6.0, or a pH of about 6.5.

In some embodiments, a long-acting IL13R/IL4R contiguous polypeptide, IL13Rd/IL4R contiguous polypeptide, IL13Rd/IL4R heterodimeric protein, or IL13R/IL4R heterodimeric protein is provided. In some embodiments, an IL13R/IL4R contiguous polypeptide, an IL13Rd/IL4R contiguous polypeptide, an IL13Rd/IL4R heterodimeric protein, or an IL13R/IL4R heterodimeric protein has increased serum half-life. In some embodiments, the IL13R/IL4R contiguous polypeptide, IL13Rd/IL4R contiguous polypeptide, IL13Rd/IL4R heterodimeric protein, or IL13R/IL4R heterodimeric protein comprises a variant Fc polypeptide, wherein the contiguous polypeptide or heterodimeric protein has increased serum half-life relative to the contiguous polypeptide or heterodimeric protein comprising a wild-type Fc polypeptide.

In some embodiments, an IL13R/IL4R contiguous polypeptide, an IL13Rd/IL4R contiguous polypeptide, an IL13Rd/IL4R heterodimeric protein, or an IL13R/IL4R heterodimeric protein comprises a variant IgG Fc polypeptide capable of binding to FcRn with an increased affinity relative to the wild-type Fc polypeptide and wherein the contiguous polypeptide has increased serum half-life relative to a contiguous polypeptide comprising a wild-type Fc polypeptide.

In some embodiments, a contiguous polypeptide or heterodimeric protein comprises a variant IgG Fc polypeptide comprising:
a) a tyrosine or a phenylalanine at a position corresponding to position 23 of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53;
b) a tyrosine at a position corresponding to position 82 of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53;
c) a tyrosine at a position corresponding to position 82 and a histidine at a position corresponding to position 207 of SEQ ID NO: 39, SEQ ID NO: 40, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53;
d) a tyrosine at a position corresponding to position 82 and a tyrosine at a position corresponding to position 207 of SEQ ID NO: 39, SEQ ID NO: 40, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53;
e) a tyrosine at a position corresponding to position 207 of SEQ ID NO: 39, SEQ ID NO: 40, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53;
f) a tyrosine at a position corresponding to position 82 and a histidine at a position corresponding to position 208 of SEQ ID NO: 38 or SEQ ID NO: 41;
g) a tyrosine at a position corresponding to position 82 and a tyrosine at a position corresponding to position 208 of SEQ ID NO: 38 or SEQ ID NO: 41; or
h) a tyrosine at a position corresponding to position 208 of SEQ ID NO: 38 or SEQ ID NO: 41.

In some embodiments, a contiguous polypeptide or heterodimeric protein comprises a variant IgG Fc polypeptide comprising:
a) a tyrosine or a phenylalanine at position 23 of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53;
b) a tyrosine at position 82 of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53;
c) a tyrosine at position 82 and a histidine at position 207 of SEQ ID NO: 39, SEQ ID NO: 40, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53;
d) a tyrosine at position 82 and a tyrosine at position 207 SEQ ID NO: 39, SEQ ID NO: 40, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53;
e) a tyrosine at position 207 of SEQ ID NO: 39, SEQ ID NO: 40, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53;
f) a tyrosine at position 82 and a histidine at position 208 of SEQ ID NO: 38 or SEQ ID NO: 41;
g) a tyrosine at position 82 and a tyrosine at position 208 of SEQ ID NO: 38 or SEQ ID NO: 41; or
h) a tyrosine at position 208 of SEQ ID NO: 38 or SEQ ID NO: 41.

A “signal sequence” refers to a sequence of amino acid residues or polynucleotides encoding such, which facilitates secretion of a polypeptide of interest and is typically cleaved upon export of the polypeptide to the outside of the cell surface membrane.

A “linker” refers to one or more amino acid residues that connects a first polypeptide with a second polypeptide.

In some embodiments, the linker is a glycine-rich and/or serine-rich, flexible, non-structural linker. In some embodiments, a linker comprises the amino acids G (Gly) and/or S (Ser). For example, a linker may comprise G or a repeat of G (e.g., GG, GGG, etc.); GS or a repeat of GS (e.g., GSGS (SEQ ID NO: 151), GSGSGS (SEQ ID NO: 152), etc.); GGS or a repeat thereof (e.g., GGSGGS (SEQ ID NO: 153), GGSGGSGGS (SEQ ID NO: 154), etc.); GGGS (SEQ ID NO: 155) or a repeat thereof (e.g., GGGSGGGS (SEQ ID NO: 156), GGGSGGGSGGGS (SEQ ID NO: 157), etc.); GSS or a repeat thereof (e.g., GSSGSS (SEQ ID NO: 158), GSSGSSGSS (SEQ ID NO: 159), etc.); or GGSS (SEQ ID NO: 160) or a repeat thereof (e.g., GGSSGGSS (SEQ ID NO: 161), GGSSGGSSGGSS (SEQ ID NO: 162), etc.).

In some embodiments, the contiguous polypeptide comprises at least one linker. In some embodiments, the contiguous polypeptide comprises an optional signal sequence, and at least one optional linker. In some embodiments, the contiguous polypeptide does not comprise a signal sequence, or a linker. In some embodiments, the contiguous polypeptide is translated with a signal sequence, but the signal sequence is cleaved from the contiguous polypeptide.

In some embodiments, an IL13R/IL4R contiguous polypeptide comprises:
Formula (I): IL13R-L1-IL4R-L2-FP,
Formula (II): IL4R-L1-IL13R-L2-FP,
Formula (III): IL13R-L1-FP-L2-IL4R,
Formula (IV): IL4R-L1-FP-L2-IL13R,
Formula (V): FP-L1-IL13R-L2-IL4R, or
Formula (VI): FP-L1-IL4R-L2-IL13R,
   wherein IL13R is an IL13R extracellular domain (ECD) polypeptide from a companion animal species, IL4R is an IL4R ECD polypeptide from a companion animal species, L1 is a first optional linker, L2 is a second optional linker, and FP is an optional fusion partner, such as an Fc polypeptide.

In some embodiments, an IL13Rd/IL4R contiguous polypeptide comprises:
Formula (I): IL13Rd-L1-IL4R-L2-FP,
Formula (II): IL4R-L1-IL13Rd-L2-FP,
Formula (III): IL13Rd-L1-FP-L2-IL4R,
Formulat (IV): IL4R-L1-FP-L2-IL13Rd,
Formula (V): FP-L1-IL13Rd-L2-IL4R, or
Formula (VI): FP-L1-IL4R-L2-IL13Rd,
   wherein IL13Rd is an IL13Rd extracellular domain (ECD) polypeptide from a companion animal species, IL4R is an IL4R ECD polypeptide from a companion animal species, L1 is a first optional linker, L2 is a second optional linker, and FP is an optional fusion partner, such as a Fc polypeptide.

In some embodiments, an IL13R/IL4R contiguous polypeptide comprises an amino acid sequence selected from SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 28, SEQ ID NO: 29, SEQ ID NO: 30, and SEQ ID NO: 31.

In some embodiments, an IL13Rd/IL4R contiguous polypeptide comprises an amino acid sequence selected from SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, and SEQ ID NO: 183.

In some embodiments, a heterodimeric protein comprises a) a first contiguous polypeptide comprising at least one IL13R extracellular domain (ECD) and a first Fc polypeptide and b) a second contiguous polypeptide comprising at least one IL4R ECD and a second Fc polypeptide, wherein the IL13R ECD and/or the IL4R ECD are from a companion animal species.

In some embodiments, a first contiguous polypeptide or a second contiguous polypeptide has the formula:
IL13R(n)-L-Fc or
IL4R(n)-L-Fc,
   wherein IL13R(n) is at least one IL13R extracellular domain (ECD) polypeptide from a companion animal species, IL4R(n) is at least one IL4R ECD polypeptide from a companion animal species, (n) is one, two, three, four, or more ECD polypeptides, L is an optional linker, Fc is a variant Fc polypeptide, such as a variant Fc polypeptide comprising knob or a hole mutation.

In some embodiments, a heterodimeric protein comprises a) a first contiguous polypeptide comprising at least one IL13Rd extracellular domain (ECD) and a first Fc polypeptide and b) a second contiguous polypeptide comprising at least one IL4R ECD and a second Fc polypeptide, wherein the IL13Rd ECD and/or the IL4R ECD are from a companion animal species.

In some embodiments, a first contiguous polypeptide or a second contiguous polypeptide has the formula:
IL13Rd(n)-L-Fc or
IL4R(n)-L-Fc,
   wherein IL13Rd(n) is at least one IL13Rd extracellular domain (ECD) polypeptide from a companion animal species, IL4R(n) is at least one IL4R ECD polypeptide from a companion animal species, (n) is one, two, three, four, or more ECD polypeptides, L is an optional linker, Fc is a variant Fc polypeptide, such as a variant Fc polypeptide comprising knob or a hole mutation.

In addition, other binding partner(s) may be included in the contiguous polypeptide before, after, and/or between any one or more IL13R, IL13Rd, or IL4R ECD polypeptide(s). Other potential binding partners include: IL5, IL6, IL17, IL22, IL31, LFA-1, TNF-α, TSLP, and/or IgE.

In some embodiments, the heterodimeric protein comprises a first contiguous polypeptide comprising the amino acid sequence of SEQ ID NO: 103, SEQ ID NO: 105, SEQ ID NO: 107, SEQ ID NO: 109, SEQ ID NO: 111, or SEQ ID NO: 113.

In some embodiments, the heterodimeric protein comprises a second contiguous polypeptide comprising the amino acid sequence of SEQ ID NO: 102, SEQ ID NO: 104, SEQ ID NO: 106, SEQ ID NO: 108, SEQ ID NO: 110, or SEQ ID NO: 112.

In some embodiments, the contiguous polypeptide or heterodimeric protein comprises the amino acid sequence of SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, or SEQ ID NO: 183.

### Exemplary Expression and Production

Polynucleotide sequences that encode all or part (e.g., the extracellular domain) of a contiguous polypeptide with or without a signal sequence are provided. If a homologous signal sequence (i.e., a signal sequence of native IL4R, IL13R, or IL13Rd) is not used in the construction of the nucleic acid molecule, then another signal sequence may be used, for example, any one of the signal sequences described in PCT/US06/02951.

Typically, nucleotide sequence encoding the polypeptide of interest, such as a contiguous polypeptide, is inserted into an expression vector, suitable for expression in a selected host cell.

A “vector” is a plasmid that can be used to transfer DNA sequences from one organism to another or to express a gene of interest. A vector typically includes an origin of replication and regulatory sequences which regulate the expression of the gene of interest, and may or may not carry a selective marker gene, such as an antibiotic resistance gene. A vector is suitable for the host cell in which it is to be expressed. A vector may be termed a “recombinant vector” when the gene of interest is present in the vector.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), 293 cells, and CHO cells, and their derivatives, such as 293-6E, DG44, CHO-S, and CHO-K cells. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) encoding an amino acid sequence(s) provided herein.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated.”

In some embodiments, the heterodimeric protein or contiguous polypeptide is isolated using chromatography, such as size exclusion chromatography, ion exchange chromatography, protein A column chromatography, hydrophobic interaction chromatography, and CHT chromatography.

The terms “label” and “detectable label” mean a moiety attached to a IL13R/IL4R contiguous polypeptide to render it detectable. In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels for polypeptides include, but are not limited to, the following: radioisotopes or radionuclides (for example, ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); chromogens, fluorescent labels (for example, FITC, rhodamine, lanthanide phosphors), enzymatic labels (for example, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In this regard, the moiety itself may not be detectably labeled but may become detectable upon reaction with yet another moiety.

### Exemplary IL13R/IL4R Contiguous Polypeptides, IL13Rd/IL4R Contiguous Polypeptides, IL13R/IL4R Heterodimeric Proteins, and IL13Rd/IL4R Heterodimeric Proteins as Decoy Receptor Traps

The IL13R/IL4R contiguous polypeptides, IL13Rd/IL4R contiguous polypeptides, IL13R/IL4R heterodimeric proteins, and IL13Rd/IL4R heterodimeric proteins of the invention can function as decoy receptors for trapping IL13 and/or IL4 and inhibiting their interaction with IL13R and/or IL4R on cell surfaces. Decoy receptors, such as those of the invention, recognize their ligands with high affinity and specificity but are structurally incapable of signaling. They compete with wild-type receptors for ligand binding and participate in ligand/receptor interactions, thus modulating the activity of or the number of functioning receptors and/or the cellular activity downstream from the receptors. Decoy receptors can act as molecular traps for agonist ligands and thereby inhibit ligand-induced receptor activation.

“IL13” as used herein refers to any native IL13 that results from expression and processing of IL13 in a cell. The term includes IL13 from any vertebrate source, including mammals such as primates (e.g., humans and cynomolgus monkeys) and rodents (e.g., mice and rats), and companion animals (e.g., dogs, cats, and equine), unless otherwise indicated. The term also includes naturally occurring variants of IL13, e.g., splice variants or allelic variants.

In some embodiments, a canine IL13 comprises the amino acid sequence of SEQ ID NO: 4. In some embodiments, a feline IL13 comprises the amino acid sequence of SEQ ID NO: 5. In some embodiments, an equine IL13 comprises the amino acid sequence of SEQ ID NO: 6.

“IL4” as used herein refers to any native IL4 that results from expression and processing of IL4 in a cell. The term includes IL4 from any vertebrate source, including mammals such as primates (e.g., humans and cynomolgus monkeys) and rodents (e.g., mice and rats), and companion animals (e.g., dogs, cats, and equine), unless otherwise indicated. The term also includes naturally occurring variants of IL4, e.g., splice variants or allelic variants.

In some embodiments, a canine IL4 comprises the amino acid sequence of SEQ ID NO: 1. In some embodiments, a feline IL4 comprises the amino acid sequence of SEQ ID NO: 2. In some embodiments, an equine IL4 comprises the amino acid sequence of SEQ ID NO: 3.

The invention provides IL13R/IL4R contiguous polypeptides, IL13Rd/IL4R contiguous polypeptides, IL13R/IL4R heterodimeric proteins, and IL13Rd/IL4R heterodimeric proteins as therapeutic agents. The contiguous and heterodimeric proteins of the invention bind to IL13 and/or IL4, described in more detail herein, which have been demonstrated to be associated with allergic diseases. In various embodiments, the contiguous and heterodimeric proteins of the invention can bind IL13 and/or IL4 with very high affinity. In various embodiments, the contiguous and heterodimeric proteins of the invention can interfere with IL13 and/or IL4 signaling.

The term “affinity” means the strength of the sum total of noncovalent interactions between a single binding site of a molecule (for example, a receptor) and its binding partner (for example, a ligand). The affinity of a molecule X for its partner Y can generally be represented by the dissociation constant (K_{D}). Affinity can be measured by common methods known in the art, such as, for example, immunoblot, ELISA KD, KinEx A, biolayer interferometry (BLI), or surface plasmon resonance devices.

The terms “K_{D},” “K_{d},” “Kd” or “Kd value” as used interchangeably to refer to the equilibrium dissociation constant of a receptor fusion-ligand interaction. In some embodiments, the K_{d} of the fusion molecule to its ligand is measured by using biolayer interferometry assays using a biosensor, such as an Octet® System (Pall ForteBio LLC, Fremont, CA) according to the supplier's instructions. Briefly, biotinylated antigen is bound to the sensor tip and the association of fusion molecule is monitored for ninety seconds and the dissociation is monitored for 600 seconds. The buffer for dilutions and binding steps is 20 mM phosphate, 150 mM NaCl, pH 7.2. A buffer only blank curve is subtracted to correct for any drift. The data are fit to a 2:1 binding model using ForteBio data analysis software to determine association rate constant (kₒₙ), dissociation rate constant (k_{off}), and the K_{d}. The equilibrium dissociation constant (K_{d}) is calculated as the ratio of k_{off}/kₒₙ. The term “kₒₙ” refers to the rate constant for association of a molecule X to its partner Y and the term “k_{off}” refers to the rate constant for dissociation of a molecule X or partner Y from the molecule X/partner Y complex.

The term “binds” to a substance is a term that is well understood in the art, and methods to determine such binding are also well known in the art. A molecule is said to exhibit “binding” if it reacts, associates with, or has affinity for a particular cell or substance and the reaction, association, or affinity is detectable by one or more methods known in the art, such as, for example, immunoblot, ELISA KD, KinEx A, biolayer interferometry (BLI), surface plasmon resonance devices, or etc.

“Surface plasmon resonance” denotes an optical phenomenon that allows for the analysis of real-time biospecific interactions by detection of alterations in protein concentrations within a biosensor matrix, for example using the BIAcore™ system (BIAcore International AB, a GE Healthcare company, Uppsala, Sweden and Piscataway, N.J.). For further descriptions, see Jonsson et al. (1993) Ann. Biol. Clin. 51: 19-26.

“Biolayer interferometry” refers to an optical analytical technique that analyzes the interference pattern of light reflected from a layer of immobilized protein on a biosensor tip and an internal reference layer. Changes in the number of molecules bound to the biosensor tip cause shifts in the interference pattern that can be measured in real-time. A nonlimiting exemplary device for biolayer interferometry is an Octet® system (Pall ForteBio LLC). See, e.g., Abdiche et al., 2008, Anal. Biochem. 377: 209-277.

In some embodiments, an IL13R/IL4R contiguous polypeptide, IL13Rd/IL4R contiguous polypeptide, an IL13R/IL4R heterodimeric protein, or an IL13Rd/IL4R heterodimeric protein binds to canine IL13 and/or IL4, feline IL13 and/or IL4, or equine IL13 and/or IL4 with a dissociation constant (Kd) of less than 5×10⁻⁶ M, less than 1×10⁻⁶ M, less than 5×10⁻⁷ M, less than 1×10⁻⁷ M, less than 5×10⁻⁸ M, less than 1×10⁻⁸ M, less than 5×10⁻⁹ M, less than 1×10⁻⁹ M, less than 5×10⁻¹⁰ M, less than 1×10⁻¹⁰ M, less than 5×10⁻¹¹ M, less than 1×10⁻¹¹ M, less than 5×10⁻¹² M, or less than 1×10⁻¹² M, as measured by biolayer interferometry. In some embodiments, an IL13R/IL4R contiguous polypeptide, IL13Rd/IL4R contiguous polypeptide, an IL13R/IL4R heterodimeric protein, or an IL13Rd/IL4R heterodimeric protein binds to canine IL13 and/or IL4, feline IL13 and/or IL4, or equine IL13 and/or IL4 with a Kd of between 5×10⁻⁶ M and 1×10⁻⁶ M, between 5×10⁻⁶ M and 5×10⁻⁷ M, between 5×10⁻⁶ M and 1×10⁻⁷ M, between 5×10⁻⁶ M and 5×10⁻⁸ M, 5×10⁻⁶ M and 1×10⁻⁸ M, between 5×10⁻⁶ M and 5×10⁻⁹ M, between 5×10⁻⁶ M and 1×10⁻⁹ M, between 5×10⁻⁶ M and 5×10⁻¹⁰ M, between 5×10⁻⁶ M and 1×10⁻¹⁰ M, between 5×10⁻⁶ M and 5×10⁻¹¹ M, between 5×10⁻⁶ M and 1×10⁻¹¹ M, between 5×10⁻⁶ M and 5×10⁻¹² M, between 5×10⁻⁶ M and 1×10⁻¹² M, between 1×10⁻⁶ M and 5×10⁻⁷ M, between 1×10⁻⁶ M and 1×10⁻⁷M, between 1×10⁻⁶ M and 5×10⁻⁸ M, 1×10⁻⁶ M and 1×10⁻⁸ M, between 1×10⁻⁶ M and 5×10⁻⁹ M, between 1×10⁻⁶ M and 1×10⁻⁹ M, between 1×10⁻⁶ M and 5×10⁻¹⁰ M, between 1×10⁻⁶ M and 1×10⁻¹⁰ M, between 1×10⁻⁶ M and 5×10⁻¹¹ M, between 1×10⁻⁶ M and 1×10⁻¹¹ M, between 1×10⁻⁶ M and 5×10⁻¹² M, between 1×10⁻⁶ M and 1×10⁻¹² M, between 5×10⁻⁷ M and 1×10⁻⁷ M, between 5×10⁻⁷ M and 5×10⁻⁸ M, 5×10⁻⁷ M and 1×10⁻⁸ M, between 5×10⁻⁷ M and 5×10⁻⁹ M, between 5×10⁻⁷ M and 1×10⁻⁹ M, between 5×10⁻⁷ M and 5×10⁻¹⁰ M, between 5×10⁻⁷ M and 1×10⁻¹⁰ M, between 5×10⁻⁷ M and 5×10⁻¹¹ M, between 5×10⁻⁷ M and 1×10⁻¹¹ M, between 5×10⁻⁷ M and 5×10⁻¹² M, between 5×10⁻⁷ M and 1×10⁻¹² M, between 1×10⁻⁷ M and 5×10⁻⁸ M, 1×10⁻⁷ M and 1×10⁻⁸ M, between 1×10⁻⁷ M and 5×10⁻⁹ M, between 1×10⁻⁷ M and 1×10⁻⁹ M, between 1×10⁻⁷ M and 5×10⁻¹⁰ M, between 1×10⁻⁷ M and 1×10⁻¹⁰ M, between 1×10⁻⁷ M and 5×10⁻¹¹ M, between 1×10⁻⁷ M and 1×10⁻¹¹ M, between 1×10⁻⁷ M and 5×10⁻¹² M, between 1×10⁻⁷ M and 1×10⁻¹² M, between 5×10⁻⁸ M and 1×10⁻⁸ M, between 5×10⁻⁸ M and 5×10⁻⁹ M, between 5×10⁻⁸ M and 1×10⁻⁹ M, between 5×10⁻⁸ M and 5×10⁻¹⁰ M, between 5×10⁻⁸ M and 1×10⁻¹⁰ M, between 5×10⁻⁸ M and 5×10⁻¹¹ M, between 5×10⁻⁸ M and 1×10⁻¹¹ M, between 5×10⁻⁸ M and 5×10⁻¹² M, between 5×10⁻⁸ M and 1×10⁻¹² M, 1×10⁻⁸ M and 5×10⁻⁹ M, between 1×10⁻⁸ M and 1×10⁻⁹ M, between 1×10⁻⁸ M and 5×10⁻¹⁰ M, between 1×10⁻⁸ M and 1×10⁻¹⁰ M, between 1×10⁻⁸ M and 5×10⁻¹¹ M, between 1×10⁻⁸ M and 1×10⁻¹¹ M, between 1×10⁻⁸ M and 5×10⁻¹² M, between 1×10⁻⁸ M and 1×10⁻¹² M, between 5×10⁻⁹ M and 1×10⁻⁹ M, between 5×10⁻⁹ M and 5×10⁻¹⁰ M, between 5×10⁻⁹ M and 1×10⁻¹⁰ M, between 5×10⁻⁹ M and 5×10⁻¹¹ M, between 5×10⁻⁹ M and 1×10⁻¹¹ M, between 5×10⁻⁹ M and 5×10⁻¹² M, between 5×10⁻⁹ M and 1×10⁻¹² M, between 1×10⁻⁹ M and 5×10⁻¹⁰ M, between 1×10⁻⁹ M and 1×10⁻¹⁰ M, between 1×10⁻⁹ M and 5×10⁻¹¹ M, between 1×10⁻⁹ M and 1×10⁻¹¹ M, between 1×10⁻⁹ M and 5×10⁻¹² M, between 1×10⁻⁹ M and 1×10⁻¹² M, between 5×10⁻¹⁰ M and 1×10⁻¹⁰ M, between 5×10⁻¹⁰ M and 5×10⁻¹¹ M, between, 1×10⁻¹⁰ M and 5×10⁻¹¹ M, 1×10⁻¹⁰ M and 1×10⁻¹¹ M, between 1×10⁻¹⁰ M and 5×10⁻¹² M, between 1×10⁻¹⁰ M and 1×10⁻¹² M, between 5×10⁻¹¹ M and 1×10⁻¹² M, between 5×10₋₁₁ M and 5×10⁻¹², between 5×10⁻¹¹ M and 1×10⁻¹¹ M, between 1×10⁻¹¹ M and 5×10⁻¹² M, or between 1×10⁻¹¹ M and 1×10⁻¹² M, as measured by biolayer interferometry. In some embodiments, an IL13R/IL4R contiguous polypeptide, an IL13Rd/IL4R contiguous polypeptide, an IL13R/IL4R heterodimeric protein, or an IL13R/IL4R heterodimeric protein binds to canine IL13 and/or IL4, feline IL13 and/or IL4, and/or equine IL13 and/or IL4.

To “reduce” or “inhibit” means to decrease, reduce, or arrest an activity, function, or amount as compared to a reference. In some embodiments, by “reduce” or “inhibit” is meant the ability to cause an overall decrease of 20% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control dose (such as a placebo) over the same period of time. A “reference” as used herein, refers to any sample, standard, or level that is used for comparison purposes. A reference may be obtained from a healthy or non-diseased sample. In some examples, a reference is obtained from a non-diseased or non-treated sample of a companion animal. In some examples, a reference is obtained from one or more healthy animals of a particular species, which are not the animal being tested or treated.

The term “substantially reduced,” as used herein, denotes a sufficiently high degree of reduction between a numeric value and a reference numeric value such that one of skill in the art would consider the difference between the two values to be of statistical significance within the context of the biological characteristic measured by said values. In some embodiments, the substantially reduced numeric values is reduced by greater than about any one of 10%, 15% 20%, 25%, 30%, 35%, 40%, 45%, 50%, 60%, 70%, 80%, 90%, or 100% compared to the reference value.

In some embodiments, an IL13R/IL4R contiguous polypeptide, an IL13Rd/IL4R contiguous polypeptide, an IL13R/IL4R heterodimeric protein, or an IL13Rd/IL4R heterodimeric protein may reduce IL13 and/or IL4 signaling in a companion animal species by at least 10%, at least 15%, at least 20%, at least 25%, at least 30%, at least 35%, at least 40%, at least 45%, at least 50%, at least 60%, at least 70%, at least 80%, at least 90%, or 100% compared to IL13 and/or IL4 signaling in the absence of the fusion molecule. In some embodiments, signaling is measured by a reduction in IL4-dependent TF-1 cell proliferation. In some embodiments, the reduction in IL13 and/or IL4 signaling or the reduction in proliferation is between 10% and 15%, between 10% and 20%, between 10% and 25%, between 10% and 30%, between 10% and 35%, between 10% and 40%, between 10% and 45%, between 10% and 50%, between 10% and 60%, between 10% and 70%, between 10% and 80%, between 10% and 90%, between 10% and 100%, between 15% and 20%, between 15% and 25%, between 15% and 30%, between 15% and 35%, between 15% and 40%, between 15% and 45%, between 15% and 50%, between 15% and 60%, between 15% and 70%, between 15% and 80%, between 15% and 90%, between 15% and 100%, between 20% and 25%, between 20% and 30%, between 20% and 35%, between 20% and 40%, between 20% and 45%, between 20% and 50%, between 20% and 60%, between 20% and 70%, between 20% and 80%, between 20% and 90%, between 20% and 100%, between 25% and 30%, between 25% and 35%, between 25% and 40%, between 25% and 45%, between 25% and 50%, between 25% and 60%, between 25% and 70%, between 25% and 80%, between 25% and 90%, between 25% and 100%, between 30% and 35%, between 30% and 40%, between 30% and 45%, between 30% and 50%, between 30% and 60%, between 30% and 70%, between 30% and 80%, between 30% and 90%, between 30% and 100%, between 35% and 40%, between 35% and 45%, between 35% and 50%, between 35% and 60%, between 35% and 70%, between 35% and 80%, between 35% and 90%, between 35% and 100%, between 40% and 45%, between 40% and 50%, between 40% and 60%, between 40% and 70%, between 40% and 80%, between 40% and 90%, between 40% and 100%, between 45% and 50%, between 45% and 60%, between 45% and 70%, between 45% and 80%, between 45% and 90%, between 45% and 100%, between 50% and 60%, between 50% and 70%, between 50% and 80%, between 50% and 90%, between 50% and 100%, between 60% and 70%, between 60% and 80%, between 60% and 90%, between 60% and 100%, between 70% and 80%, between 70% and 90%, between 70% and 100%, between 80% and 90%, between 80% and 100%, or between 90% and 100%.

“Increased” or “greater” means an increase relative to a reference. In some embodiments, by “increased” or “greater” is meant the ability to cause an overall increase of about 5% or more, of about 10% or more, of about 20% or more, of about 30% or more, of about 40% or more, of about 50% or more, of about 60% or more, of about 70% or more, of about 80% or more, of about 90% or more, of about 100% or more, of about 125% or more, of about 150% or more, of about 200% or more, or of about 300% or more relative to a reference value. In some embodiments, by “increase” or “greater” is meant the ability to cause an overall increase of about 5% to about 50%, of about 10% to about 20%, of about 50% to about 100%, of about 25% to about 70% relative to a reference value.

In some embodiments, a variant Fc polypeptide, such as a variant IgG Fc polypeptide, is capable of binding to FcRn or FcRn/B2M with an increased affinity of about 5% or more, of about 10% or more, of about 20% or more, of about 30% or more, of about 40% or more, of about 50% or more, of about 60% or more, of about 70% or more, of about 80% or more, of about 90% or more, of about 100% or more, of about 125% or more, of about 150% or more, of about 200% or more, or of about 300% or more relative to a reference Fc polypeptide. In some embodiments, a variant Fc polypeptide is capable of binding to FcRn or FcRn/B2M with an increased affinity of about 5% to about 50%, of about 10% to about 20%, of about 50% to about 100%, of about 25% to about 70% relative to a reference Fc polypeptide. In some embodiments, the reference Fc polypeptide is a wild-type Fc polypeptide. In some embodiments, the Fc polypeptide is a different variant Fc polypeptide. In some embodiments, the affinity is measured by biolayer interferometry at a pH in the range of from about 5.0 to about 6.5.

In some embodiments, a pharmacokinetic analysis is performed to determine any number of pharmacokinetic parameters including half-life, Tmax, Cmax, and Area under the Curve (AUC). For example, an animal may be administered an IL13R/IL4R contiguous polypeptide, an IL13Rd/IL4R contiguous polypeptide, an IL13R/IL4R heterodimeric protein, or an IL13Rd/IL4R heterodimeric protein described herein and serum samples collected at different time intervals (e.g., pre-injection and/or at 0.5, 1, 6, 24, 48, 72, 168, 216, and/or 336 hours post administration). The contiguous polypeptide or heterodimeric protein concentrations in the serum samples may be determined, for example by ELISA.

In some embodiments, an IL13R/IL4R contiguous polypeptide, an IL13Rd/IL4R contiguous polypeptide, an IL13R/IL4R heterodimeric protein, or an IL13Rd/IL4R heterodimeric protein has a serum half-life of about 5% or more, of about 10% or more, of about 20% or more, of about 30% or more, of about 40% or more, of about 50% or more, of about 60% or more, of about 70% or more, of about 80% or more, of about 90% or more, of about 100% or more, of about 125% or more, of about 150% or more, of about 200% or more, of about 250% or more, or of about 300% or more relative to a reference contiguous polypeptide or heterodimeric protein. In some embodiments, an IL13R/IL4R contiguous polypeptide, an IL13Rd/IL4R contiguous polypeptide, an IL13R/IL4R heterodimeric protein, or an IL13Rd/IL4R heterodimeric protein has a serum half-life of about 5% to about 50%, of about 10% to about 20%, of about 50% to about 100%, of about 25% to about 70% relative to a reference contiguous polypeptide or heterodimeric protein. In some embodiments, an IL13R/IL4R contiguous polypeptide, an IL13Rd/IL4R contiguous polypeptide, an IL13R/IL4R heterodimeric protein, or an IL13Rd/IL4R heterodimeric protein has a serum half-life of about 1.5 times or more, about 2 times or more, about 3 times or more relative to a reference contiguous polypeptide or heterodimeric protein. In some embodiments, an IL13R/IL4R contiguous polypeptide, an IL13Rd/IL4R contiguous polypeptide, an IL13R/IL4R heterodimeric protein, or an IL13Rd/IL4R heterodimeric protein has a serum half-life of about 5% to about 50%, of about 10% to about 20%, of about 50% to about 100%, of about 25% to about 70%, of about 200% to about 300% more relative to a reference contiguous polypeptide or heterodimeric protein. In some embodiments, the reference contiguous polypeptide or heterodimeric protein comprises a wild-type Fc polypeptide. In some embodiments, the Fc polypeptide is a different variant Fc polypeptide.

### Exemplary Pharmaceutical Compositions

The terms “pharmaceutical formulation” and “pharmaceutical composition” refer to a preparation which is in such form as to permit the biological activity of the active ingredient(s) to be effective, and which contains no additional components that are unacceptably toxic to a subject to which the formulation would be administered.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. Examples of pharmaceutically acceptable carriers include alumina; aluminum stearate; lecithin; serum proteins, such as human serum albumin, canine or other animal albumin; buffers such as phosphate, citrate, tromethamine or HEPES buffers; glycine; sorbic acid; potassium sorbate; partial glyceride mixtures of saturated vegetable fatty acids; water; salts or electrolytes, such as protamine sulfate, disodium hydrogen phosphate, potassium hydrogen phosphate, sodium chloride, zinc salts, colloidal silica, or magnesium trisilicate; polyvinyl pyrrolidone, cellulose-based substances; polyethylene glycol; sucrose; mannitol; or amino acids including, but not limited to, arginine.

The pharmaceutical composition can be stored in lyophilized form. Thus, in some embodiments, the preparation process includes a lyophilization step. The lyophilized composition may then be reformulated, typically as an aqueous composition suitable for parenteral administration, prior to administration to the dog, cat, or horse. In other embodiments, particularly where the fusion molecule is highly stable to thermal and oxidative denaturation, the pharmaceutical composition can be stored as a liquid, i.e., as an aqueous composition, which may be administered directly, or with appropriate dilution, to the dog, cat, or horse. A lyophilized composition can be reconstituted with sterile Water for Injection (WFI). Bacteriostatic reagents, such benzyl alcohol, may be included. Thus, the invention provides pharmaceutical compositions in solid or liquid form.

The pH of the pharmaceutical compositions may be in the range of from about pH 5 to about pH 8, when administered. The compositions of the invention are sterile if they are to be used for therapeutic purposes. Sterility can be achieved by any of several means known in the art, including by filtration through sterile filtration membranes (e.g., 0.2 micron membranes). Sterility may be maintained with or without anti-bacterial agents.

### Exemplary Uses of IL13R/IL4R Contiguous Polypeptides, IL13Rd/IL4R Contiguous Polypeptides, IL13R/IL4R Heterodimeric Proteins, and IL13Rd/IL4R Heterodimeric Proteins and Pharmaceutical Compositions

The IL13R/IL4R contiguous polypeptides, IL13Rd/IL4R contiguous polypeptides, IL13R/IL4R heterodimeric proteins, and IL13Rd/IL4R heterodimeric proteins of the invention or pharmaceutical compositions comprising the contiguous polypeptides or heterodimeric proteins thereof may be useful for treating an IL13- and/or IL4-induced condition. As used herein, an “IL13- or IL4-induced condition” means a disease associated with, caused by, or characterized by, elevated levels or altered distribution of IL13 or IL4. Such IL13- and/or IL4-induced conditions include, but are not limited to, a pruritic or an allergic disease. In some embodiments, the IL13- and/or IL4-induced condition is atopic dermatitis, pruritus, asthma, psoriasis, scleroderma, or eczema. An IL13- or IL4-induced condition may be exhibited in a companion animal, including, but not limited to, canine, feline, or equine.

As used herein, “treatment” is an approach for obtaining beneficial or desired clinical results. “Treatment” as used herein, covers any administration or application of a therapeutic for disease in a mammal, including a companion animal. For purposes of this disclosure, beneficial or desired clinical results include, but are not limited to, any one or more of: alleviation of one or more symptoms, diminishment of extent of disease, preventing or delaying spread of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

In some embodiments, an IL13R/IL4R contiguous polypeptide, an IL13Rd/IL4R contiguous polypeptide, an IL13R/IL4R heterodimeric protein, or an IL13R/IL4R heterodimeric protein, or pharmaceutical compositions comprising it can be utilized in accordance with the methods herein to treat IL13- or IL4-induced conditions. In some embodiments, an IL13R/IL4R contiguous polypeptide, an IL13Rd/IL4R contiguous polypeptide, an IL13R/IL4R heterodimeric protein, or an IL13Rd/IL4R heterodimeric protein or a pharmaceutical composition is administered to a companion animal, such as a canine, a feline, or equine, to treat an IL13- and/or an IL4-induced condition.

A “therapeutically effective amount” of a substance/molecule, agonist or antagonist may vary according to factors such as the type of disease to be treated, the disease state, the severity and course of the disease, the type of therapeutic purpose, any previous therapy, the clinical history, the response to prior treatment, the discretion of the attending veterinarian, age, sex, and weight of the animal, and the ability of the substance/molecule, agonist or antagonist to elicit a desired response in the animal. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at dosages and for periods of time necessary, to achieve the desired therapeutic or prophylactic result.

In some embodiments, an IL13R/IL4R contiguous polypeptide, an IL13Rd/IL4R contiguous polypeptide, an IL13R/IL4R heterodimeric protein, or an IL13Rd/IL4R heterodimeric protein, or a pharmaceutical composition comprising a contiguous polypeptide or a heterodimeric protein thereof is administered parenterally, by subcutaneous administration, intravenous infusion, or intramuscular injection. In some embodiments, an IL13R/IL4R contiguous polypeptide, an IL13Rd/IL4R contiguous polypeptide, an IL13R/IL4R heterodimeric protein, an IL13Rd/IL4R heterodimeric protein, or pharmaceutical composition comprising a contiguous polypeptide or a heterodimeric protein thereof is administered as a bolus injection or by continuous infusion over a period of time. In some embodiments, an IL13R/IL4R contiguous polypeptide, an IL13Rd/IL4R contiguous polypeptide, an IL13R/IL4R heterodimeric protein, or an IL13Rd/IL4R heterodimeric protein, or pharmaceutical composition comprising a contiguous polypeptide or a heterodimeric protein thereof is administered by an intramuscular, an intraperitoneal, an intracerebrospinal, a subcutaneous, an intra-arterial, an intrasynovial, an intrathecal, or an inhalation route.

An IL13R/IL4R contiguous polypeptide, IL13Rd/IL4R contiguous polypeptide, an IL13R/IL4R heterodimeric protein, or an IL13Rd/IL4R heterodimeric protein described herein may be administered in an amount in the range of 0.1 mg/kg body weight to 100 mg/kg body weight per dose. In some embodiments, an IL13R/IL4R contiguous polypeptide, an IL13Rd/IL4R contiguous polypeptide, an IL13R/IL4R heterodimeric protein, or an IL13Rd/IL4R heterodimeric protein may be administered in an amount in the range of 0.1 mg/kg body weight to 50 mg/kg body weight per dose. In some embodiments, an IL13R/IL4R contiguous polypeptide, an IL13Rd/IL4R contiguous polypeptide, an IL13R/IL4R heterodimeric protein, or an IL13Rd/IL4R heterodimeric protein may be administered in an amount in the range of 1 mg/kg body weight to 10 mg/kg body weight per dose. In some embodiments, an IL13R/IL4R contiguous polypeptide, an IL13Rd/IL4R contiguous polypeptide, an IL13R/IL4R heterodimeric protein, or an IL13Rd/IL4R heterodimeric protein may be administered in an amount in the range of 0.5 mg/kg body weight to 100 mg/kg body, in the range of 1 mg/kg body weight to 100 mg/kg body weight, in the range of 5 mg/kg body weight to 100 mg/kg body weight, in the range of 10 mg/kg body weight to 100 mg/kg body weight, in the range of 20 mg/kg body weight to 100 mg/kg body weight, in the range of 50 mg/kg body weight to 100 mg/kg body weight, in the range of 1 mg/kg body weight to 10 mg/kg body weight, in the range of 5 mg/kg body weight to 10 mg/kg body weight, in the range of 0.5 mg/kg body weight to 10 mg/kg body weight, or in the range of 5 mg/kg body weight to 50 mg/kg body weight.

An IL13R/IL4R contiguous polypeptide, an IL13Rd/IL4R contiguous polypeptide, an IL13R/IL4R heterodimeric protein, or an IL13Rd/IL4R heterodimeric protein, or a pharmaceutical composition comprising a contiguous polypeptide or a heterodimeric protein thereof can be administered to a companion animal at one time or over a series of treatments. For example, an IL13R/IL4R contiguous polypeptide, an IL13Rd/IL4R contiguous polypeptide, an IL13R/IL4R heterodimeric protein, an IL13Rd/IL4R heterodimeric protein, or a pharmaceutical composition comprising a contiguous polypeptide or a heterodimeric protein thereof may be administered at least once, more than once, at least twice, at least three times, at least four times, or at least five times.

In some embodiments, the dose is administered once per week for at least two or three consecutive weeks, and in some embodiments, this cycle of treatment is repeated two or more times, optionally interspersed with one or more weeks of no treatment. In other embodiments, the therapeutically effective dose is administered once per day for two to five consecutive days, and in some embodiments, this cycle of treatment is repeated two or more times, optionally interspersed with one or more days or weeks of no treatment.

In some embodiments, a long-acting IL13R/IL4R contiguous polypeptide, IL13Rd/IL4R contiguous polypeptide, IL13R/IL4R heterodimeric protein, or IL13Rd/IL4R heterodimeric protein, is administered at a reduced dose and/or with an increased interval between dosing relative to a reference contiguous polypeptide or heterodimeric protein.

Administration “in combination with” one or more further therapeutic agents includes simultaneous (concurrent) and consecutive or sequential administration in any order. The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about a specified number of minutes. The term “sequentially” is used herein to refer to administration of two or more therapeutic agents where the administration of one or more agent(s) continues after discontinuing the administration of one or more other agent(s), or wherein administration of one or more agent(s) begins before the administration of one or more other agent(s). For example, administration of the two or more therapeutic agents are administered with a time separation of more than about a specified number of minutes. As used herein, “in conjunction with” refers to administration of one treatment modality in addition to another treatment modality. As such, “in conjunction with” refers to administration of one treatment modality before, during or after administration of the other treatment modality to the animal.

In some embodiments, the method comprises administering in combination with an IL13R/IL4R contiguous polypeptide, an IL13Rd/IL4R contiguous polypeptide, an IL13R/IL4R heterodimeric protein, or an IL13Rd/IL4R heterodimeric protein, or a pharmaceutical composition comprising a contiguous polypeptide or a heterodimeric protein thereof, a Jak inhibitor, a PI3K inhibitor, an AKT inhibitor, or a MAPK inhibitor. In some embodiments, the method comprises administering in combination with an IL13R/IL4R contiguous polypeptide, an IL13Rd/IL4R contiguous polypeptide, an IL13R/IL4R heterodimeric protein, or an IL13Rd/IL4R heterodimeric protein, or a pharmaceutical composition comprising a contiguous polypeptide or a contiguous polypeptide thereof, anti-IL31 antibody, an anti-IL4R antibody, an anti-IL17 antibody, an anti-TNFα antibody, an anti-CD20 antibody, an anti-CD19 antibody, an anti-CD25 antibody, an anti-IL31 antibody, an anti-IL23 antibody, an anti-IgE antibody, an anti-CD11a antibody, anti-IL6R antibody, anti-α4-Intergrin antibody, an anti-IL12 antibody, an anti-IL10P antibody, or an anti-BlyS antibody.

Provided herein are methods of exposing to a cell an IL13R/IL4R contiguous polypeptide, an IL13Rd/IL4R contiguous polypeptide, an IL13R/IL4R heterodimeric protein, or an IL13Rd/IL4R heterodimeric protein, or a pharmaceutical composition comprising a contiguous polypeptide or a heterodimeric protein thereof under conditions permissive for binding to IL13 and/or IL4. In some embodiments, the cell is exposed to the IL13R/IL4R contiguous polypeptide, IL13Rd/IL4R contiguous polypeptide, IL13R/IL4R heterodimeric protein, IL13Rd/IL4R heterodimeric protein, or pharmaceutical composition ex vivo. In some embodiments, the cell is exposed to the IL13R/IL4R contiguous polypeptide, IL13Rd/IL4R contiguous polypeptide, IL13R/IL4R heterodimeric protein, IL13Rd/IL4R heterodimeric protein, or pharmaceutical composition in vivo. In some embodiments, a cell is exposed to the IL13R/IL4R contiguous polypeptide, IL13Rd/IL4R contiguous polypeptide, IL13R/IL4R heterodimeric protein, IL13Rd/IL4R heterodimeric protein, or the pharmaceutical composition under conditions permissive for binding of the contiguous polypeptide or heterodimeric protein to extracellular IL13 and/or IL4. In some embodiments, a cell may be exposed in vivo to the IL13R/IL4R contiguous polypeptide, IL13Rd/IL4R contiguous polypeptide, IL13R/IL4R heterodimeric protein, IL13Rd/IL4R heterodimeric protein, or the pharmaceutical composition by any one or more of the administration methods described herein, including but not limited to, intraperitoneal, intramuscular, intravenous injection into the subject. In some embodiments, a cell may be exposed ex vivo to the IL13R/IL4R contiguous polypeptide, IL13Rd/IL4R contiguous polypeptide, IL13R/IL4R heterodimeric protein, IL13Rd/IL4R heterodimeric protein, or the pharmaceutical composition by exposing the cell to a culture medium comprising the heterodimeric protein or the pharmaceutical composition. In some embodiments, the permeability of the cell membrane may be affected using any number of methods understood by those of skill in the art (such as electroporating the cells or exposing the cells to a solution containing calcium chloride) before exposing the cell to a culture medium comprising the fusion molecule or the pharmaceutical composition.

In some embodiments, the exposure results in a reduction of IL13 and/or IL4 signaling function by the cell. In some embodiments, an IL13R/IL4R contiguous polypeptide, an IL13Rd/IL4R contiguous polypeptide, an IL13R/IL4R heterodimeric protein, or an IL13Rd/IL4R heterodimeric protein may reduce IL13 and/or IL4 signaling in a cell by at least 10%, at least 15%, at least 20%, at least 25%, at least 30%, at least 35%, at least 40%, at least 45%, at least 50%, at least 60%, at least 70%, at least 80%, at least 90%, or 100% compared to IL13 and/or IL4 signaling function in the absence of the contiguous polypeptide or heterodimeric protein. In some embodiments, the reduction in IL13 and/or IL4 signaling and/or the reduction in TF-1 proliferation is between 10% and 15%, between 10% and 20%, between 10% and 25%, between 10% and 30%, between 10% and 35%, between 10% and 40%, between 10% and 45%, between 10% and 50%, between 10% and 60%, between 10% and 70%, between 10% and 80%, between 10% and 90%, between 10% and 100%, between 15% and 20%, between 15% and 25%, between 15% and 30%, between 15% and 35%, between 15% and 40%, between 15% and 45%, between 15% and 50%, between 15% and 60%, between 15% and 70%, between 15% and 80%, between 15% and 90%, between 15% and 100%, between 20% and 25%, between 20% and 30%, between 20% and 35%, between 20% and 40%, between 20% and 45%, between 20% and 50%, between 20% and 60%, between 20% and 70%, between 20% and 80%, between 20% and 90%, between 20% and 100%, between 25% and 30%, between 25% and 35%, between 25% and 40%, between 25% and 45%, between 25% and 50%, between 25% and 60%, between 25% and 70%, between 25% and 80%, between 25% and 90%, between 25% and 100%, between 30% and 35%, between 30% and 40%, between 30% and 45%, between 30% and 50%, between 30% and 60%, between 30% and 70%, between 30% and 80%, between 30% and 90%, between 30% and 100%, between 35% and 40%, between 35% and 45%, between 35% and 50%, between 35% and 60%, between 35% and 70%, between 35% and 80%, between 35% and 90%, between 35% and 100%, between 40% and 45%, between 40% and 50%, between 40% and 60%, between 40% and 70%, between 40% and 80%, between 40% and 90%, between 40% and 100%, between 45% and 50%, between 45% and 60%, between 45% and 70%, between 45% and 80%, between 45% and 90%, between 45% and 100%, between 50% and 60%, between 50% and 70%, between 50% and 80%, between 50% and 90%, between 50% and 100%, between 60% and 70%, between 60% and 80%, between 60% and 90%, between 60% and 100%, between 70% and 80%, between 70% and 90%, between 70% and 100%, between 80% and 90%, between 80% and 100%, or between 90% and 100%.

Provided herein are methods of using the IL13R/IL4R contiguous polypeptide, IL13Rd/IL4R contiguous polypeptide, IL13R/IL4R heterodimeric protein, or IL13Rd/IL4R heterodimeric protein for detection, diagnosis and monitoring of an IL13- or IL4-induced condition. Provided herein are methods of determining whether a companion animal will respond to IL13R/IL4R contiguous polypeptide, IL13Rd/IL4R contiguous polypeptide, IL13R/IL4R heterodimeric protein, or IL13Rd/IL4R heterodimeric protein therapy. In some embodiments, the method comprises detecting whether the animal has cells that express IL13 or IL4 using an IL13R/IL4R contiguous polypeptide, an IL13Rd/IL4R contiguous polypeptide, an IL13R/IL4R heterodimeric protein, or an IL13Rd/IL4R heterodimeric protein. In some embodiments, the method of detection comprises contacting the sample with an antibody, polypeptide, or polynucleotide and determining whether the level of binding differs from that of a reference or comparison sample (such as a control). In some embodiments, the method may be useful to determine whether the IL13R/IL4R contiguous polypeptide, IL13Rd/IL4R contiguous polypeptide, IL13R/IL4R heterodimeric protein, or IL13Rd/IL4R heterodimeric protein described herein are an appropriate treatment for the subject animal.

In some embodiments, the sample is a biological sample. The term “biological sample” means a quantity of a substance from a living thing or formerly living thing. In some embodiments, the biological sample is a cell or cell/tissue lysate. In some embodiments, the biological sample includes, but is not limited to, blood, (for example, whole blood), plasma, serum, urine, synovial fluid, and epithelial cells.

In some embodiments, the cells or cell/tissue lysate are contacted with an IL13R/IL4R contiguous polypeptide, an IL13Rd/IL4R contiguous polypeptide, an IL13R/IL4R heterodimeric protein, or an IL13Rd/IL4R heterodimeric protein and the binding between the contiguous polypeptide or the heterodimeric protein and the cell is determined. When the test cells show binding activity as compared to a reference cell of the same tissue type, it may indicate that the subject would benefit from treatment with an IL13R/IL4R contiguous polypeptide, an IL13Rd/IL4R contiguous polypeptide, an IL13R/IL4R heterodimeric protein, or an IL13Rd/IL4R heterodimeric protein. In some embodiments, the test cells are from tissue of a companion animal.

Various methods known in the art for detecting specific antibody-antigen binding can be used. Exemplary immunoassays which can be conducted include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected to meet the needs of various uses of the method which are often dictated by the availability of assay equipment and compatible immunoassay procedures. Appropriate labels include, without limitation, radionuclides (for example ¹²⁵I, ¹³¹I, ³⁵S, ³H, or ³²P), enzymes (for example, alkaline phosphatase, horseradish peroxidase, luciferase, or p-galactosidase), fluorescent moieties or proteins (for example, fluorescein, rhodamine, phycoerythrin, GFP, or BFP), or luminescent moieties (for example, Qdot™ nanoparticles supplied by the Quantum Dot Corporation, Palo Alto, Calif.). General techniques to be used in performing the various immunoassays noted above are known to those of ordinary skill in the art.

For purposes of diagnosis, the IL13R/IL4R contiguous polypeptide, IL13Rd/IL4R contiguous polypeptide, IL13R/IL4R heterodimeric protein, or IL13Rd/IL4R heterodimeric protein can be labeled with a detectable moiety including but not limited to radioisotopes, fluorescent labels, and various enzyme-substrate labels know in the art. Methods of conjugating labels to polypeptides are known in the art. In some embodiments, the contiguous polypeptide or the heterodimeric protein need not be labeled, and the presence thereof can be detected, for example, using an antibody that binds to the contiguous polypeptide or heterodimeric protein. In some embodiments, the IL13R/IL4R contiguous polypeptide, IL13Rd/IL4R contiguous polypeptide, IL13R/IL4R heterodimeric protein, or IL13Rd/IL4R heterodimeric protein can be employed in any known assay method, such as competitive binding assays, direct and indirect sandwich assays, and immunoprecipitation assays. Zola, Monoclonal Antibodies: A Manual of Techniques, pp. 147-158 (CRC Press, Inc. 1987). The anti-IL13 and IL4 antibodies and polypeptides can also be used for in vivo diagnostic assays, such as in vivo imaging. Generally, the antibody or the polypeptide is labeled with a radionuclide (such as ¹¹¹In, ⁹⁹Tc, ¹⁴C, ¹³¹I, ¹²⁵I, ³H, or any other radionuclide label, including those outlined herein) so that the cells or tissue of interest can be localized using immunoscintiography. The contiguous polypeptide or heterodimeric protein may also be used as staining reagent in pathology using techniques well known in the art.

In some embodiments, an IL13R/IL4R contiguous polypeptide, an IL13Rd/IL4R contiguous polypeptide, an IL13R/IL4R heterodimeric protein, or an IL13Rd/IL4R heterodimeric protein is used for a diagnostic and an IL13R/IL4R contiguous polypeptide, IL13Rd/IL4R contiguous polypeptide, an IL13R/IL4R heterodimeric protein, or an IL13Rd/IL4R heterodimeric protein is used as a therapeutic. In some embodiments, the diagnostic protein and the therapeutic protein are different. In some embodiments, the diagnostic protein and the therapeutic protein are the same.

The following examples illustrate particular aspects of the disclosure and are not intended in any way to limit the disclosure.

### EXAMPLES

### Example 1

### Expression and Purification of Canine IL4 and IL13

A nucleotide sequence encoding canine IL13 protein (SEQ ID NO: 4) was synthesized with poly-His tag on the C-terminal end and cloned into a mammalian expression vector and transfected to 293 cells or CHOS. The same method was used to clone and express a nucleotide sequence encoding canine IL4 protein (SEQ ID NO: 1) with a poly-His tag on the C-terminal end.

The supernatant containing canine IL13 protein was collected and filtered. Canine IL13 was affinity purified using Ni-NTA column (CaptivA® Protein A Affinity Resin, Repligen). The same method was used to purify canine IL4.

### Example 2

### Extracellular Domains of IL13R and IL4R

Extracellular domains of canine, feline, and equine IL4R that are responsible for binding canine, feline and equine IL4 and/or IL13 were identified and boundaries were defined. Exemplary full-length extracellular domains of canine IL4R, feline IL4R, and equine IL4 were identified as SEQ ID NO: 23 and SEQ ID NO: 163 (canine), SEQ ID NO: 25 (feline), and SEQ ID NO: 27 (equine). Exemplary extracellular domain fragments of canine IL4R, feline IL4R, and equine IL4R postulated to retain biological activity were identified as SEQ ID NO: 33, SEQ ID NO: 35, and SEQ ID NO: 37, respectively.

Extracellular domains of canine, feline, and equine IL13R that are responsible for binding canine, feline, and equine IL4 and/or IL13 were identified and boundaries were defined. Exemplary full-length extracellular domains of canine IL13R, feline IL13R, and equine IL13R were identified as SEQ ID NO: 22, SEQ ID NO: 24, and SEQ ID NO: 26, respectively. Exemplary extracellular domain fragments of canine IL13R, feline IL13R, and equine IL13R postulated to retain biological activity were identified as SEQ ID NO: 32, SEQ ID NO: 34, and SEQ ID NO: 36, respectively.

An unpaired cysteine (Cys) in canine IL13R (at position 18 of SEQ ID NO: 22), feline IL13R (at position 18 of SEQ ID NO: 24), and equine IL13R (at position 18 of SEQ ID NO: 26) was identified informatically and determined as embedded (unexposed) based on 3-D modeling. It is unlikely that the unpaired cysteine will form disulfide bonds and the likelihood of aggregation is low. Thus, site-directed mutagenesis of this Cys residue was not introduced.

### Example 3

Expression and Purification of Canine IL13R/IL4R Contiguous Polypeptides from CHO Cells

Nucleotide sequences encoding canine IL13R ECD/IL4R ECD contiguous polypeptides linked to an IgGB Fc polypeptide were designed with a signal sequence. For contiguous polypeptide “IL13RECD-IL4RECD-IgGB Fc” (SEQ ID NO: 20), an extracellular domain of IL13R (SEQ ID NO: 22) precedes an extracellular domain of IL4R (SEQ ID NO: 23). For contiguous polypeptide “IL4RECD-IL13RECD-IgGB Fc” (SEQ ID NO: 21), an extracellular domain of IL4R precedes an extracellular domain of IL13R.

The nucleotide sequences were synthesized chemically and inserted into an expression vector suitable for transfection into a CHO host cell. After transfection into CHO cells, the fusion proteins were secreted from the cell. For example, fusion protein was purified by single step Protein A column chromatography.

Each of IL13RECD-IL4RECD-IgGB Fc and IL4RECD-IL13RECD-IgGB Fc may be expressed and purified in a single step with a protein A column or other chromatographic methods, such as ion exchange column chromatography, hydrophobic interaction column chromatography, mixed mode column chromatography such as CHT, or multimodal mode column chromatography such as CaptoMMC. Low pH or other viral inactivation and viral removal steps can be applied. The purified protein may be admixed with excipients, and sterilized by filtration to prepare a pharmaceutical composition of the invention. The pharmaceutical composition may be administered to a dog with an atopic dermatitis or asthma in an amount sufficient to bind and/or inhibit either IL13 and/or IL4.

The vectors were then used to perform pilot-scale transfection in CHO-S cells using the FreestyleMax™ transfection reagent (Life Technologies). The supernatant was harvested by clarifying the conditioned media. Protein was purified with a single pass Protein A chromatography step and used for further investigation.

### Example 4

### Demonstration of IL13 and IL4 Binding Activity

This example demonstrates that both IL13RECD-IL4RECD-IgGB Fc (SEQ ID NO:20) and IL4RECD-IL13RECD-IgGB Fc (SEQ ID NO:21) bind canine IL4 and IL13 with kinetics requisite for therapeutic activity.

The binding analysis was performed using a biosensor Octet as follows. Briefly, canine IL4 (produced using 293 cells) was biotinylated. The free unreacted biotin was removed from biotinylated IL4 by extensive dialysis. Biotinylated canine IL4 was captured on streptavidin sensor tips. The IL4 association with various concentrations (12, 16, and 44 nM) of IL13RECD-IL4RECD-IgGB Fc (SEQ ID NO:20) was monitored for ninety seconds. Dissociation was monitored for 600 seconds. A buffer only blank curve was subtracted to correct for any drift. The data were fit to a 1:1 binding model using ForteBio™ data analysis software to determine the kₒₙ, k_{off}, and the Kd. The buffer for dilutions and all binding steps was: 20 mM phosphate, 150 mM NaCl, pH 7.2. The Kd for IL13RECD-IL4RECD-IgGB Fc and ligand IL4 was 8×10⁻¹¹.

The canine IL4 association with various concentrations (40.7, and 140 nM) of IL4RECD-IL13RECD-IgGB Fc (SEQ ID NO:21) was monitored for ninety seconds. Dissociation was monitored for 600 seconds. A buffer only blank curve was subtracted to correct for any drift. The data were fit to a 1:1 binding model using ForteBio™ data analysis software to determine the kₒₙ, k_{off}, and the Kd. The buffer for dilutions and all binding steps was: 20 mM phosphate, 150 mM NaCl, pH 7.2. The Kd for IL4RECD-IL13RECD-IgGB Fc and ligand IL4 was 1.1×10⁻¹¹.

Canine IL4 and canine IL13 with C-terminal polyHis tag was expressed and purified from 293 cells. EZ-Link NHS-LC-biotin was obtained from Thermo Scientific (Cat. #21336), and Streptavidin biosensors was obtained from ForteBio (Cat. #18-509).

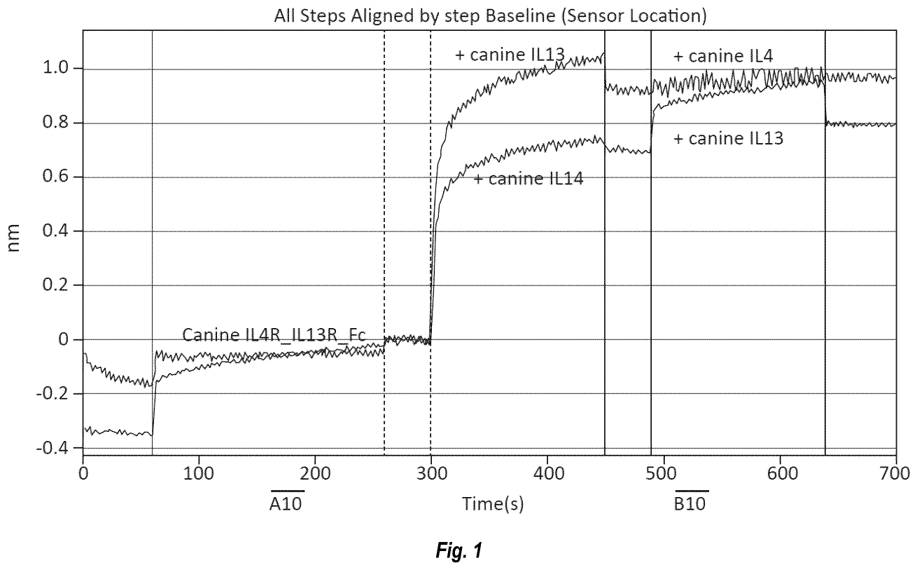

IL4 and IL13 sequential binding experiments with IL13RECD-IL4RECD-IgGB Fc (SEQ ID NO:20) were performed. Biotinylated canine IL13RECD-IL4RECD-IgGB Fc was captured on streptavidin sensor tips. Canine IL13RECD-IL4RECD-IgGB Fc was exposed to either (1) canine IL4 followed by IL13 or (2) canine IL13 followed by IL4 using concentrations of 30 μg/mL of IL4 and IL13 in PBS (FIG. 1). The experiments demonstrated that once IL13RECD-IL4RECD-IgGB Fc bound to IL13, it may not bind to IL4, and that once bound to IL4, its ability to bind IL13 is reduced.

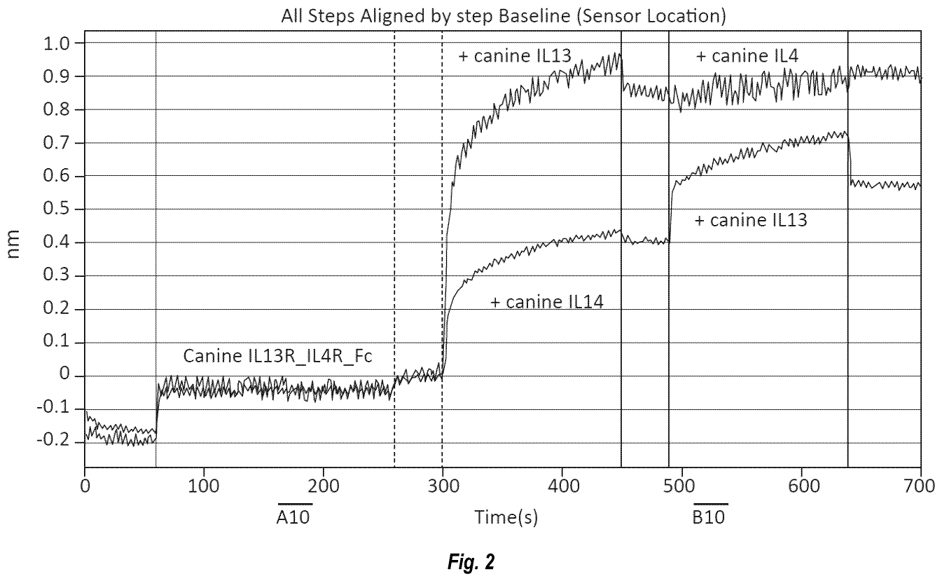

IL4 and IL13 sequential binding experiments with IL4RECD-IL13RECD-IgGB Fc (SEQ ID NO:21) were performed. Biotinylated canine IL4RECD-IL13RECD-IgGB Fc was captured on streptavidin sensor tips. Canine IL4RECD-IL13RECD-IgGB Fc was exposed to either (1) canine IL4 followed by IL13 or (2) canine IL13 followed by IL4 using concentrations of 30 μg/mL of IL4 and IL13 in PBS (FIG. 2). These experiments demonstrated that once IL4RECD-IL13RECD-IgGB Fc bound to IL13, it may not bind to IL4, and that once bound to IL4, its ability to bind IL13 is reduced.

The tight binding of IL13RECD-IL4RECD-IgGB Fc and IL4RECD-IL13RECD-IgGB Fc to IL4 or IL13 is thought to be due to simultaneous binding contributions made by both IL4RECD and IL13RECD.

### Example 5

### Cellular Functional Activity of Canine IL4RECD-IL13RECD-Fc (SINK)

TF1 cells (ATCC cat #CRL-2003), a human Erythroleukemic cell line which expresses endogenous interleukin 4 receptors on cell surface, was used in a proliferation assay. Cells grown in RPMI1640 (Gibco, Cat #11875) supplemented with 10% Fetal Bovine Serum, heat inactivated (Sigma, Cat #2868) and 2 nM/ml Human GM-CSF (R&D System, Cat #215-GM-010) at exponential growth phase were used for the assay. Cells were washed with PBS twice and resuspended in above medium without GM-CSF. 20,000 cells per well were plated in a 96-well plate (Corning, Cat #3610). Canine IL4RECD-IL13RECD-IgGB Fc (SINK) was added at a series of dilutions followed by addition of canine IL4 (Sino Biological Inc, Cat #70021-DNAE-5) at 50 ng/ml. The cells were incubated in 37° C., 5% CO2 for 48 hours in a total volume of 100 μl. At the end of the incubation, the cells were cooled in room temperature and assayed for proliferation/variability by measuring cellular ATP content using CellTiter-Glo® Luminescent Cell Viability Assay (Promega, Cat #G7570).

In this assay, 100 μl premixed reagent A and B were added to each well. After shaking on an orbital shaker for 2 mins, the cells were lysed. Mono-oxygenation of luciferin was catalyzed by luciferase in the presence of Mg2+ and ATP that presented in cells, resulting in the generation of a luminescent signal proportional to the amount of ATP in the cells. The amount of ATP is directly proportional to the number of cells present in culture. The plate was incubated at room temperature for 10 minutes to stabilize the luminescent signal and luminescence was detected using a Synergy HT microplate reader (Biotek, Winooski, VT).

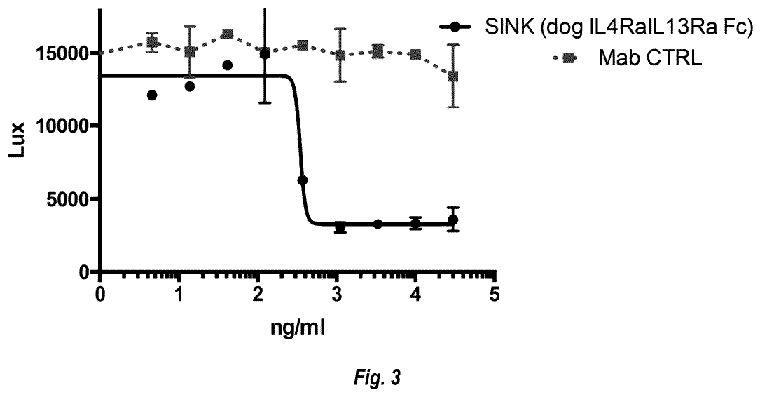

The data were analyzed using 4 parameter logistic fit and IC50 is 2.0 nM. See FIG. 3.

### Example 6

### Canine, Feline, and Equine IgG Fc Polypeptides for IL13R and IL4R Heterodimeric Proteins

Pairs of variant canine IgG Fc polypeptides, variant feline IgG Fc polypeptides, and variant equine IgG Fc polypeptides were designed such that a knob-in-hole heterodimerization approach may be used to prepare heterodimeric proteins comprising at least one IL13R ECD and at least one IL4R ECD. First, pairing of two Fc polypeptides was designed by introducing CH3 interfacing mutations so that a first Fc polypeptide comprises a bulky amino acid (knob) and a second Fc polypeptide comprises smaller amino acids in the same general location (hole).

An amino acid substitution of threonine to tryptophan at a position corresponding to position 138 of canine IgG-A (SEQ ID NO: 38), at a position corresponding to position 137 of canine IgG-B Fc (SEQ ID NO: 39), at a position corresponding to position 137 of canine IgG-C Fc (SEQ ID NO: 40), or at a position corresponding to position 138 of canine IgG-D Fc (SEQ ID NO: 41) (T138W or T137W) can be introduced as a knob. Examples of amino acid sequences of a first variant canine IgG-A, IgG-B, IgG-C, and IgG-D Fc polypeptide comprising a knob mutation are SEQ ID NO: 54, SEQ ID NO: 55, SEQ ID NO: 56, and SEQ ID NO: 57, respectively.

An amino acid substitution of threonine to serine at a position corresponding to position 138 and/or of leucine to alanine at a position corresponding to position 140 and/or of tyrosine to threonine at a position corresponding to position 180 of canine IgG-A (SEQ ID NO: 38) or of IgG-D (SEQ ID NO: 41) (T138S, L140A, and/or Y180T); or of threonine to serine at a position corresponding to position 137 and/or of leucine to alanine at a position corresponding to position 139 and/or of tyrosine to threonine at a position corresponding to position 179 of canine IgG-B Fc (SEQ ID NO: 39) or of IgG-C(SEQ ID NO: 40) (T137S, L139A, and/or Y179T) can be introduced as a hole. Examples of amino acid sequences of a second variant canine IgG-A, IgG-B, IgG-C, and IgG-D Fc polypeptides comprising a hole mutation are SEQ ID NO: 58, SEQ ID NO: 59, SEQ ID NO: 60, SEQ ID NO: 61, SEQ ID NO: 62, SEQ ID NO: 63, SEQ ID NO: 64, and SEQ ID NO: 65.

An amino acid substitution of threonine to tryptophan at a position corresponding to position 154 of feline IgG1a Fc (SEQ ID NO: 42 or SEQ ID NO: 43), feline IgG1b Fc (SEQ ID NO: 44 or SEQ ID NO: 45), or of feline IgG2 (SEQ ID NO: 46) (T154W) can be introduced as a knob. Examples of amino acid sequences of a first variant feline IgG1a, and IgG1b, and IgG2 Fc polypeptide comprising a knob mutation are SEQ ID NO: 66, SEQ ID NO: 67, SEQ ID NO: 68, SEQ ID NO: 69, and SEQ ID NO: 70.

An amino acid substitution of threonine to serine at a position corresponding to position 154 and/or of leucine to alanine at a position corresponding to position 156 and/or of tyrosine to threonine at a position corresponding to position 197 of feline IgG1a (SEQ ID NO: 42 or SEQ ID NO: 43), feline IgG-b Fc (SEQ ID NO: 44 or SEQ ID NO: 45), or feline IgG2 Fc (SEQ ID NO: 46) (T154S, L156A, and/or Y(197)T) can be introduced as a hole. Examples of amino acid sequences of a second variant feline IgG1a, IgG1b, IgG2 Fc polypeptide comprising a hole mutation are SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, and SEQ ID NO: 80.

An amino acid substitution of threonine to tryptophan at a position corresponding to position 130 of equine IgG1 Fc (SEQ ID NO: 47), of equine IgG2 Fc (SEQ ID NO: 48), of equine IgG3 Fc (SEQ ID NO: 49), of equine IgG4 Fc (SEQ ID NO: 50), of equine IgG5 Fc (SEQ ID NO: 51), of equine IgG6 Fc (SEQ ID NO: 52), or of equine IgG7 Fc (SEQ ID NO: 53)(T130W) can be introduced as a knob. Examples of amino acid sequences of a first variant equine IgG1, IgG2, IgG3, IgG4, IgG5, IgG6, and IgG7 Fc polypeptides comprising a knob mutation are SEQ ID NO: 81, SEQ ID NO: 82, SEQ ID NO: 83, SEQ ID NO: 84, SEQ ID NO: 85, SEQ ID NO: 86, and SEQ ID NO: 87, respectively.

An amino acid substitution of threonine to serine at a position corresponding to position 130 and/or of leucine to alanine at a position corresponding to position 132 and/or of tyrosine to threonine at a position corresponding to position 173 of equine IgG1 Fc (SEQ ID NO: 47), of equine IgG2 Fc (SEQ ID NO: 48), of equine IgG3 Fc (SEQ ID NO: 49), of equine IgG4 Fc (SEQ ID NO: 50), of equine IgG5 Fc (SEQ ID NO: 51), of equine IgG6 Fc (SEQ ID NO: 52), or of equine IgG7 Fc (SEQ ID NO: 53) (T130W, L(132)A, and/or Y(173)T) can be introduced as a hole. Examples of amino acid sequences of a second variant equine IgG1, IgG2, IgG3, IgG4, IgG5, IgG6, and IgG7 Fc polypeptides comprising a hole mutation are SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, and SEQ ID NO: 101.

### Example 7

### IL13R/IL4R ECD Heterodimeric Proteins

In addition to contiguous IL13R/IL4R ECD polypeptide formats, heterodimeric protein pairs may have the following formats:
Heterodimeric protein A:
   Polypeptide 1: IL13R(n)-L-Fc1 and
   Polypeptide 2: IL4R(n)-L-Fc2; or
Heterodimeric protein B:
   Polypeptide 1: IL4R(n)-L-Fc1 and
   Polypeptide 2: IL13R(n)-L-Fc2,
      wherein IL13R(n) is at least one IL13R extracellular domain (ECD) polypeptide from a companion animal species, IL4R(n) is at least one IL4R ECD polypeptide from a companion animal species, (n) is one, two, three, four, or more ECD polypeptides, L is an optional linker, Fc1 is a variant Fc polypeptide, such as a variant Fc polypeptide comprising knob mutation, Fc2 is a variant Fc polypeptide, such as a variant Fc polypeptide comprising a hole mutation. An optional linker could also be used between multiple ECD polypeptides. In addition, other binding partner(s) may be included before, after, and/or between any one or more ECD polypeptide(s). Other potential binding partners include: IL5, IL6, IL17, IL22, IL31, LFA-1, TNF-α, TSLP, and/or IgE.

Examples of pairs of contiguous polypeptide 1 and 2 that may form a heterodimeric protein include SEQ ID NOs 102 and 103, SEQ ID NOs: 104 and 105, SEQ ID NOs: 106 and 107, SEQ ID NOs: 108 and 109, SEQ ID NOs: 110 and 111, and SEQ ID NOs: 112 and 113. A host cell may be cotransfected with vectors expressing these contiguous polypeptide pairs to produce the heterodimeric proteins described.

### Example 8

Screening Variant Canine IgG-B Polypeptides with Enhanced Canine FcRn/B2M Binding

Canine FcRn with a poly-His tag (SEQ ID NO: 114) and canine B2M (SEQ ID NO: 115) heterodimer complex was transiently expressed in HEK cells and purified using Ni-NTA chromatography.

Fast Screening for Expression, Biophysical Properties and Affinity (FASEBA) of canine IgG-B Fc phage libraries was performed. Briefly, the open reading frame of canine IgG-B Fc polypeptide was subcloned into plasmid pFASEBA. Based on three-dimensional protein modeling of the canine IgG-B/canine FcRn/canine B2M complex, twelve amino acid positions of canine IgG-B were identified as being potentially involved in the binding between IgG-B and FcRn/B2M. The twelve positions of canine IgG-B identified were Thr(21), Leu(22), Leu(23), Ile(24), Ala(25), Thr (27), Gly (80), His (81), Gln (82), Leu (85), Met (201), and Asn (207) of SEQ ID NO: 39.

Twelve single site NNK mutation libraries of canine IgG-B Fc were prepared such that each library should have included variant IgG-B Fc polypeptides having each of the 20 possible amino acids substituted at each of the twelve sites. Each phage library was panned against canine FcRn/B2M complex at pH 6.0. After three rounds of panning, a total of 53 Fc phage clones were identified as potentially having enhanced FcRn/B2M binding and the mutations were identified by sequencing.

Single E. coli colonies expressing each of the 53 variant canine IgG-B Fc polypeptides with an SASA tag were cultured and induced to express the Fc polypeptides. Cell culture media containing the variant canine IgG-B Fc polypeptides was exposed to immobilized BSA either on a plate or a Biacore chip. The plates or chips with bound variant canine IgG-B Fc polypeptides were exposed to soluble canine FcRn/B2M complex to screen for slow off rate (koff) at pH 6. Each variant IgG-B Fc polypeptide exhibiting a slower koff with canine FcRn/B2M complex compared to wildtype IgG-B Fc polypeptide was identified. Four lead variant canine IgG-B polypeptides were identified: L(23)Y (SEQ ID NO: 117; “Y00”); L(23)F (SEQ ID NO: 116; “F00”); L(23)M; and L(23)S.

The koff of each of the lead variant canine IgG-B polypeptides was further investigated. Biotinylated canine FcRn/B2M complex was immobilized on a Biacore chip and exposed to each variant canine IgG-B polypeptide as an analyte using a Biacore T200 at pH 6.0. The koff(1/s) for wild-type canine IgG-B Fc polypeptide was 1.22×10⁻¹; the koff (1/s) for variant canine IgG-B Fc polypeptide L(23)Y (“Y00”) was 1.38×10⁻²; the koff(1/s) for variant IgG-B Fc polypeptide L(23)F (“F00”) was 6.31×10⁻² and 8.47×10⁻²; the koff (1/s) for variant canine IgG-B polypeptide L(23)M was 1.26×10⁻¹; and the koff (1/s) for variant canine IgG-B polypeptide L(23)S was 2.41×10⁻¹.

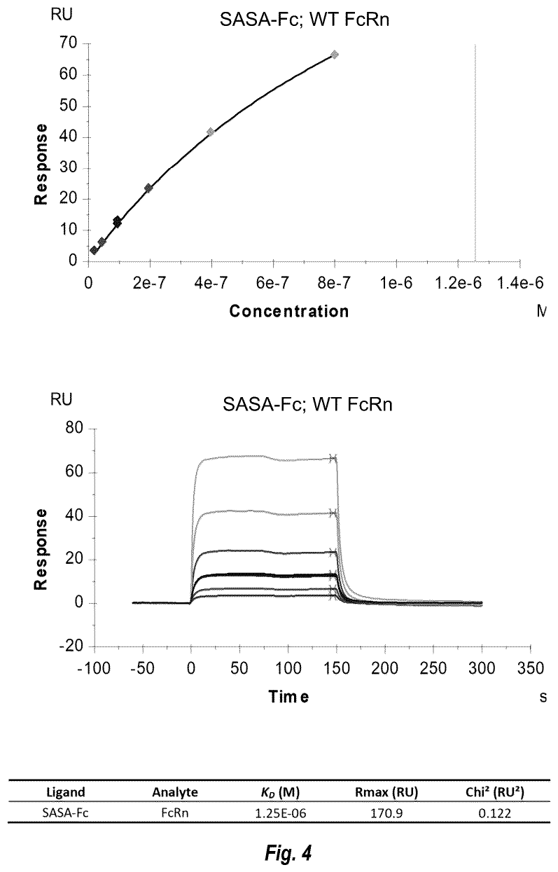

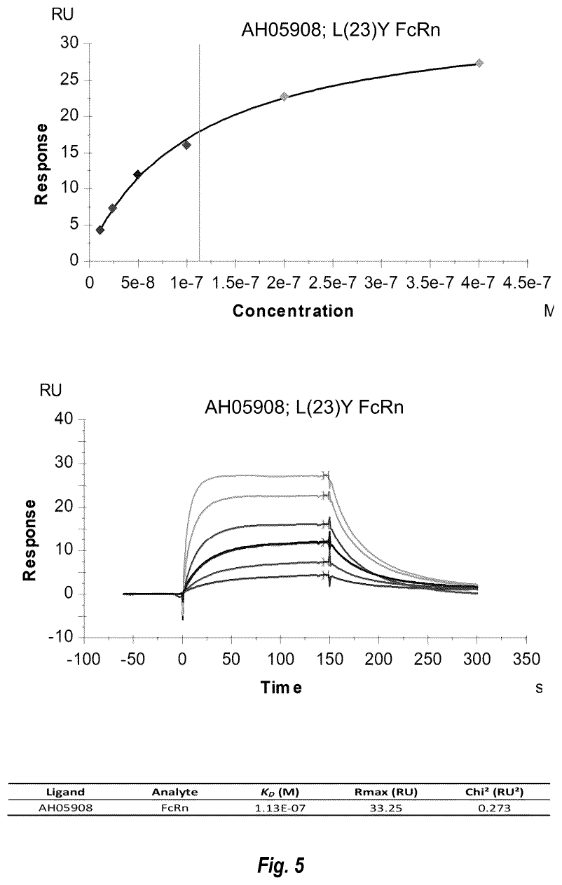

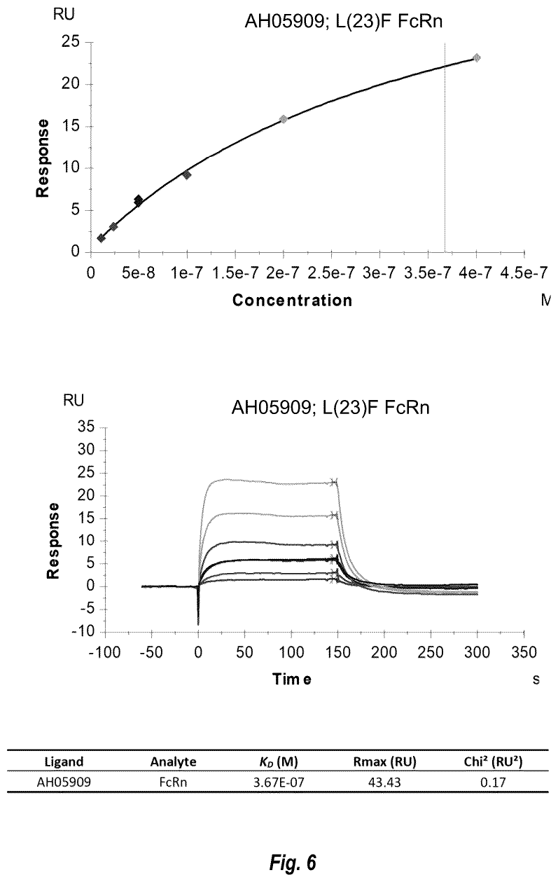

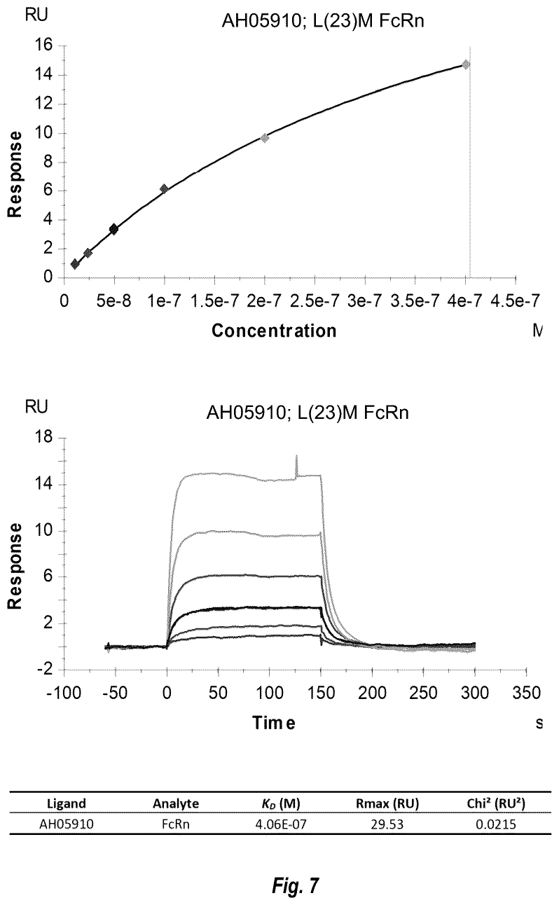

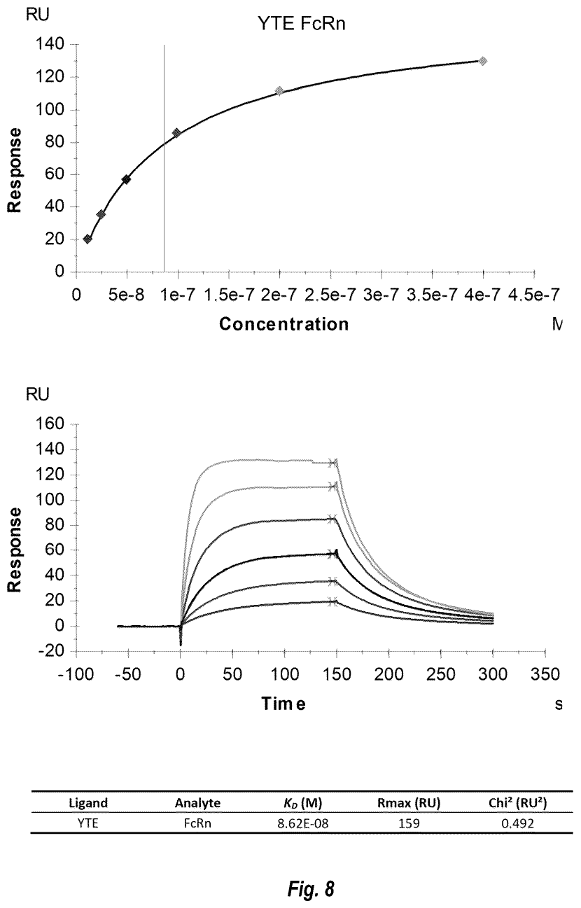

Binding analysis was performed using a Biacore T200. Briefly, the lead variant canine IgG-B Fc polypeptides with an SASA tag were each immobilized to a Series S Sensor Chip CM5. Association of each variant IgG-B Fc polypeptide with various concentrations of canine FcRn/B2M complex (12.5, 25, 50, 100, and 200 nM) was monitored at 25° C. until steady state was reached. A running buffer of 10 mM HEPES, 500 mM NaCl, 3 mM EDTA, 0.005% Tween-20, pH 6.0 was used. A buffer only blank curve was used as a control. The results are presented in FIGS. 10-14. The steady state Kd for wild-type canine IgG-B Fc polypeptide was 1.25×10⁻⁶ (FIG. 4); the steady state Kd for variant canine IgG-B Fc polypeptide L(23)Y (“Y00”) was 1.13×10⁻⁷ (FIG. 5); the steady state Kd for variant canine IgG-B Fc polypeptide L(23)F (“F00”) was 3.67×10⁻⁷ (FIG. 6); and the steady state Kd for variant canine IgG-B Fc polypeptide L(23)M was 4.06×10⁻⁷ (FIG. 7); and the steady state Kd for variant canine IgG-B Fc polypeptide YTE was 8.62×10⁻⁸ (FIG. 8).

### Example 9

### Phe Mutation in Canine IgG Enhances Canine FcRn Interaction

The affinity of variant canine Fc polypeptides for FcRn was evaluated in the context of a chimeric antibody. Antibody variable light chains fused to canine kappa light chain and variable heavy chains fused to variant canine IgG-A Fc polypeptides comprising SEQ ID NO: 118 (F00; Protein A+; C1q−; CD16−) or SEQ ID NO: 119 (Protein A+; C1q+; CD16+) and to variant canine IgG-D Fc polypeptides comprising SEQ ID NO: 120 (F00; Protein A+; C1q−; CD16−), or SEQ ID NO: 121 (Protein A+; C1q+; CD16+) were expressed.

The binding analysis was performed using a biosensor OctetRed as follows. Briefly, biotinylated TNFα was captured on streptavidin sensor tips. The association of antibody at 20 μg/mL was bound to TNFα. The complex was then used to bind to canine FcRn (50 μg/mL) at pH 6.0. Dissociation was performed at pH 7.2.

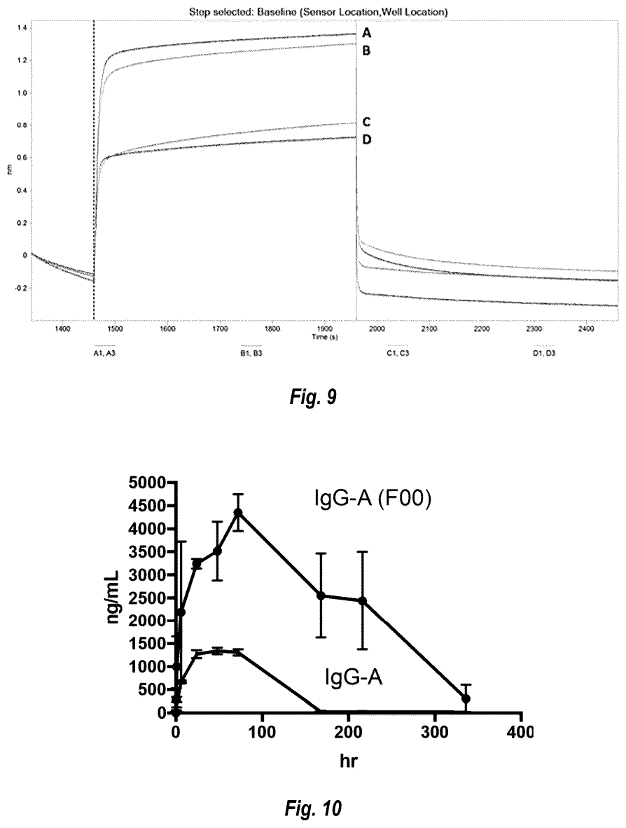

The Phe mutation enhanced canine FcRn binding at low pH (pH6.0, 20 mM NaCitrate, 140 mM NaCl), as illustrated by the binding profiles of chimeric variant canine IgG-A “F00” antibody (FIG. 9, A) and IgG-D “F00” antibody (FIG. 9, B) compared to chimeric variant canine IgG-A without the Phe mutation (FIG. 9, C) and IgG-D without the Phe mutation (FIG. 9, D). The chimeric variant canine IgG-A and IgG-D antibodies with the Phe mutation (FIG. 9, A and B) exhibited enhanced association with canine FcRn at low pH (pH 6.0) and fast dissociation at neutral pH (PBS pH7.2). A similar enhanced binding profile was also observed with chimeric variant canine IgG-B “F00” antibody.

### Example 10

### Pharmacokinetics of Phe Mutation in Canine IgG

Pharmacokinetics analysis was performed using Sprague Dawley rats. The rats were subcutaneously administered with 2 mg/kg of chimeric variant canine IgG-A “F00” antibody and chimeric variant canine IgG-A without the Phe mutation (two rats per group). Serum samples were collected from the rats at pre-injection and at 0.5, 1, 6, 24, 48, 72, 168, 216, and 336 hours post injection. The canine chimeric antibody concentrations in the serum samples were determined by ELISA, as follows.

Capture antibody (1 μg/mL in PBS) was coated on a 96-well Maxisorp plate with 100 μl in each well. The plate was incubated overnight at 4° C. and washed five times with PBST (PBS containing 0.05% Tween-20). Each well was blocked with 200 μl 5% BSA in PBST and the plate incubated for 1 hour at room temperature. The plate was washed five times with PBST. Dilutions of control antibody (1,000 ng/mL to 0.1 ng/mL) were added to the plate in duplicate and along with a blank well containing no control antibody were used to generate a standard curve. The serum samples were prepared by 10-fold, 20-fold, and 40-fold dilutions in 5% BSA-PBST and added to the plate. The plate was incubated at room temperature for 1 hour and washed 5 times with PBST. 100 μl HRP-conjugated antibody (Bio-Rad, catalog no. HCA204P) was added to each well at 0.25 μg/mL in 5% BSA-PBST. The plate was incubated for 1 hour at room temperature and washed 5 times with PBST. 100 μl QuantaBlu (Thermo Scientific, catalog no. 15169) was added to each well. The fluorescence was measured after 10-15 minutes incubation at 325 nm/420 nm (emission/excitation). The titer of anti-TNFα in the serum samples was calculated against the standard curve.

The AUC₀₋₃₃₆ₕ for IgG-A was 150970, while IgG-A “F00” was 848924 ng/mL*hr (FIG. 10). The terminal half-life was estimated to be 33 hours and 152 hours, respectively. Thus, the single Phe mutation significantly improved the pharmacokinetic profile of the antibody in rat.

### Example 11

### Phe Mutation in Canine, Feline, and Equine IgG Fcs

The interaction between the Phe mutation in canine IgG-A, IgG-B, IgG-C, and IgG-D Fc and FcRn was modeled using three-dimensional protein structure analysis. The aromatic side chain of Phe appears to have a hydrophobic interaction with canine FcRn at the Pro hydrophobic ring (n-CH) of the “WPE” motif. In addition, the Phe hydrophobic side chain may be in direct contact with the Glu side chain next to the Pro of the same “WPE” motif. This interaction may have energy penalty if the Glu side chain is deprotonated to be negative charged, such as at a neutral pH. Thus, some level of protonation of the Glu residue may be required to minimize the aromatics to Glu-H interaction. That may explain why the interaction between variant IgGs having the Phe mutation and FcRn is reduced at neutral pH. Based on protein structure analysis, the interaction appears to be conserved among canine IgG-A, IgG-B, IgG-C, and IgG-D Fc.

Furthermore, the interactions between a Phe mutation in feline IgG1a and IgG2 Fc were modeled when complexed with feline FcRn. The same interactions observed with the canine IgG Fcs appeared to be conserved with the feline IgG Fcs.

The interactions between a Phe mutation in equine IgG1, IgG2, IgG3, IgG4, IgG5, IgG6, and IgG7 Fc in complex with equine FcRn were also modeled. The same interactions appeared to be maintained with the equine IgG Fcs.

### Example 12

### Other Exemplary Variant Canine IgG Fcs Enhance Canine FcRn Interaction

The affinity of additional variant canine Fc polypeptides for FcRn was evaluated in the context of a chimeric antibody. Antibody variable light chain fused to canine kappa light chain and variable heavy chain sequences fused to wild-type IgG-B Fc polypeptide (comprising SEQ ID NO: 39), variant canine IgG-B Fc polypeptide 0Y0 (comprising SEQ ID NO: 122), variant canine IgG-B Fc polypeptide 0YH (comprising SEQ ID NO: 123), variant canine IgG-B Fc polypeptide 0YY (comprising SEQ ID NO: 124), and variant canine IgG-B Fc polypeptide 00Y (comprising SEQ ID NO: 125) were expressed.

The binding analysis was performed using a biosensor OctetRed as follows. Briefly, biotinylated target was captured on streptavidin sensor tips. The association of antibody at 20 μg/mL was bound to the biotinylated target. The complex was then used to bind to canine FcRn (50 μg/mL) at pH 6.0. Dissociation was performed at pH 7.2.

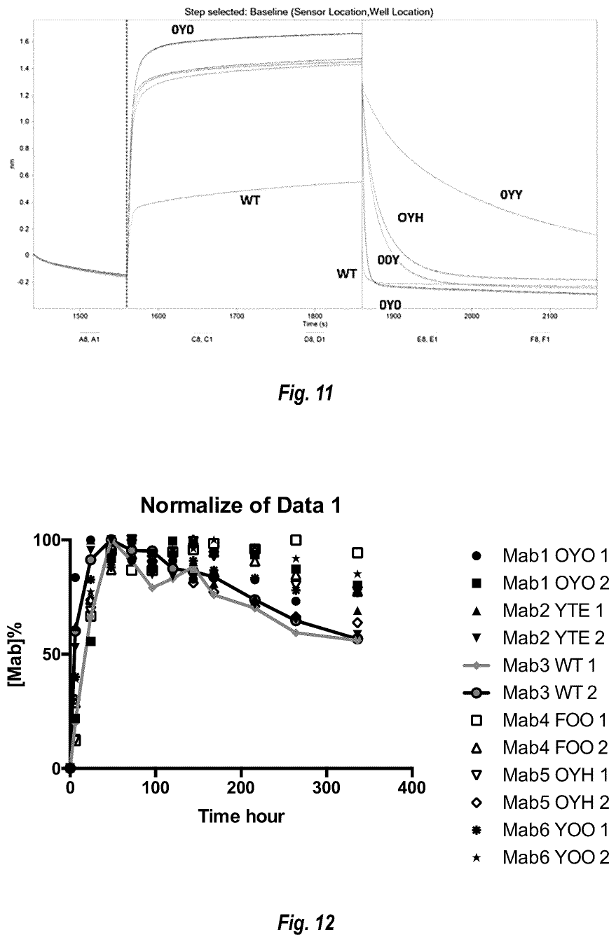

Each of the chimeric variant canine IgG-B antibodies exhibited enhanced binding to canine FcRn at pH 6.0 compared to the chimeric wild-type canine IgG-B antibody and each had an appreciable rate of dissociation at neutral pH (FIG. 11).

### Example 13

### Variant Canine IgG Fcs Extend Half-Life of Antibodies In Vivo in Canine

In vivo half-life of variant canine Fc polypeptides for FcRn was evaluated in the context of a chimeric antibody. Antibody variable light chain fused to canine kappa light chain and variable heavy chains fused to wild-type IgG-B Fc polypeptide (comprising SEQ ID NO: 39), variant canine IgG-B Fc polypeptide YTE (comprising SEQ ID NO: 126), variant canine IgG-B Fc polypeptide 0Y0 (comprising SEQ ID NO: 122), variant canine IgG-B Fc polypeptide F00 (comprising SEQ ID NO: 116), variant canine IgG-B Fc polypeptide 0YH (comprising SEQ ID NO: 123), and variant canine IgG-B Fc polypeptide Y00 (comprising SEQ ID NO: 117) were expressed and purified to 40 mg/mL in PBS, pH7.2.

Canine pharmacokinetics were performed at Absorption Systems California, LLC. Male beagles (˜8-14 kg) were obtained from Marshall Bioresources, North Rose, New York. A total of 12 dogs were used for study with n=2 dogs per group. The six antibodies were subcutaneously administered to the dogs at 4 mg/Kg. Serum samples were collected at pre-injection and at 6, 24, 48, 72, 96, 120, 144, 168, 216, 264, 336, 504 and 672 hours post-injection. The canine chimeric antibody concentrations were determined by ELISA as described. The Cp between time at 144 hour and 336 hour was transformed to Ln [Cp], then fit to linear equation in the form of Ln[Cp]ₜ=−k*t+Ln[Cp]₁₄₄ₕ. The terminal half-life was then calculated from slope k, as listed in Table 3, below. The 0Y0, F00, 0YH, and Y00 mutations in canine IgG-B Fc greatly improved the half-life of the antibody in vivo in dogs. The percent antibody normalized over time resulting from study is shown in FIG. 12.

TABLE 3
Effect of variant canine IgG Fcs on antibody half-life in dog

| Dog | Half-life (days) |
|---|---|
| WT 1 | 13 |
| WT 2 | 13 |
| YTE 1 | *21 |
| YTE 2 | 15 |
| 0Y0 1 | *65 |
| 0Y0 2 | 28 |
| F00 1 | *very long |
| F00 2 | 23 |
| 0YH 1 | 22 |
| 0YH 2 | 23 |
| Y00 1 | 33 |
| Y00 2 | 39 |

*data may not be reliable due to poor curve fitting

### Example 14

### Extracellular Domains of IL13 Receptor Decoy

Extracellular domains of mammalian IL13 receptor decoy (IL13Rd), such as IL13Rd of canine, feline and equine are capable of binding IL13. The IL13 binding domains of canine, feline, and equine IL13Rd were identified, and boundaries defined. Full-length precursor sequences for canine, feline, and equine IL13Rd correspond to SEQ ID NO: 164, SEQ ID NO: 165, and SEQ ID NO: 166, respectively. Exemplary extracellular domains of canine, feline, and equine IL13Rd were identified as SEQ ID NO: 167, SEQ ID NO: 168, and SEQ ID NO: 169, respectively.

Nucleotide sequences encoding canine IL13Rd ECD/IL4R ECD contiguous polypeptides linked to an IgGB Fc polypeptide with a signal sequence were designed, and the contiguous polypeptides were expressed and purified. Binding assays to canine IL13 were performed. All three contiguous polypeptides bound IL13 with kinetics requisite for therapeutic activity. In all cases, the Kd were in the nM range.

### Example 15

### IL13Rd ECD/IL4R ECD Contiguous Polypeptides and Heterodimeric Proteins

Contiguous IL13Rd ECD/IL4R ECD polypeptides may have the following formats:
Formula (I): IL13Rd-L1-IL4R-L2-FP,
Formula (II): IL4R-L1-IL13Rd-L2-FP,
Formula (III): IL13Rd-L1-FP-L2-IL4R,
Formulat (IV): IL4R-L1-FP-L2-IL13Rd,
Formula (V): FP-L1-IL13Rd-L2-IL4R, or
Formula (VI): FP-L1-IL4R-L2-IL13Rd,
   wherein IL13Rd is an IL13Rd extracellular domain (ECD) polypeptide from a companion animal species, IL4R is an IL4R ECD polypeptide from a companion animal species, L1 is a first optional linker, L2 is a second optional linker, and FP is an optional fusion partner, such as an Fc polypeptide.

In addition to contiguous IL13Rd/IL4R ECD polypeptide formats, heterodimeric protein pairs may have the following formats:
Heterodimeric protein A:
   Polypeptide 1: IL13Rd(n)-L-Fc1 and
   Polypeptide 2: IL4R(n)-L-Fc2; or
Heterodimeric protein B:
   Polypeptide 1: IL4R(n)-L-Fc1 and
   Polypeptide 2: IL13Rd(n)-L-Fc2,
      wherein IL13Rd(n) is at least one IL13Rd extracellular domain (ECD) polypeptide from a companion animal species, IL4R(n) is at least one IL4R ECD polypeptide from a companion animal species, (n) is one, two, three, four, or more ECD polypeptides, L is an optional linker, Fc1 is a variant Fc polypeptide, such as a variant Fc polypeptide comprising knob mutation, Fc2 is a variant Fc polypeptide, such as a variant Fc polypeptide comprising a hole mutation. An optional linker could also be used between multiple ECD polypeptides. In addition, other binding partner(s) may be included before, after, and/or between any one or more ECD polypeptide(s). Other potential binding partners include: IL5, IL6, IL17, IL22, IL31, LFA-1, TNF-α, TSLP, and/or IgE. A host cell may be cotransfected with vectors expressing these contiguous polypeptide pairs to produce the heterodimeric proteins described.

1-2. (canceled)

3. A contiguous polypeptide comprising an extracellular domain of an IL13R polypeptide and an extracellular domain of an IL4R polypeptide, wherein the IL13R and IL4R polypeptides are from a companion animal species, wherein the contiguous polypeptide comprises the formula (III) IL13R-L1-FP-L2-IL4R, formula (IV) IL4R-L1-FP-L2-IL13R, (V) FP-L1-IL13R-L2-IL4R, or formula (VI) FP-L1-IL4R-L2-IL13R, wherein:
a) IL13R is an extracellular domain of an IL13R polypeptide from the companion animal species,
b) IL4R is an extracellular domain of an IL4R polypeptide from the companion animal species,
c) L1 is a first optional linker,
d) L2 is a second optional linker, and
e) FP is a fusion partner, optionally an IgG Fc polypeptide.

4. The contiguous polypeptide of claim 3, wherein the contiguous polypeptide comprises a variant IgG Fc polypeptide from a companion animal species capable of binding to neonatal Fc receptor (FcRn) with an increased affinity relative to the wild-type Fc polypeptide, such as at a low pH.

5-10. (canceled)

11. The contiguous polypeptide of claim 3, wherein the extracellular domain of the IL13R polypeptide is at least 85% identical to the amino acid sequence of SEQ ID NO: 22, SEQ ID NO: 24, SEQ ID NO: 26, SEQ ID NO: 32, SEQ ID NO: 34, or SEQ ID NO: 36.

12-14. (canceled)

15. The contiguous polypeptide of claim 3, wherein the extracellular domain of the IL13R polypeptide comprises a cysteine at a position corresponding to position 18 of SEQ ID NO: 22, corresponding to position 18 of SEQ ID NO: 24, or corresponding to position 18 of SEQ ID NO: 26.

16. (canceled)

17. The contiguous polypeptide of claim 3, wherein the extracellular domain of the IL13R polypeptide comprises an amino acid sequence selected from SEQ ID NO: 32, SEQ ID NO: 34, and SEQ ID NO: 36.

18. The contiguous polypeptide of claim 3, wherein the extracellular domain of the IL13R polypeptide comprises an amino acid sequence selected from SEQ ID NO: 22, SEQ ID NO: 24, and SEQ ID NO: 26.

19. (canceled)

20. The contiguous polypeptide of claim 3, wherein the extracellular domain of the IL4R polypeptide is at least 85% identical to the amino acid sequence of SEQ ID NO: 23, SEQ ID NO: 163, SEQ ID NO: 25, SEQ ID NO: 27, SEQ ID NO: 33, SEQ ID NO: 35, or SEQ ID NO: 37.

21-23. (canceled)

24. The contiguous polypeptide of claim 3, wherein the extracellular domain of the IL4R polypeptide comprises an amino acid sequence selected from SEQ ID NO: 33, SEQ ID NO: 35, and SEQ ID NO: 37.

25. The contiguous polypeptide of claim 3, wherein the extracellular domain of the IL4R polypeptide comprises an amino acid sequence selected from SEQ ID NO: 23, SEQ ID NO: 163, SEQ ID NO: 25, and SEQ ID NO: 27.

26. The contiguous polypeptide of claim 3, wherein L1 and L2, if present, each independently comprises an amino acid sequence selected from G, GG, GGG, S, SS, SSS, GS, GSGS (SEQ ID NO: 151), GSGSGS (SEQ ID NO: 152), GGS, GGSGGS (SEQ ID NO: 153), GGSGGSGGS (SEQ ID NO: 154), GGGS (SEQ ID NO: 155), GGGSGGGS (SEQ ID NO: 156), GGGSGGGSGGGS (SEQ ID NO: 157), GSS, GSSGSS (SEQ ID NO: 158), GSSGSSGSS (SEQ ID NO: 159), GGSS (SEQ ID NO: 160), GGSSGGSS (SEQ ID NO: 161), and GGSSGGSSGGSS (SEQ ID NO: 162).

27. The contiguous polypeptide of claim 3, wherein the contiguous polypeptide comprises the sequence selected from SEQ ID NO: 13, SEQ ID NO: 14, SEQ ID NO: 15, SEQ ID NO: 16, SEQ ID NO: 17, SEQ ID NO 18, SEQ ID NO: 19, SEQ ID NO: 20, SEQ ID NO: 21, SEQ ID NO: 28, SEQ ID NO: 29, SEQ ID NO: 30, and SEQ ID NO: 31.

28. A heterodimeric protein comprising:
a) a first contiguous polypeptide comprising at least one IL13R decoy extracellular domain (ECD) and a first Fc polypeptide, and
b) a second contiguous polypeptide comprising at least one IL4R ECD and a second Fc polypeptide,
wherein the IL13R decoy ECD and/or the IL4R ECD are from a companion animal species.

29. The heterodimeric protein of claim 28, wherein the first Fc polypeptide and/or the second Fc polypeptide is a variant IgG Fc polypeptide from a companion animal species capable of binding to neonatal Fc receptor (FcRn) with an increased affinity relative to the wild-type Fc polypeptide, optionally at a low pH.

30-64. (canceled)

65. An isolated polypeptide comprising the amino acid sequence of SEQ ID NO: SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, or SEQ ID NO: 183.

66. (canceled)

67. An isolated nucleic acid encoding the contiguous polypeptide of claim 3.

68. A host cell comprising the nucleic acid of claim 67.

69. (canceled)

70. A method comprising culturing the host cell of claim 68 and isolating the contiguous polypeptide.

71. (canceled)

72. A method of treating a companion animal species having an IL13 and/or 1L4-induced condition, the method comprising administering to the companion animal species a therapeutically effective amount of the contiguous polypeptide of claim 3.

73-78. (canceled)

79. A method of reducing IL13 and/or 1L4 signaling activity in a cell, the method comprising exposing the cell to the contiguous polypeptide of claim 3 under conditions permissive for binding of the heterodimeric protein to 1L13 and/or 1L4, thereby (a) reducing binding of IL/4 and/or IL-13 to native IL13 receptor and/or native IL-4 receptor and (b) reducing IL13- and/or IL-4-mediated signaling.

80-82. (canceled)

83. A method for detecting IL13 or IL4 in a sample from a companion animal species comprising contacting the sample with the contiguous polypeptide of claim 3 under conditions permissive for binding of the heterodimeric protein to IL13 and/or I14, and detecting whether a complex is formed between the heterodimeric protein and IL13 and/or I14 in the sample.

84. (canceled)
